(19) United States
(12) Patent Application Publication
(10) Pub. No.: US 2023/0060409 A1
(43) Pub. Date: Mar. 2, 2023

(54) Methods of Treatment of Neurofibromatosis Type 1 (NF1) and NF-1 Mediated Conditions and Compositions for Use in Such Methods

(71) Applicants: The UAB Research Foundation, Birmingham, AL (US); Royal Holloway and Bedford New College,, Surrey (GB)

(72) Inventors: Deeann WALLIS, Birmingham, AL (US); Robert KESTERSON, Birmingham, AL (US); Bruce KORF, Birmingham, AL (US); Andre LEIER, Birmingham, AL (US); Laura LAMBERT, Birmingham, AL (US); Linda POPPLEWELL, Surrey (GB); George DICKSON, Surrey (GB)

(21) Appl. No.: 17/762,355

(22) PCT Filed: Sep. 21, 2020
(86) PCT No.: PCT/US2020/051827

§ 371 Date: Mar. 21, 2022

Related U.S. Application Data
(60) Provisional application No. 62/903,521, filed on Sep. 20, 2019.

(51) Int. Cl. C12N 15/113

(52) U.S. Cl. CPC C12N 15/113; C12N 2310/11; C12N 2310/3233; C12N 2310/314; C12N 2320/33

(57) ABSTRACT

The present disclosure provides methods and compositions for the treatment of NF-1 and NF-1 mediated conditions. The present disclosure further provides for methods of exon skipping and exon retention and compositions for use in such methods. Such methods of exon skipping and exon retention may be used in the methods of treatment discussed herein. The present disclosure further provides new therapeutic compounds, particularly oligonucleotides, including antisense oligonucleotides, for use in the methods described herein.

### BACKGROUND

Neurofibromatosis type 1 (NF1) is a condition characterized by changes in skin coloring (pigmentation) and the growth of tumors along nerves in the skin, brain, and other parts of the body. The signs and symptoms of this condition vary widely among affected people.

NF1 is the most common single gene disorder in humans, occurring in about 1 in 2500-3000 births worldwide and has high phenotypic variability, with members of the same family with the same mutation displaying different symptoms and symptom intensities.

Most adults with NF1 develop neurofibromas, which are noncancerous (benign) tumors that are usually located on or just under the skin. These tumors may also occur in nerves near the spinal cord or along nerves elsewhere in the body. Some people with NF1 develop cancerous tumors that grow along nerves. These tumors, which usually develop in adolescence or adulthood, are called malignant peripheral nerve sheath tumors (MPNSTs). People with NF1 also have an increased risk of developing other cancers, including, but not limited to, brain tumors, breast cancer, melanoma, and cancer of blood-forming tissue (including, but not limited to, leukemia, including juvenile myelomonocytic leukemia) and other conditions, including, but not limited to, Watson syndrome, Lisch nodules, vision abnormalities, behavioral disorders, attention deficit hyperactivity disorder (ADHD), cognitive disorders, high blood pressure (hypertension), short stature, an unusually large head (macrocephaly), and skeletal abnormalities (such as, but not limited to, an abnormal curvature of the spine; scoliosis).

During childhood, benign growths called Lisch nodules often appear in the colored part of the eye (the iris). Lisch nodules do not interfere with vision. Some affected individuals also develop tumors that grow along the nerve leading from the eye to the brain (the optic nerve). These tumors, which are called optic gliomas, may lead to reduced vision or total vision loss. In some cases, optic gliomas have no effect on vision.

The NF1 gene provides instructions for making a protein called neurofibromin. This protein is produced in many cells, including nerve cells and specialized cells surrounding nerves (oligodendrocytes and Schwann cells). Neurofibromin acts as a tumor suppressor and functions to inhibit the Ras polypeptide and the Ras signaling pathway. Members of the Ras superfamily of signaling proteins modulate fundamental cellular processes by cycling between an active GTP-bound conformation and an inactive GDP-bound form. Mutations in the NF1 gene lead to the production of mutated/altered neurofibromin, many of which lack the ability to inhibit Ras signaling. When the Ras pathways is left unchecked, the result in cellular over-proliferation and tumor formation.

Neurofibromin has several predicted functional domains, with the best characterized and most significant being the GAP-related domain (GRD). The GRD functions by binding GTP-bound Ras and stimulating intrinsic GTPase activity in wild type Ras to return Ras to its inactive GDP-bound state. The cysteine-serine rich domain (CSRD), SEC-PH domain, and C-terminal domain are little characterized, and their requirement for proper neurofibromin function is presumed due to pathogenic variants in these regions. Through the GRD domain, neurofibromin increases the rate of GTP hydrolysis of Ras, and acts as a tumor suppressor by reducing Ras activity.

NF1 is considered to have an autosomal dominant pattern of inheritance. People with this condition are born with one mutated copy of the NF1 gene in each cell. In about half of cases, the altered gene is inherited from an affected parent. The remaining cases result from de novo mutations in the NF1 gene and occur in people with no history of the disorder in their family. Unlike most other autosomal dominant conditions, in which one altered copy of a gene in each cell is sufficient to cause the disorder, two copies of the NF1 gene must be altered to trigger tumor formation in NF1. In many cases, a mutation in the second copy of the NF1 gene occurs during a person's lifetime in specialized cells surrounding nerves. Almost everyone who is born with one NF1 mutation acquires a second mutation in one or more cell types and develops the tumors characteristic of NF1. Mutations in NF1 are primarily associated with NF1 (also known as von Recklinghausen syndrome) although are present in other conditions (for example, certain types of cancers).

NF1 is a widespread and serious disease impacting the lives of countless individuals. Furthermore, due to the large number of pathogenic variants giving rise to NF1, it has been difficult to develop therapeutic approaches applicable to the general treatment of NF1. While there are current treatments available for NF1 and other conditions involving mutations in the NF1 gene, such treatments are either not widely effective and/or are accompanied by severe side effects. Most currently available drugs being tested to treat NF1 are targeted at tumors and have focused on blocking Ras signaling or interfering with intercellular communication. For example, MEK inhibitors (such as selumetinib) have been partially successful in the treatment of plexiform neurofibromas, however, not all patients benefit and the plexiform neurofibromas do not completely disappear. Furthermore, the administration of MEK inhibitors can result in significant side effects. Additional treatments that can be used alone or in conjunction with current treatments, for example inhibitor of Ras signaling, including MEK inhibitors, are therefore needed.

In addition, the NF1 gene may also serve as a driver of carcinogenesis in individuals (including those lacking a germline mutation of the NF1 gene). Mutations in the NF1 gene may drive initial tumor formation or may occur subsequent to tumor formation and further stimulate tumor formation and/or progression.

Therefore, the art is in need of new treatments for NF1-mediated conditions and methods to determine appropriate treatments for individuals with an NF1-mediated condition. The present disclosure provides a solution to both of these problems in the art.

### BRIEF DESCRIPTION OF THE FIGURES

FIG. 1 shows in silico analysis of NF1. Domain indicates those exons that contribute to the formation of a known (or suspected) functional protein domain. The C-Terminal Domain includes the nuclear localization signal and the binding region for Syndecans. Also, Ser 2808 is a known phosphorylation site in exon 57 that is involved in nuclear localization. Exon # indicates exon number. Exons (E) that underwent in-depth in silico analysis and that were tested in vitro are marked in gray with dark border. In-Frame? Indicates single exons that, when skipped, produce an in-frame deletion (without producing a missense mutation) are marked in green. Single exons that, when skipped, produce a frameshift, leading to a truncated protein, are marked in red. The same holds for consecutive exon pairs that when skipped individually lead to a frameshift but when skipped together result in an in-frame deletion. Skipping of exon 1 (marked gray) leads to an in-frame deletion. The new start codon is at the end of exon 2, resulting in a 2751 amino acid protein. Skipping of exons 55 and 56 (marked gray) is an in-frame deletion with an additional missense mutation. In addition, in principle, exons 56 and 57 can also be skipped, but this would include skipping the stop codon at the end of the gene. Length indicates the number of nucleotides contributed by each exon. The longer the exon, the higher the probability that it provides crucial functionality to the protein and the darker the color code. Patient: The LOVD 3.0 (Build 21) and literature were searched for reports of genomic variants with individual exons deleted. An exon is in dark red if a patient with NF1 has been reported that has that exon deleted in transcripts due to a mutation. PTM indicates exons with known, experimentally verified post-translational modifications (PTMs), in particular phosphorylation, ubiquitination, and acetylation (in human and/or murine tissue). Phosphorylation is viewed as likely more important for NF1 function than other PTMs. Consequently, exons containing residues that have been experimentally verified to be phosphorylated are marked in dark red, while all others are marked in pink. Numbers refer to number of modified residues in a given exon. Disorder indicates the number of disordered residues. Amino acids are counted towards the exon that provides at least two nucleotides. Prediction obtained from MD (MetaDisorder). MD results are provided as part of the ProteinPredict output. MD includes four predictors, namely PROFbval, DISOPRED2, Ucon and NORSnt. The output is summarized (MD2st; the two-state prediction by MD) for each exon. NORS indicates the percentage of Non-ORdinary Secondary structure, i.e. unstructured loops, contributed by each exon. This is obtained using NORSnet. Average Conservation: Average conservation score for each exon as calculated by ConSurf. Maximum Conservation indicates the number of amino acids with the highest conservation score for each exon as obtained from ConSurf.

FIG. 2 shows a summary of in silico analysis for selected exons of NF1. Exon(s) skipped indicates the number of exon(s) skipped according to continuous 1-58 exon numbering (human neurofibromin, 2818 aa-long isoform). Predicted secondary structure (%) indicates the percentage of residues in the remaining protein (NF1delEX) that are predicted to undergo a change in secondary structure when compared to (full length) human neurofibromin. Highest is reliability (secondary structure) indicates predictions of secondary structures have a reliability score assigned. Here the highest reliability reported for any such prediction are provided. Predicted solvent accessibility (%) indicates the percentage of residues in the remaining protein (NF1delEX) that are predicted to undergo a change in solvent accessibility when compared to predictions for (full length) human neurofibromin. Highest reliability (solvent accessibility) indicates predictions of solvent accessibility with a reliability score assigned. Here the highest reliability reported for any prediction are reported. Surface contributed by exon (Å²) indicates predicted solvent accessibility in squared Angstrom attributed to the amino acids that have been translated from the exon(s). Change of surface area (Å²) indicates predicted total solvent accessibility in squared Angstrom of human neurofibromin minus the predicted surface area contributed by the skipped exon(s) minus the predicted solvent accessibility of the protein with skipped exon. Predicted O->D (#) indicates the number of residues in the shortened protein (NF1delEX) predicted to change status from ordered to disordered, when compared to full length neurofibromin. Predicted D->O (#) indicates the number of residues in the shortened protein (NF1delEX) predicted to change status from disordered to ordered, when compared to full length neurofibromin. Highest reliability (Ordered/Disordered) indicates predictions of status (ordered versus disordered) and have a reliability score assigned. Here the highest reliability reported for any prediction is provided. Predicted P-P binding sites (#) indicates the number of predicted Protein-Protein binding sites as predicted by PROFisis (part of ProteinPredict). Predicted PTMs indicate the number of PTMs as predicted from Prosite as part of ProteinPredict, CKSAAP_UbSite and UbiProber (with score >0.8). This includes PTMs that are not on residues formed by the exon but likely affected by the exon skipping, if the recognition sequence is adjacent to the skipped region.

FIG. 3A shows a representative Western blot of NF1 and tubulin levels of mNF1 cDNAs with selected exon skips.

FIG. 3B shows quantitation of NF1/tubulin ratios normalized to WT ratio of mNF1 cDNAs with selected exon skips. N>3; error bars represent SEM.

FIG. 4A shows GTP-RAS levels normalized to WT and compared to EV control of mNF1 cDNAs with selected exon skips. N>3; error bars represent SEM; +−p<0.01; *−p<0.05; **−p<0.01.

FIG. 4B shows representative Western blot of p-ERK/ERK ratios of mNF1 cDNAs with selected exon skips.

FIG. 4C shows quantitation of p-ERK/ERK ratios normalized to WT and compared to EV control of mNF1 cDNAs with selected exon skips. N<3; error bars represent SEM; *−p<0.05; **−p<0.01.

FIG. 4D shows ELK1 transcriptional activity normalized to WT and compared to EV control of mNF1 cDNAs with selected exon skips N<3; error bars represent SEM*−p<0.05; **−p<0.01.

FIG. 5A is an overview of the creation of Nf1 exon 17 deletion mice (DelE17), showing a schematic view of murine Nf1 genomic region with intron and exon boundaries. The top sequence depicts the sense strand of the wild-type allele with canonical splice sites in bold text and the exon sequence underlined. Arrow heads represent the beginning and end of the deleted region. Black bars depict Cas9 Guide sequences both 5′ and 3′ of exon 17. The bottom sequence depicts the sense strand of the mutant allele.

FIG. 5B shows the results of RT-PCR analysis of exon 17 in a wild-type and DelE17 mouse illustrating the transcript from the DelE17 mouse is shorter than the transcript from the wild-type mouse confirming deletion of exon 17.

FIG. 5C shows a comparison of an Nf1 exon 17 deletion mouse as compared to an NF1 wild-type littermate.

FIG. 6A shows analysis of the NF1 exon 17 and 100 bp of upstream and downstream flanking introns using Human Splice Finder. Antisense oligonucleotides (ASO) were designed to target the positive peaks (indicating presence of ESE motifs, pink and red bars) and avoid negative troughs (indicating presence of ESS motifs, green and blue bars).

FIG. 6B shows secondary structure of the NF1 exon 17 and 100 bp of upstream and downstream flanking introns modelled using Visual OMP software.

FIG. 6C shows a summary table outlining ASO sequences designed and their target sequences and includes percentage GC content, ΔG value in kcal mol⁻¹ (overall binding energy), number of target open conformations spanned by each ASO and percentage of ASO nucleotides binding within open conformation of the target.

FIG. 7A shows analysis of the NF1 exon 46 and 100 bp of upstream and downstream flanking introns using Human Splice Finder to identify ASO for NF1 exon 46 skipping. ASOs were designed to target the positive peaks (indicating presence of ESE motifs, pink and red bars) and avoid negative troughs (indicating presence of ESS motifs, green and blue bars).

FIG. 7B shows secondary structure of the NF1 exon 46 and 100 bp of upstream and downstream flanking introns modelled using Visual OMP software.

FIG. 7C shows a summary table outlining ASO sequences designed and their target sequences and includes percentage GC content, ΔG value in kcal mol⁻¹ (overall binding energy), number of target open conformations spanned by each ASO and percentage of ASO nucleotides binding within open conformation of the target.

FIG. 8A shows analysis of the NF1 exon 51 and 100 bp of upstream and downstream flanking introns using Human Splice Finder to identify ASO for NF1 exon 51 skipping. ASOs were designed to target the positive peaks (indicating presence of ESE motifs, pink and red bars) and avoid negative troughs (indicating presence of ESS motifs, green and blue bars).

FIG. 8B shows secondary structure of the NF1 exon 51 and 100 bp of upstream and downstream flanking introns modelled using Visual OMP software.

FIG. 8C shows a summary table outlining ASO sequences designed and their target sequences and includes percentage GC content, ΔG value in kcal mol⁻¹ (overall binding energy), number of target open conformations spanned by each ASO and percentage of ASO nucleotides binding within open conformation of the target.

FIG. 9A shows analysis of the NF1 exon 13 and 100 bp of upstream and downstream flanking introns using Human Splice Finder to identify ASO for NF1 exon 13 retention. ASOs were designed to avoid the positive peaks (indicating presence of ESE motifs, pink and red bars) and target the cryptic splice site mutation (black arrow).

FIG. 9B shows secondary structure of the NF1 exon 13 and 100 bp of upstream and downstream flanking introns modelled using Visual OMP software.

FIG. 9C shows a summary table outlining ASO sequences designed and their target sequences and includes percentage GC content, ΔG value in kcal mol⁻¹ (overall binding energy), number of target open conformations spanned by each ASO and percentage of ASO nucleotides binding within open conformation of the target.

FIG. 10A shows screening of designed 25-mer ASOs (hNF1.e17[+79+103], hNF1.e17[+82+106], hNF1.e17[+85+109], hNF1.e17[+89+112], hNF1.e17[+92+115], and hNF1.e17[+95+118]) for exon skipping efficiency of exon 17 in HEK293 cells expressing wild-type NF using a nested PCR readout.

FIG. 10B shows the effect of increasing dose (concentration of 1 μM to 20 μM) of 25-mer ASO hNF1.e17[+79+103] on exon skipping efficiency in HEK293 cells.

FIG. 10C shows quantification of the data of FIG. 10B using ImageJ Software, indicating the percentage of exon 17 skipping at each concentration.

FIG. 10D shows the effect of increasing dose (concentration of 1 μM to 20 μM) of 25-mer ASO hNF1.e17[+85+109] on exon skipping efficiency in HEK293 cells.

FIG. 10E shows quantification of the data of FIG. 10D using ImageJ Software, indicating the percentage of exon 17 skipping at each concentration.

FIG. 11A shows the effect of increasing dose (concentration of 500 nM to 10 μM) of 28-mer ASO hNF1.e17[+79+106] on exon 17 skipping efficiency in HEK293 cells.

FIG. 11B shows the effect of increasing dose (concentration of 10 nM to 10 μM) of 28-mer ASO hNF1.e47[+76+103] on exon 47 skipping efficiency in HEK293 cells.

FIG. 11C shows the effect of increasing dose (concentration of 10 nM to 4 μM) of 28-mer ASO hNF1.e52[+51+78] on exon 52 skipping efficiency in HEK293 cells.

FIG. 11D shows the effect of increasing dose (concentration of 5 nM to 4 μM) of 28-mer ASO hNF1.e52[+50+77] on exon 52 skipping efficiency in HEK293 cells.

FIG. 12A shows NF1/actin ratios normalized to WT in HEK293 cells expressing wild-type NF1 or NF1 containing the c.1885G>A mutation, demonstrating the c.1885G>A mutation completely inhibits production of neurofibromin. Error bars represent SEM; n=3.

FIG. 12B shows GTP-Ras levels normalized to WT in HEK293 cells expressing wild-type NF1 or NF1 containing the c.1885G>A mutation, demonstrating increased GTP-Ras levels in the absence of neurofibromin polypeptide. Error bars represent SEM; n=3.

FIG. 12C shows pERK/ERK ratios normalized to WT in HEK293 cells expressing wild-type NF1 or NF1 containing the c.1885G>A mutation, demonstrating increased pERK levels in the absence of neurofibromin polypeptide. Error bars represent SEM; n=3.

FIG. 13A shows NF1/actin ratios normalized to WT in HEK293 cells expressing wild-type NF1 or NF1 containing the c.6948insT mutation, demonstrating the c.6948insT mutation significantly inhibits production of neurofibromin. Error bars represent SEM; n=3.

FIG. 13B shows GTP-Ras levels normalized to WT in HEK293 cells expressing wild-type NF1 or NF1 containing the c.6948insT mutation, demonstrating increased GTP-Ras levels when neurofibromin polypeptide levels are decreased. Error bars represent SEM; n=3.

FIG. 13C shows pERK/ERK ratios normalized to WT in HEK293 cells expressing wild-type NF1 or NF1 containing the c.6948insT mutation, demonstrating increased pERK levels when neurofibromin polypeptide levels are decreased. Error bars represent SEM; n=3.

FIG. 14A shows NF1/actin ratios normalized to WT in HEK293 cells expressing wild-type NF1 or NF1 containing the c.7648A>T mutation, demonstrating the c.7648A>T mutation significantly inhibits production of neurofibromin. Error bars represent SEM; n=3.

FIG. 14B shows GTP-Ras levels normalized to WT in HEK293 cells expressing wild-type NF1 or NF1 containing the c.7648A>T, demonstrating increased GTP-Ras levels when neurofibromin polypeptide levels are decreased. Error bars represent SEM; n=3.

FIG. 14C shows pERK/ERK ratios normalized to WT in HEK293 cells expressing wild-type NF1 or NF1 containing the c.7648A>T mutation, demonstrating increased pERK levels when neurofibromin polypeptide levels are decreased. Error bars represent SEM; n=3.

FIG. 15A shows NF1/actin ratios in HEK293 cells expressing NF1 containing c.1885G>A mutation in exon 17 without added ASO (con; black bars) and with 10 uM hNF1.e17[+79+106] ASO (SEQ ID NO: 49; white bars) and NF1 containing c.7648A>T mutation in exon 52 without added ASO (con; black bars) and with 10 uM hNF1.e51[+51+78] ASO (SEQ ID NO: 53; white bars). The results show the added ASOs are capable of inducing exon skipping of exons 17 and 52 and partially restore neurofibromin expression; n=1.

FIG. 15B shows pERK/ERK ratios in HEK293 cells expressing NF1 containing c.1885G>A mutation in exon 17 without added ASO (con; black bars) and with 10 uM hNF1.e17[+79+106] ASO (SEQ ID NO: 49; white bars) and NF1 containing c.7648A>T mutation in exon 52 without added ASO (con; black bars) and with 10 uM hNF1.e51[+51+78] ASO (SEQ ID NO: 53; white bars). The results show that exon skipping of exons 17 and 52 deceases the pERK/ERK ratio, indicating inhibition of Ras activity; n=1.

### DETAILED DESCRIPTION

The present disclosure provides methods and compositions for the treatment of NF-1 and NF-1 mediated conditions. The present disclosure further provides for methods of exon skipping and exon retention and compositions for use in such methods. Such methods of exon skipping and exon retention may be used in the methods of treatment discussed herein. The present disclosure further provides new therapeutic compounds, particularly oligonucleotides, including antisense oligonucleotides, for use in the methods described herein. Both in vitro and in vivo methods are provided for testing and evaluating the compounds disclosed as well as predicting and evaluating the effects of exon skipping and exon retention on NF-1 gene activity.

### Definitions

As used herein, an NF1-mediated condition is a disease or condition that is caused, at least in part, by a decrease in neurofibromin function. Such a decrease in function may be caused by or be the result of a mutation in the NF1 gene that results in a neurofibromin polypeptide that does not function properly (as compared to a WT neurofibromin polypeptide). In one embodiment, a mutation in the NF1 gene results in a neurofibromin polypeptide having decreased protein levels, decreased stability, decreased trafficking and/or incorrect cellular localization, decreased activity, or a combination of any of the foregoing. In another embodiment, a mutation in the NF1 gene results in a neurofibromin polypeptide having decreased protein levels, decreased stability, decreased activity, or a combination of the foregoing. In another embodiment, a mutation in the NF1 gene results in a neurofibromin polypeptide having decreased protein levels, decreased activity, or a combination of the foregoing. Representative NF-1 mediated conditions include, but are not limited to, NF1, neurofibromas (including, but not limited to, malignant peripheral nerve sheath tumors, diffuse neurofibromas, cutaneous neurofibromas, intramuscular neurofibromas, plexiform neurofibromas, solitary neurofibroma, Schwannomas, and nerve root neurofibroma), cancer (including, but not limited to, brain tumors, cancer of blood-forming tissue juvenile myelomonocytic leukemia and other leukemia, optic glioma, breast cancer, and melanoma), Lisch nodules, Watson syndrome, high blood pressure (hypertension), short stature, an unusually large head (macrocephaly), and skeletal abnormalities (such as, but not limited to, an abnormal curvature of the spine; scoliosis), learning disabilities, ADHD, behavioral disorders, cognitive impairment, epilepsy, sphenoid bone dysplasia, and congenital hydrocephalus and associated neurologic impairment. In a particular embodiment, the NF1-mediated condition is NF1. A NF1-mediated condition may include a condition in which the subject does not have a germline mutation in the NF1 gene.

As used herein, the term “about” refers to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the terms “animal,” “subject” and “patient” as used herein include all members of the animal kingdom including, but not limited to, mammals, animals (e.g., cats, dogs, horses, swine, etc.) and humans. In certain embodiments, the subject is a human.

As used herein, the terms “antisense oligonucleotide,” “antisense oligomer” or “antisense compound” are used interchangeably and refer to a sequence of subunits, each bearing a base-pairing moiety, linked by intersubunit linkages that allow the base-pairing moieties to hybridize to a target sequence in a nucleic acid (typically an RNA, such as a pre-MRNA) by Watson-Crick base pairing, to form a nucleic acid:antisense oligomer heteroduplex within the target sequence. The subunits may be based on ribose or another pentose sugar or, in a preferred embodiment, a morpholino group (see description of morpholino oligomers below).

An antisense oligomer can be designed to block or inhibit translation of mRNA or to inhibit natural pre-mRNA splice processing, and may be said “to target” or to be “targeted against” a target sequence with which it hybridizes. In certain embodiments, the target sequence includes a region including a 3′ or 5′ splice site of a pre-mRNA, a exonic splicing enhancer site, or a spice site formed by mutation (such as a cryptic splice site). The target sequence may be within an exon or within an intron. In certain embodiments, the target sequence for a splice site may include an mRNA sequence having at its 5′ end 1 to about 35 base pairs downstream of a normal splice acceptor junction in a preprocessed mRNA. In certain embodiments, the target sequence for a splice site may include an mRNA sequence having at its 5′ end 1 to about 35 base pairs downstream of an exonic splicing enhancer site in a preprocessed mRNA. In certain embodiments, the target sequence for a splice site may include an mRNA sequence having at its 5′ end 1 to about 25 base pairs downstream of a splice site created by mutation. Included are antisense oligomers that comprise, consist essentially of, or consist of one or more of the sequences of SEQ ID NOS: 1-24, 49, 51, or 53. Also included are variants of these antisense oligomers, including variant oligomers having 80%, 85%, 90%, 95%, 97%, 98%, or 99% (including all integers in between) sequence identity or sequence homology to any one of sequences of SEQ ID NOS: 1-24, 49, 51, or 53 and/or variants that differ from these sequences by about 1, 2, 3, 4, 5, 6, 7, 8, 9, or 10 nucleotides. Preferably such variants induce exon skipping or induce exon retention of one or more selected human NF-1 exons. Also included are antisense oligomers of any one of sequences of SEQ ID NOS: 1-24, 49, 51, or 53, or variants thereof, which comprise a suitable number of charged linkages, provided that the charged linkages do not exceed half the total number of linkages present, for example, up to about 1 charged linkage per every 2-5 uncharged linkages, such as about 4-5 charged linkages per every 10 uncharged linkages.

As used herein, the term an “effective amount” or “therapeutically effective amount” refers to an amount of therapeutic compound, such as an antisense oligomer, administered to a subject, which is effective to produce a desired physiological response and/or therapeutic effect in the subject. The actual dose which comprises the effective amount may depend upon the route of administration, the size and health of the subject, the disorder being treated, and the like. One example of a desired physiological response includes increased expression of a functional or biologically active form of neurofibromin polypeptide, as compared to the physiological response in the absence of an antisense oligomer. Another example of a desired physiological response includes stimulation of the GTPase activity of a Ras polypeptide (which can be measured directly at the level of the Ras polypeptide or at a downstream target that undergoes a modification of increased activity in response to Ras activation). An increased expression of a functional or biologically active form of neurofibromin polypeptide or stimulation of the GTPase activity of a Ras polypeptide is preferably determined by the methods described herein. Examples of desired therapeutic effects include, without limitation, improvements in the symptoms or pathology of NF-1 or an NF-1 mediated condition, reducing the progression of symptoms or pathology of NF-1 or an NF-1 mediated condition, and slowing the onset of symptoms or pathology of NF-1 or an NF-1 mediated condition, among others.

In some embodiments, the “effective amount” or “therapeutically effective amount” in the context of the present disclosure increases the expression of a functional or biologically active form of neurofibromin polypeptide in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “effective amount” or “therapeutically effective amount” in the context of the present disclosure increases the GTPase activity of a Ras polypeptide by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In some embodiments, the “effective amount” or “therapeutically effective amount” in the context of the present disclosure improves the symptoms or pathology of NF-1 or an NF-1 mediated condition, reduces the progression of symptoms or pathology of NF-1 or an NF-1 mediated condition, and/or slows the onset of symptoms or pathology of NF-1 or an NF-1 mediated condition, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In each of the foregoing, when a reduction of increase is specified, such reduction or increase may be determined with respect to a subject that has not been treated with a compound disclosed herein and that has a diagnosis of NF1.

As used herein, the term “exon” refers to a defined section of nucleic acid that encodes for a protein, or a nucleic acid sequence that is represented in the mature form of an RNA molecule after portions of a precursor RNA (for example, pre-mRNA) have been removed by splicing. The mature RNA molecule can be a messenger RNA (mRNA) or a functional form of a non-coding RNA, such as rRNA or tRNA. The human NF-1 gene has 58 exons.

As used herein, the term “exon retention” refers generally to the process by which an entire exon, or a portion thereof, is retained in a given precursor RNA (such as pre-mRNA), and is thereby included in the mature RNA (such as mRNA). For example, the portion of the protein that is encoded by the retained exon is present in the expressed form of the protein creating a functional form of the protein. In certain embodiments, the exon being retained is an exon from the human NF-1 gene that contains a mutation introducing a splice site into the exon, such as a cryptic splice site, which causes aberrant splicing when present. In certain embodiments, the exon being skipped is any one or more of exons 1-58 of the human NF-1 gene. In certain embodiments, the exon being skipped is exon 13 of the human NF-1 gene.

As used herein, the term “exon skipping” refers generally to the process by which an entire exon, or a portion thereof, is removed from a given precursor RNA (such as pre-mRNA), and is thereby excluded from being present in the mature RNA, such as mRNA. For example, the portion of the protein that is otherwise encoded by the skipped exon is not present in the expressed form of the protein, typically creating an altered, though in certain cases still functional, form of the protein. In certain embodiments, the exon being skipped is an aberrant exon from the human NF-1 gene, which may contain a mutation or other alteration in its sequence that otherwise causes abnormal splicing. In certain embodiments, the exon being skipped is any one or more of exons 1-58 of the human NF-1 gene. In certain embodiments, the exon being skipped is any one or more of exons 3, 7/8, 9, 10, 11, 12, 14, 17, 18/19, 20, 21, 24, 25, 36, 41, 46, 47, 49, and 52, preferably exons 9, 12, 17, 20, 21, 25, 36, 41, 47, and 52, more preferably exons 9, 12, 17, 25, 41, 47, and 52, and more preferably, exons 17, 47, and 52 of the NF1 gene.

As used herein, the term “intron” refers to a nucleic acid region (within a gene) that is not translated into a protein. An intron is a non-coding section that is transcribed into a precursor mRNA (for example, pre-mRNA) and subsequently removed by splicing during formation of the mature RNA (such as mRNA).

As used herein, the terms “morpholino oligomer” or “PMO” (phosphorodiamidate morpholino oligomer) refer to an oligonucleotide analog composed of morpholino subunit structures, where (i) the structures are linked together by phosphorus-containing linkages, one to three atoms long and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide, such as included in a target sequence. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, and PCT Appn. No. PCT/US07/11435 (cationic linkages), all of which are incorporated herein by reference.

The purine or pyrimidine base pairing moiety is typically adenine, cytosine, guanine, uracil, thymine or inosine. Also included are bases such as pyridin-4-one, pyridin-2-one, phenyl, pseudouracil, 2,4,6-trime115thoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, .beta.-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, R-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine (A), guanine (G), cytosine (C), thymine (T), and uracil (U), as illustrated above; such bases can be used at any position in the antisense oligonucleotide. The skilled person in the art will appreciate that depending on the uses of the oligomers, Ts and Us are interchangeable.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable salt” refers to physiologically and pharmaceutically acceptable salts of the compounds of the invention: i.e., salts that retain the desired biological activity of the parent compound and do not impart undesired toxicological effects to the compounds disclosed. For oligonucleotides, preferred examples of pharmaceutically acceptable salts include but are not limited to (a) salts formed with cations such as sodium, potassium, ammonium, magnesium, calcium, polyamines such as spermine and spermidine and the like; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “specifically hybridisable” refers to a sufficient degree of complementarity or precise pairing such that stable and specific binding occurs between the oligonucleotide and the nucleic acid target. It is understood in the art that the sequence of an oligonucleotide, such as an ASO, need not be 100% complementary to that of its target sequence to be specifically hybridisable. An oligonucleotide, such as an ASO, is specifically hybridisable when binding of the compound to the target nucleic acid interferes with the normal function of the target nucleic acid to cause a loss of utility or loss of function (such as, but not limited to the loss of function of a exon splicing enhancer or a cryptic splice site), and there is a sufficient degree of complementarity to avoid non-specific binding of the oligonucleotide, such as an ASO, to non-target sequences under conditions in which specific binding is desired, such as under physiological conditions in the case of in vivo assays or therapeutic treatment, and in the case of in vitro assays, under the conditions in which the assays are performed.

As used herein, the term “treatment,” “treating,” or “treat” refers to improving a symptom of a disease or disorder and may comprise curing the disorder, substantially preventing the onset of the disorder, or improving the subject's condition.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

### Background

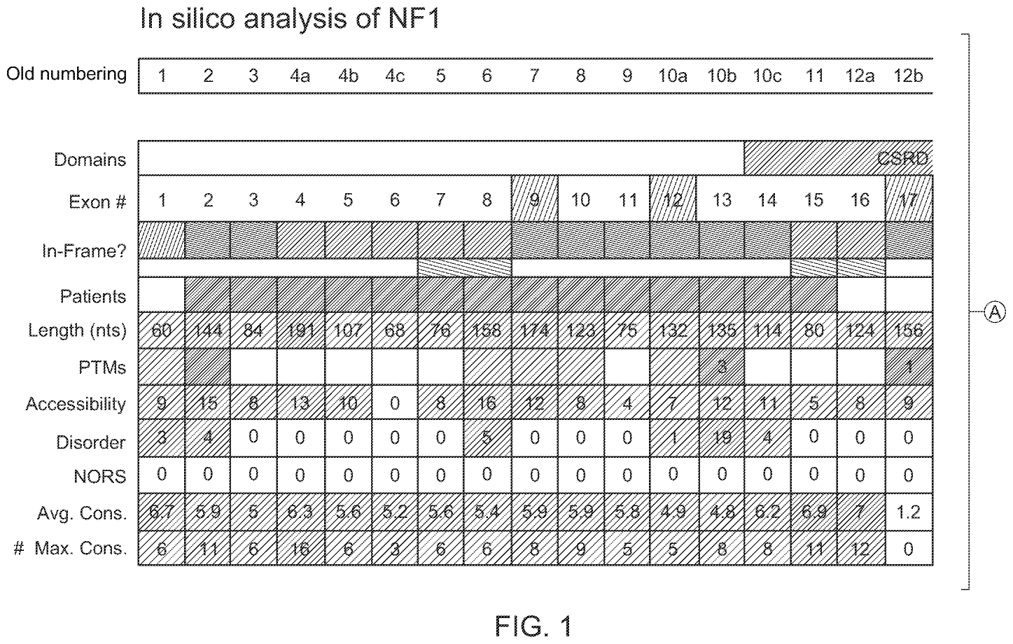

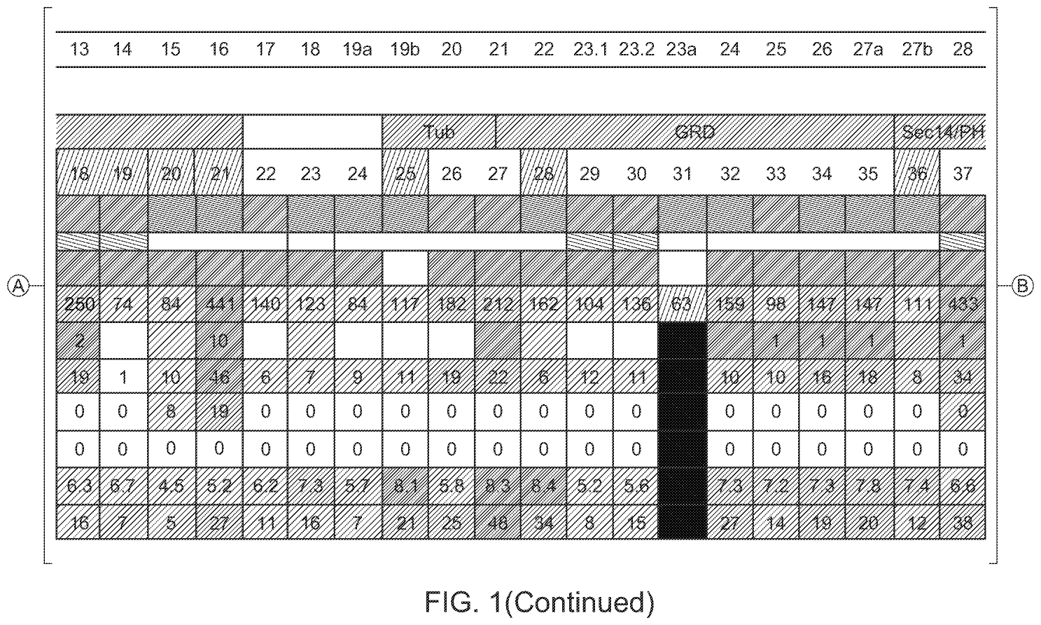

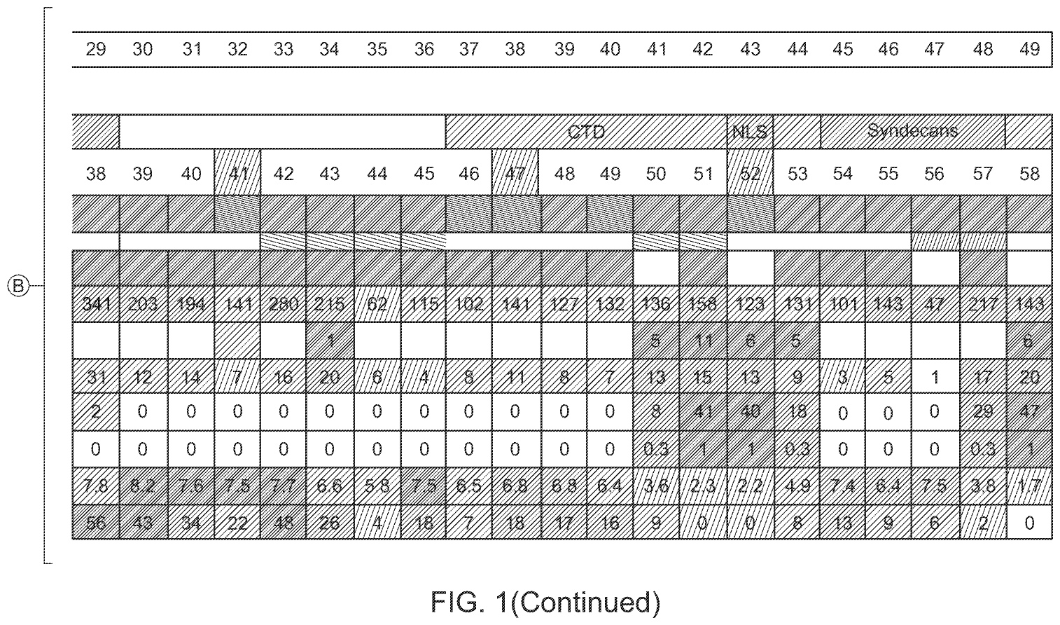

A variety of mutations in the NF1 gene are known, with offer 3000 variants being documented (Ludwine Messiaen, personal communication; see FIG. 1A for exemplary mutations). Germline missense mutations or small in-frame deletions in the NF1 gene may lead to full-length, but unstable polypeptides or polypeptides with aberrant function (˜18% of NF1 patients). A search of the Leiden Open Variation Database (LOVD) for unique missense variants in NF1 yields 383 variants. Of these variants, 30 indicate that the variant does not affect function, 154 indicate the variant does affect function and the rest have questionable classifications. While primarily characterized by neurofibromas, the NF1 phenotype is diverse and variable, even within the same family with the same pathogenic variant. Individuals with NF1 may develop learning disabilities, macrocephaly, optic glioma, disfigurement, abnormalities of the bone, hypertension, and are at increased risk of developing malignant peripheral nerve sheath tumors (MPNSTs).

Exon skipping therapy may utilize specific ASOs. ASOs are short nucleic acid polymers designed to bind the target pre-mRNA through base pairing in a way that induces altered RNA splicing, thereby causing the cellular splicing machinery to “skip” one or more exons carrying a pathological mutation. The resulting mRNAs are then translated into shortened proteins that in the case of a successful therapy are able to compensate the loss of critical functionality as a consequence of the genetic mutation. Antisense directed gene therapy for exon skipping has been is successfully tested for the treatment of a number of diseases (Siva, et al., Nucleic Acid Ther 24, 69-86), most notably Duchenne muscular dystrophy (DMD) (Jarmin, et al., (2014), Expert Opin Biol Ther 14, 209-230). The FDA recently approved the first exon skipping therapy, Eteplirsen (brand name Exondys 51), for ASO-based exon 51 skipping, while therapies targeting other DMD exons are in clinical trials.

The exon skipping strategy for DMD was developed with the knowledge of patients with large in-frame deletions within the DMD gene that developed Becker muscular dystrophy, a much milder disease phenotype than DMD. Unfortunately, a similar situation does not exist for neurofibromin. It is not obvious which regions, if any, might be removed or skipped while retaining crucial functionality. Further, exon skipping strategies for DMD have not been able to restore full dystrophin function, with the shortened dystrophin messengers producing partially functional protein that are sufficient to stabilize the DMD phenotype. It is unknown how much functional neurofibromin is required to prevent classical NF1 phenotypes from occurring.

The literature indicates that cryptic splice sites created by deep intronic mutations within NF1 can be silenced in vitro (Fernandez-Rodriguez, et al., (2011), Hum Mutat 32, 705-709; Pros, et al., (2009), Hum Mutat 30, 454-462). One manuscript describes using antisense morpholino oligomers to successfully target the newly created 5′ splice sites to restore normal splicing in fibroblasts and lymphocyte cell lines with 3 different deep intronic mutations (c.288+2025T>G, c.5749+332A>G, and c.7908-321C>G). This study showed antisense morpholino oligomers-dependent decrease in Ras-GTP levels, which is consistent with the restoration of neurofibromin function. The second study assessed c.3198-314G>A and noted leakiness of the splicing mechanism that generated a proportion of correctly spliced transcripts and demonstrated correction of the splicing defect by using specific ASOs. While repression of a cryptic intronic splice sites has the distinct advantage that no coding part of neurofibromin is removed, a new ASO therapy must be designed and tested for each mutation.

The present disclosure takes the approach of skipping constitutive exons to affect intragenic NF1 mutations that reside in non-critical regions of neurofibromin. Production of even partially functional neurofibromin could help ameliorate phenotypes. Literature in this area is non-existent. The present disclosure describes the systematic of NF1 in silico, in vitro, and in vivo to determine which, if any, exons might be skipped and retain neurofibromin function. The sequences were first analyzed in silico to make predictions and then evaluated in an in vitro cDNA system. Using the mouse cDNA, full-length functional mNf1 cDNA is expressed and the GRD-related functional activity can be validated by different methods: NF1 levels, GTP-bound Ras levels, p-ERK/ERK levels, and ELK-1 transcriptional activity.

Constitutive exon skipping has the benefit of skipping over any mutation within the region of interest and potentially helping many patients with different mutations, while repression of cryptic splice sites is more limited. Ideally, exon skipping could be used clinically to treat NF1 patients that harbor mutations in non-critical regions of the gene. Production of even partially functional neurofibromin could help ameliorate phenotypes.

In the present disclosure, NF1 in silico, in vitro and in vivo evaluations were used to test the effects of deletion of specific exons on neurofibromin function. In silico analysis suggests 44 exons can be deleted as singletons or as doublets and retain the transcript reading frame. Further in silico characterization including prioritization of exons where there have been no reports of NF1 patients with a given exon skip allowed the development of a panel of exons to test in vitro. A recently developed full-length NF1 cDNA expression system was used for functional studies to help determine the regions of NF1 that can be skipped without loss of essential functionality. cDNAs modeling specific exon deletions for neurofibromin levels and Ras activity through GTP-Ras levels, pERK/ERK ratios, and ELK1 transcriptional activity in neurofibromin null HEK293 cells was also evaluated. The present disclosure identified a number of exons that can be skipped while maintaining significant GRD function in at least two Ras activity assays indicating at least partial functionality. In particular, the present disclosure shows that loss of exons 12, 17, 25, 42, 47, and 52 maintain significant GRD function. Further, exons 18/19, 20 and 28 are critical for GRD function and deletion of exons 20, 41, and 47 leads to significantly lower levels of neurofibromin. Skipping of exons 17 and 52 results in both the highest neurofibromin levels and the most suppression of Ras activity. The effects of deletion of exon 17 (DelE17) in vivo were tested in a nullizygous mouse model to show that this exon is not required for at least partial neurofibromin function. DelE17 results in a viable and fertile mouse providing proof-of-concept that exon 17 is not essential for neurofibromin function and may be targeted for exon skipping therapeutics.

In order to induce exon skipping, ASOs can be designed to target (bind to and mask) exonic splicing enhancer (ESE) sites, while preferably avoiding binding to exonic splicing suppressor (ESS) sites. By targeting ESE sites, the normal slicing mechanisms present in the cells do not recognize splice sites for a given exon and the exon skipped or the number of transcripts containing such exon is reduced. In addition, splice sites created by mutation (for example, an out-of-frame cryptic splice site (CSS) mutation) in the NF1 gene can also be targeted by ASOs. By targeting splice sites created by mutation, such splice sites can be masked such that the normal slicing mechanisms present in the cells do not recognize splice site and the exon can be retained or the number of transcripts containing the exon is increased.

As discussed above, exons 3, 7/8, 9, 10, 11, 12, 14, 17, 18/19, 20, 21, 24, 25, 36, 41, 46, 47, 49, and 52, preferably exons 9, 12, 17, 20, 21, 25, 36, 41, 47, and 52, more preferably exons 9, 12, 17, 25, 41, 47, and 52, and more preferably, exons 17, 47, and 52, are suitable candidates for ASO-mediated exon skipping approach as a treatment for NF1 and NF-1 mediated conditions. In addition, a potential masking of an out-of-frame CSS mutation in NF1 exon 13 is a suitable candidate for ASO-mediated exon retention approach as a treatment for NF1 and NF-1 mediated conditions.

Transcript number ENST00000356175 was used in Ensembl release 94, accessed at http://www.ensembl.org/index.html to obtain the sequences of NF1 exons with surrounding introns. The sequence of each exon with 100 intronic nucleotides flanking on both 5′ and 3′ ends were used in Human Splicing Finder Version 3.1 (accessed at http://www.umd.be/HSF3/HSF.shtml) to identify ESE and ESS motifs in each of the exons.

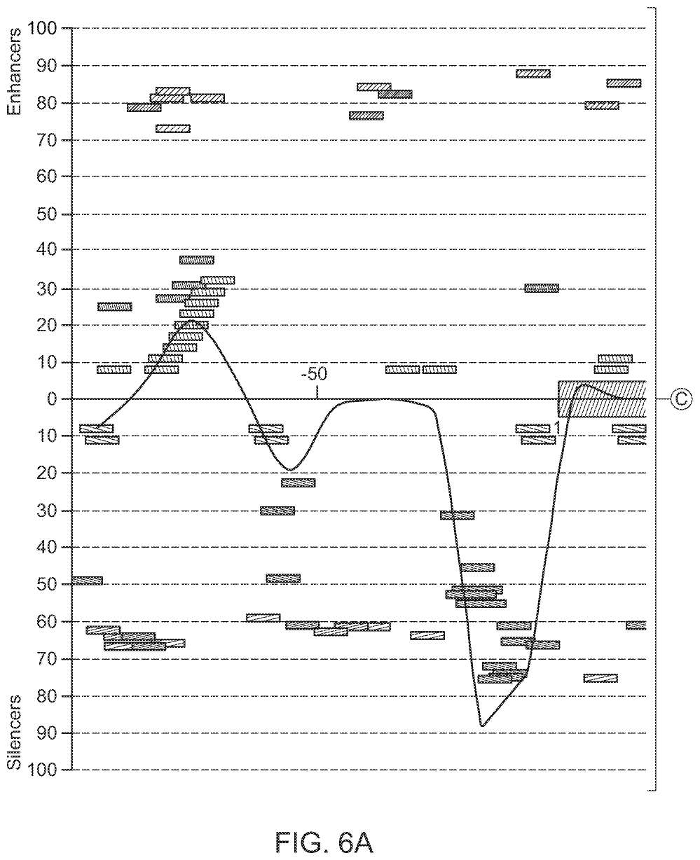

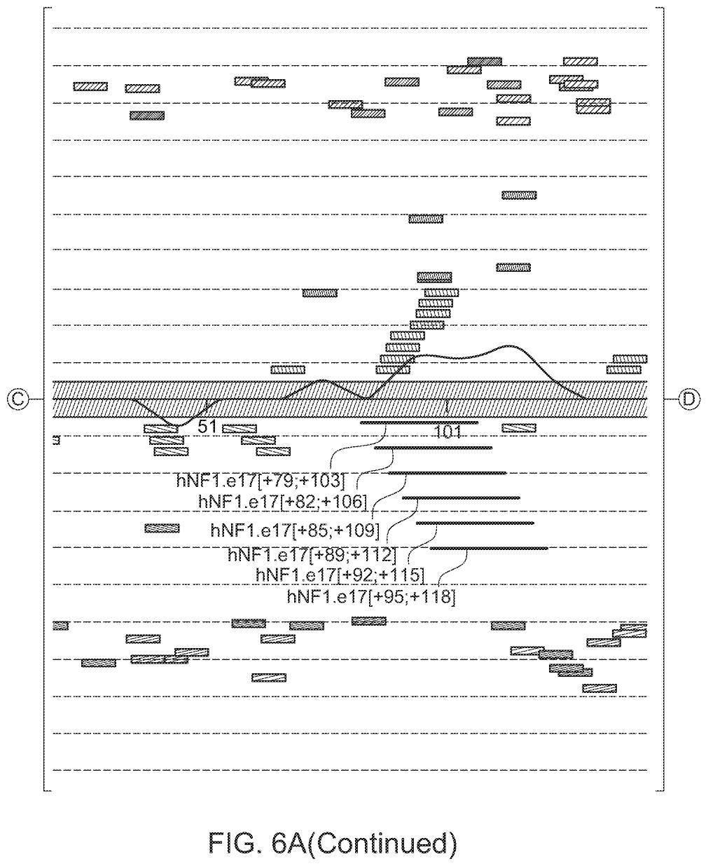

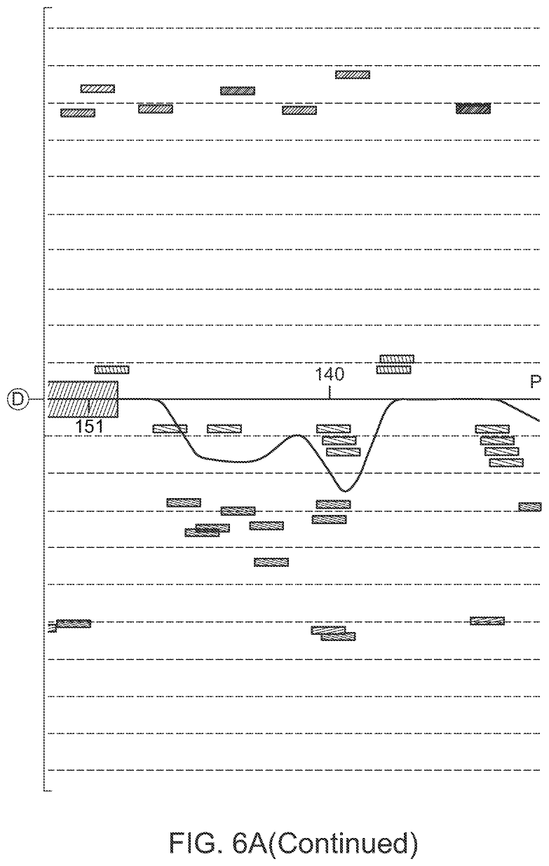

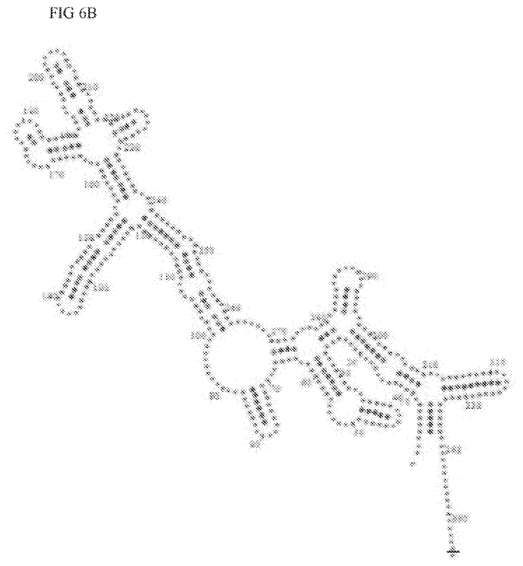

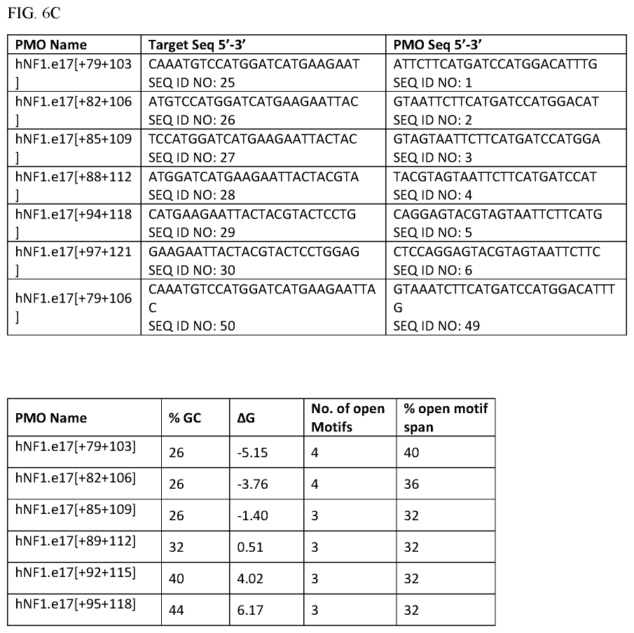

For each of these exons, the NF1 pre-mRNA sequence (typically identified exons with 100 nt of upstream and downstream flanking intron sequence) was interrogated using various bioinformatics tools. Overlapping ASOs were designed to mask ESE motifs in selected exons, namely exons 17, 47, and 52, to induce exon skipping as a proof of concept. For exon 13, ASOs were carefully designed to avoid the ESE motif and mask the CSS mutation to retain exon 13. FIGS. 6A-9A show this analysis for exons 17, 47, 52, and 13, respectively.

Each of the exons and flanking intronic regions, secondary structures were modelled using Visual OMP software, in order to assess the biophysical binding properties of the ASOs to its targets. FIGS. 6B-9B show this analysis for exons 17, 47, 52, and 13, respectively. The target sties for each designed ASO were mapped to the folded pre-mRNA structure and the percentage GC content, ΔG value in kcal mol⁻¹ (overall binding energy), number of target open conformations spanned by each ASO and percentage of ASO nucleotides binding within open conformation of the target were determined. FIGS. 6C-9C show this analysis for exons 17, 47, 52, and 13, respectively.

The capability of designed ASOs binding to the target pre-mRNA sequence was evaluated using RNAup web server (accessed at http://ma.tbi.univie.ac.at/cgibin/RNAWebSuite/RNAup.cgi) to predict the free energies of binding. The ASOs showing the lowest predicted free energy of binding located at the ESE/ESS ratios regions are preferably used. The free energies of binding are summarized in FIGS. 6C-9C for exons 17, 47, 52, and 13, respectively. Off target analysis was performed to make sure that the ASOs do not bind sites other than the targeted sites and create unwanted, off-target effects. The target sequence of each of the ASOs designed were entered in BLASTN (accessed at https://blast.ncbi.nlmr.nih.gO/Blast.cgi?PAGE=ProTeins) and searched against the human genome (Homo sapiens (taxid:9606)) using the default settings. The hits with the E values (an indication of degree of homology) less than 1 were further analyzed to see the sites of off target effects e.g. intronic, exonic, promotor/enhancer region, polyadenylation signals. No predicted off-target effects were identified for the designed oligos.

### Methods

The present disclosure provides for methods of treatment for NF-1 and NF-1 mediated conditions.

In a first embodiment, the method of treatment comprises the step of administering to a subject a therapeutically effective amount of antisense oligonucleotide comprising a sequence that is specifically hybridisable to a target sequence in a NF1 pre-mRNA exon.

In one aspect of this embodiment, the antisense oligonucleotide is identified by the methods described herein. In another aspect of this embodiment, the antisense oligonucleotide contains 20-60 subunits, preferably 20-35 subunits. In another aspect of the first embodiment, the antisense oligonucleotide is specifically hybridisable with one of exons 3, 7/8, 9, 10, 11, 12, 13, 14, 17, 18/19, 20, 21, 24, 25, 36, 41, 46, 47, 49, and 52, preferably exons 9, 12, 13, 17, 20, 21, 25, 36, 41, 47, and 52, more preferably exons 9, 12, 13, 17, 25, 41, 47, and 52, and more preferably, exons 17, 47, and 52. In another aspect of the first embodiment, the antisense oligonucleotide comprises a sequence selected from the group consisting SEQ ID NOS: 1-24, 49, 51, or 53.

In a second embodiment, the method of treatment comprises the step of administering to a subject a therapeutically effective amount of antisense oligonucleotide comprising a sequence that is specifically hybridisable to a target sequence within exon 17 of a NF1 pre-mRNA.

In one aspect of this embodiment, the antisense oligomer comprises a sequence selected from SEQ ID NOS: 1-6 or 49. In another aspect of this embodiment, the antisense oligonucleotide contains 20-60 subunits, preferably 20-35 subunits. In another aspect of this embodiment, a suitable target sequence from exon 17 includes, but is not limited to, SEQ ID NOS: 25-30 or 50 or a continuous stretch of at least 20 nucleotides within SEQ ID NO: 57. Preferably, the target sequence comprises an ESE. In another aspect of this embodiment, the subject is determined to have a mutation in exon 17 that causes at least in part, or is suspected of causing, at least in part, NF1 or an NF1-mediated condition.

In a third embodiment, the method of treatment comprises the step of administering to a subject a therapeutically effective amount of antisense oligonucleotide comprising a sequence that is specifically hybridisable to a target sequence within exon 47 of a NF1 pre-mRNA.

In one aspect of this embodiment, the antisense oligomer comprises a sequence selected from SEQ ID NOS: 7-12 or 51. In another aspect of this embodiment, the antisense oligonucleotide contains 20-60 subunits, preferably 20-35 subunits. In another aspect of this embodiment, a suitable target sequence from exon 47 includes, but is not limited to, SEQ ID NOS: 31-36 or 52 or a continuous stretch of at least 20 nucleotides within SEQ ID NO: 63. Preferably, the target sequence comprises an ESE. In another aspect of this embodiment, the subject is determined to have a mutation in exon 47 that causes at least in part, or is suspected of causing, at least in part, NF1 or an NF1-mediated condition.

In a fourth embodiment, the method of treatment comprises the step of administering to a subject a therapeutically effective amount of antisense oligonucleotide comprising a sequence that is specifically hybridisable to a target sequence within exon 52 of a NF1 pre-mRNA.

In one aspect of this embodiment, the antisense oligomer comprises a sequence selected from SEQ ID NOS: 13-18 or 53. In another aspect of this embodiment, the antisense oligonucleotide contains 20-60 subunits, preferably 20-35 subunits. In another aspect of this embodiment, a suitable target sequence from exon 52 includes, but is not limited to, SEQ ID NOS: 37-42 or 54 or a continuous stretch of at least 20 nucleotides within SEQ ID NO: 64. Preferably, the target sequence comprises an ESE. In another aspect of this embodiment, the subject is determined to have a mutation in exon 52 that causes at least in part, or is suspected of causing, at least in part, NF1 or an NF1-mediated condition.

In a fifth embodiment, the method of treatment comprises the step of administering to a subject a therapeutically effective amount of antisense oligonucleotide comprising a sequence that is specifically hybridisable to a target sequence within exon 13 of a NF1 pre-mRNA.

In one aspect of this embodiment, the antisense oligomer comprises a sequence selected from SEQ ID NOS: 19-24. In another aspect of this embodiment, the antisense oligonucleotide contains 20-60 subunits, preferably 20-35 subunits. In another aspect of this embodiment, a suitable target sequence from exon 13 includes, but is not limited to, SEQ ID NOS: 43-48 or a continuous stretch of at least 20 nucleotides within SEQ ID NO: 65. Preferably, the target sequence comprises a cryptic splice site.

In any of the foregoing embodiments or aspects, the antisense oligonucleotide is identified by the methods described herein. In any of the foregoing embodiments or aspects, the antisense oligonucleotide is substantially uncharged. In any of the foregoing embodiments or aspects, the antisense oligonucleotide is a phosphorodiamidate morpholino oligomer (PMO) or comprises one or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the foregoing embodiments or aspects, the antisense oligonucleotide is interspersed with linkages that are positively charged at physiological pH, where the total number of positively charged linkages is between 1 and no more than half of the total number of linkages. In any of the foregoing embodiments or aspects, the antisense oligonucleotide is substantially uncharged. In any of the foregoing embodiments or aspects, the ASO may have ΔG value between 5 and −1 kcal mol⁻¹, −1 and −11 kcal mol⁻¹, −2 and −10 kcal mol⁻¹, −3, and −9 kcal mol⁻¹, −4 to −8 kcal mol⁻¹, or −5 to −7 kcal mol⁻¹.

In any of the foregoing embodiments or aspects, the subject is determined to have an intragenic NF1 mutation. In any of the foregoing embodiments or aspects, the subject is determined to be in need of treatment. In any of the foregoing embodiments or aspects, the subject is determined to have NF1 or an NF1-mediated condition (for example, through genetic testing). In any of the foregoing embodiments or aspects, the subject is suspected to have NF1 or an NF1-mediated condition (for example, through a familial history). In any of the foregoing embodiments or aspects, the method further comprises administering to the subject a second therapeutic agent useful in the treatment of NF1 or an NF1-mediated condition. In one embodiment, such additional therapeutic agent inhibits the activation of a Ras polypeptide or a polypeptide activated by a RAS polypeptide, such as but not limited to, Raf. MEK1/2, ERK1/2, Elk-1, elf-4E, PI3K, and Akt/PKB.

The present disclosure provides for methods of exon skipping whereby one or more exons of the NF-1 gene are skipped. Such methods of exon skipping may be used for the treatment of NF-1 and NF-1 mediated conditions.

In a first embodiment, the method of exon skipping comprises the step of administering to a subject a therapeutically effective amount of antisense oligonucleotide comprising a sequence that is specifically hybridisable to a target sequence in a NF1 pre-mRNA exon.

In one aspect of this embodiment, the antisense oligonucleotide is identified by the methods described herein. In another aspect of this embodiment, the antisense oligonucleotide contains 20-60 subunits, preferably 20-35 subunits. In another aspect of the first embodiment, the antisense oligonucleotide is specifically hybridisable with one of exons 3, 7/8, 9, 10, 11, 12, 13, 14, 17, 18/19, 20, 21, 24, 25, 36, 41, 46, 47, 49, and 52, preferably exons 9, 12, 13, 17, 20, 21, 25, 36, 41, 47, and 52, more preferably exons 9, 12, 13, 17, 25, 41, 47, and 52, and more preferably, exons 17, 47, and 52. In another aspect of the first embodiment, the antisense oligonucleotide comprises a sequence selected from the group consisting SEQ ID NOS: 1-24, 49, 51, or 53.

In a second embodiment, the method of exon skipping comprises the step of administering to a subject a therapeutically effective amount of antisense oligonucleotide comprising a sequence that is specifically hybridisable to a target sequence within exon 17 of a NF1 pre-mRNA.

In one aspect of this embodiment, the antisense oligomer comprises a sequence selected from SEQ ID NOS: 1-6 or 49. In another aspect of this embodiment, the antisense oligonucleotide contains 20-60 subunits, preferably 20-35 subunits. In another aspect of this embodiment, a suitable target sequence from exon 17 includes, but is not limited to, SEQ ID NOS: 25-30 or 50 or a continuous stretch of at least 20 nucleotides within SEQ ID NO: 57. Preferably, the target sequence comprises an ESE. In another aspect of this embodiment, the subject is determined to have a mutation in exon 17 that causes at least in part, or is suspected of causing, at least in part, NF1 or an NF1-mediated condition.

In a third embodiment, the method of exon skipping comprises the step of administering to a subject a therapeutically effective amount of antisense oligonucleotide comprising a sequence that is specifically hybridisable to a target sequence within exon 47 of a NF1 pre-mRNA.

In one aspect of this embodiment, the antisense oligomer comprises a sequence selected from SEQ ID NOS: 7-12 or 51. In another aspect of this embodiment, the antisense oligonucleotide contains 20-60 subunits, preferably 20-35 subunits. In another aspect of this embodiment, a suitable target sequence from exon 47 includes, but is not limited to, SEQ ID NOS: 31-36 or 52 or a continuous stretch of at least 20 nucleotides within SEQ ID NO: 63. Preferably, the target sequence comprises an ESE. In another aspect of this embodiment, the subject is determined to have a mutation in exon 47 that causes at least in part, or is suspected of causing, at least in part, NF1 or an NF1-mediated condition.

In a fourth embodiment, the method of exon skipping comprises the step of administering to a subject a therapeutically effective amount of antisense oligonucleotide comprising a sequence that is specifically hybridisable to a target sequence within exon 52 of a NF1 pre-mRNA.

In one aspect of this embodiment, the antisense oligomer comprises a sequence selected from SEQ ID NOS: 13-18 or 53. In another aspect of this embodiment, the antisense oligonucleotide contains 20-60 subunits, preferably 20-35 subunits. In another aspect of this embodiment, a suitable target sequence from exon 52 includes, but is not limited to, SEQ ID NOS: 37-42 or 54 or a continuous stretch of at least 20 nucleotides within SEQ ID NO: 64. Preferably, the target sequence comprises an ESE. In another aspect of this embodiment, the subject is determined to have a mutation in exon 52 that causes at least in part, or is suspected of causing, at least in part, NF1 or an NF1-mediated condition.

In any of the foregoing embodiments or aspects, the antisense oligonucleotide is identified by the methods described herein. In any of the foregoing embodiments or aspects, the antisense oligonucleotide is substantially uncharged. In any of the foregoing embodiments or aspects, the antisense oligonucleotide is a phosphorodiamidate morpholino oligomer (PMO) or comprises one or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the foregoing embodiments or aspects, the antisense oligonucleotide is interspersed with linkages that are positively charged at physiological pH, where the total number of positively charged linkages is between 1 and no more than half of the total number of linkages. In any of the foregoing embodiments or aspects, the antisense oligonucleotide is substantially uncharged. In any of the foregoing embodiments or aspects, the ASO may have ΔG value between 5 and −1 kcal mol⁻¹, −1 and −11 kcal mol⁻¹, −2 and 30 −10 kcal mol⁻¹, −3, and −9 kcal mol⁻¹, −4 to −8 kcal mol⁻¹, or −5 to −7 kcal mol⁻¹.

In any of the foregoing embodiments or aspects, the subject is determined to have an intragenic NF1 mutation and/or the exon skipped has an intragenic mutation. In any of the foregoing embodiments or aspects, the subject is determined to be in need of treatment. In any of the foregoing embodiments or aspects, the subject is determined to have NF1 or an NF1-mediated condition (for example, through genetic testing). In any of the foregoing embodiments or aspects, the subject is suspected to have NF1 or an NF1-mediated condition (for example, through a familial history). In any of the foregoing embodiments or aspects, the method further comprises administering to the subject a second therapeutic agent useful in the treatment of NF1 or an NF1-mediated condition. In one embodiment, such additional therapeutic agent inhibits the activation of a Ras polypeptide or a polypeptide activated by a RAS polypeptide, such as but not limited to, Raf. MEK1/2, ERK1/2, Elk-1, elf-4E, PI3K, and Akt/PKB.

The present disclosure provides for methods of exon retention whereby one or more exons of the NF-1 gene that subject to aberrant splicing in at least some of the NF1 pre-mRNA are retained. Such methods of exon retention may be used for the treatment of NF-1 and NF-1 mediated conditions.

In a first embodiment, the method of exon retention comprises the step of administering to a subject a therapeutically effective amount of antisense oligonucleotide comprising a sequence that is specifically hybridisable to a target sequence within exon 13 of a NF1 pre-mRNA.

In one aspect of this embodiment, the antisense oligomer comprises a sequence selected from SEQ ID NOS: 19-24. In another aspect of this embodiment, the antisense oligonucleotide contains 20-60 subunits, preferably 20-35 subunits. In another aspect of this embodiment, a suitable target sequence from exon 13 includes, but is not limited to, SEQ ID NOS: 43-48 or a continuous stretch of at least 20 nucleotides within SEQ ID NO: 65. Preferably, the target sequence comprises a cryptic splice site.

In any of the foregoing embodiments or aspects, the antisense oligonucleotide is identified by the methods described herein. In any of the foregoing embodiments or aspects, the antisense oligonucleotide is substantially uncharged. In any of the foregoing embodiments or aspects, the antisense oligonucleotide is a phosphorodiamidate morpholino oligomer (PMO) or comprises one or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the foregoing embodiments or aspects, the antisense oligonucleotide is interspersed with linkages that are positively charged at physiological pH, where the total number of positively charged linkages is between 1 and no more than half of the total number of linkages. In any of the foregoing embodiments or aspects, the antisense oligonucleotide is substantially uncharged. In any of the foregoing embodiments or aspects, the ASO may have ΔG value between 5 and −1 kcal mol⁻¹, −1 and −11 kcal mol⁻¹, −2 and −10 kcal mol⁻¹, −3, and −9 kcal mol⁻¹, −4 to −8 kcal mol⁻¹, or −5 to −7 kcal mol⁻¹.

In any of the foregoing embodiments or aspects, the subject is determined to be in need of treatment. In any of the foregoing embodiments or aspects, the subject is determined to have NF1 or an NF1-mediated condition (for example, through genetic testing). In any of the foregoing embodiments or aspects, the subject is suspected to have NF1 or an NF1-mediated condition (for example, through a familial history). In any of the foregoing embodiments or aspects, the method further comprises administering to the subject a second therapeutic agent useful in the treatment of NF1 or an NF1-mediated condition. In one embodiment, such additional therapeutic agent inhibits the activation of a Ras polypeptide or a polypeptide activated by a RAS polypeptide, such as but not limited to, Raf. MEK1/2, ERK1/2, Elk-1, elf-4E, PI3K, and Akt/PKB.

The present disclosure provides for methods for treating a subject suffering from a disease or condition associated with a mutation in a NF1 gene encoding a neurofibromin polypeptide, the method comprising administering to a subject a therapeutically effective amount of an antisense oligonucleotide comprising a sequence that is specifically hybridisable to a target sequence in a NF1 pre-mRNA exon.

In a first embodiment, the method of treatment comprises the step of administering to a subject a therapeutically effective amount of antisense oligonucleotide comprising a sequence that is specifically hybridisable to a target sequence in a NF1 pre-mRNA exon.

In one aspect of this embodiment, the antisense oligonucleotide is identified by the methods described herein. In another aspect of this embodiment, the antisense oligonucleotide contains 20-60 subunits, preferably 20-35 subunits. In another aspect of the first embodiment, the antisense oligonucleotide is specifically hybridisable with one of exons 3, 7/8, 9, 10, 11, 12, 13, 14, 17, 18/19, 20, 21, 24, 25, 36, 41, 46, 47, 49, and 52, preferably exons 9, 12, 13, 17, 20, 21, 25, 36, 41, 47, and 52, more preferably exons 9, 12, 13, 17, 25, 41, 47, and 52, and more preferably, exons 17, 47, and 52. In another aspect of the first embodiment, the antisense oligonucleotide comprises a sequence selected from the group consisting SEQ ID NOS: 1-24, 49, 51, or 53.

In a second embodiment, the method of treatment comprises the step of administering to a subject a therapeutically effective amount of antisense oligonucleotide comprising a sequence that is specifically hybridisable to a target sequence within exon 17 of a NF1 pre-mRNA.

In one aspect of this embodiment, the antisense oligomer comprises a sequence selected from SEQ ID NOS: 1-6 or 49. In another aspect of this embodiment, the antisense oligonucleotide contains 20-60 subunits, preferably 20-35 subunits. In another aspect of this embodiment, a suitable target sequence from exon 17 includes, but is not limited to, SEQ ID NOS: 25-30 or 50 or a continuous stretch of at least 20 nucleotides within SEQ ID NO: 57. Preferably, the target sequence comprises an ESE. In another aspect of this embodiment, the subject is determined to have a mutation in exon 17 that causes at least in part, or is suspected of causing, at least in part, NF1 or an NF1-mediated condition.

In a third embodiment, the method of treatment comprises the step of administering to a subject a therapeutically effective amount of antisense oligonucleotide comprising a sequence that is specifically hybridisable to a target sequence within exon 47 of a NF1 pre-mRNA.

In one aspect of this embodiment, the antisense oligomer comprises a sequence selected from SEQ ID NOS: 7-12 or 51. In another aspect of this embodiment, the antisense oligonucleotide contains 20-60 subunits, preferably 20-35 subunits. In another aspect of this embodiment, a suitable target sequence from exon 47 includes, but is not limited to, SEQ ID NOS: 31-36 or 52 or a continuous stretch of at least 20 nucleotides within SEQ ID NO: 63. Preferably, the target sequence comprises an ESE. In another aspect of this embodiment, the subject is determined to have a mutation in exon 47 that causes at least in part, or is suspected of causing, at least in part, NF1 or an NF1-mediated condition.

In a fourth embodiment, the method of treatment comprises the step of administering to a subject a therapeutically effective amount of antisense oligonucleotide comprising a sequence that is specifically hybridisable to a target sequence within exon 52 of a NF1 pre-mRNA.

In one aspect of this embodiment, the antisense oligomer comprises a sequence selected from SEQ ID NOS: 13-18 or 53. In another aspect of this embodiment, the antisense oligonucleotide contains 20-60 subunits, preferably 20-35 subunits. In another aspect of this embodiment, a suitable target sequence from exon 52 includes, but is not limited to, SEQ ID NOS: 37-42 or 54 or a continuous stretch of at least 20 nucleotides within SEQ ID NO: 64. Preferably, the target sequence comprises an ESE. In another aspect of this embodiment, the subject is determined to have a mutation in exon 52 that causes at least in part, or is suspected of causing, at least in part, NF1 or an NF1-mediated condition.

In a fifth embodiment, the method of treatment comprises the step of administering to a subject a therapeutically effective amount of antisense oligonucleotide comprising a sequence that is specifically hybridisable to a target sequence within exon 13 of a NF1 pre-mRNA.

In one aspect of this embodiment, the antisense oligomer comprises a sequence selected from SEQ ID NOS: 19-24. In another aspect of this embodiment, the antisense oligonucleotide contains 20-60 subunits, preferably 20-35 subunits. In another aspect of this embodiment, a suitable target sequence from exon 13 includes, but is not limited to, SEQ ID NOS: 43-48 or a continuous stretch of at least 20 nucleotides within SEQ ID NO: 65. Preferably, the target sequence comprises a cryptic splice site.

In any of the foregoing embodiments or aspects, the antisense oligonucleotide is identified by the methods described herein. In any of the foregoing embodiments or aspects, the antisense oligonucleotide is substantially uncharged. In any of the foregoing embodiments or aspects, the antisense oligonucleotide is a phosphorodiamidate morpholino oligomer (PMO) or comprises one or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the foregoing embodiments or aspects, the antisense oligonucleotide is interspersed with linkages that are positively charged at physiological pH, where the total number of positively charged linkages is between 1 and no more than half of the total number of linkages. In any of the foregoing embodiments or aspects, the antisense oligonucleotide is substantially uncharged. In any of the foregoing embodiments or aspects, the ASO may have ΔG value between 5 and −1 kcal mol⁻¹, −1 and −11 kcal mol⁻¹, −2 and −10 kcal mol⁻¹, −3, and −9 kcal mol⁻¹, −4 to −8 kcal mol⁻¹, or −5 to −7 kcal mol⁻¹.

In any of the foregoing embodiments or aspects, the subject is determined to be in need of treatment. In any of the foregoing embodiments or aspects, the subject is determined to have NF1 or an NF1-mediated condition (for example, through genetic testing). In any of the foregoing embodiments or aspects, the subject is suspected to have NF1 or an NF1-mediated condition (for example, through a familial history). In any of the foregoing embodiments or aspects, the method further comprises administering to the subject a second therapeutic agent useful in the treatment of NF1 or an NF1-mediated condition. In one embodiment, such additional therapeutic agent inhibits the activation of a Ras polypeptide or a polypeptide activated by a RAS polypeptide, such as but not limited to, Raf. MEK1/2, ERK1/2, Elk-1, elf-4E, PI3K, and Akt/PKB.

In any of the foregoing embodiments or aspects, the disease or condition associated with a mutation in a NF1 gene is selected from the group consisting of breast cancer, leukemia, colorectal cancer, brain tumors, adrenal gland tumors, muscle tumors, spinal cord tumors, Plexiform neurofibromas, MPNST, soft tissue cancer, optic glioma, and Lisch nodules. In any of the foregoing embodiments or aspects, the disease or condition associated with a mutation in a NF1 gene is selected from the group consisting of cafe au lait spots, neurofibromas, bone deformities, osteoporosis, macrocephaly, high blood pressure, learning disabilities, short stature, and scoliosis.

In any of the foregoing first to fourth embodiments or aspects thereof, the administration results in exon skipping. In any of the foregoing first to fourth embodiments or aspects thereof, the effect of the mutation can be reduced by exon skipping of the exon containing the mutation. Unless otherwise specified in a specific embodiment, such exons include exons 3, 7/8, 9, 10, 11, 12, 14, 17, 18/19, 20, 21, 24, 25, 36, 41, 46, 47, 49, and 52, preferably exons 9, 12, 17, 20, 21, 25, 36, 41, 47, and 52, more preferably exons 9, 12, 17, 25, 41, 47, and 52, and more preferably, exons 17, 47, and 52. In any of the foregoing first to fourth embodiments or aspects thereof, the mutation is an intragenic mutation.

In any of the foregoing first to fourth embodiments or aspects thereof, the administration results in exon skipping of the exon containing the mutation, wherein the mutation is an intragenic mutation. Unless otherwise specified in a specific embodiment, such exons include exons 3, 7/8, 9, 10, 11, 12, 14, 17, 18/19, 20, 21, 24, 25, 36, 41, 46, 47, 49, and 52, preferably exons 9, 12, 17, 20, 21, 25, 36, 41, 47, and 52, more preferably exons 9, 12, 17, 25, 41, 47, and 52, and more preferably, exons 17, 47, and 52.

In the fifth embodiments or aspects thereof, the effect of the mutation can be reduced by exon retention of an exon containing the mutation, such as for example, exon 13.

In any of the foregoing embodiments or aspects, the method may further comprise selecting the antisense oligonucleotide that binds or specifically hybridizes to a target sequence. In any of the foregoing embodiments or aspects, the administration results in an increased production of a functional or biologically active form of the neurofibromin polypeptide.

### Compounds of the Disclosure

The compounds described herein may be used as a prophylactic or therapeutic for the purpose of treatment of a genetic disease, preferably NF1. Accordingly, the present invention provides compounds, including oligonucleotides and ASOs, that bind to or are specifically hybridisable to a target sequence in the NF1 pre-mRNA to induce exon skipping or exon retention as described herein. Such compounds are preferably administered in a therapeutically effective amount in a pharmaceutical composition described herein.

The present disclosure further provides for compounds for use in a method as described herein. This compound preferably comprises, consists of, or consists essentially of, an oligonucleotide, preferably an antisense oligonucleotide (ASO), including an antisense oligoribonucleotide. The compound preferably binds to a target nucleic acid sequence in a cell of a subject, particularly a pre-mRNA or an mRNA. In certain aspects, the compound binds to a target nucleic acid sequence that contains or comprises an exonic splice enhancer (ESE).

The present disclosure demonstrates that particular exons in the NF1 gene may be skipped using the compounds of the present disclosure. Such exons include, but are not limited to, exons 3, 7/8, 9, 10, 11, 12, 14, 17, 18/19, 20, 21, 24, 25, 36, 41, 46, 47, 49, and 52. In a preferred embodiment, such exons are selected from the group consisting of exons 9, 12, 17, 20, 21, 25, 36, 41, 47, and 52. In a more preferred embodiment, such exons are selected from the group consisting of exons 9, 12, 17, 25, 41, 47, and 52. In still a more preferred embodiment, such exons are selected from the group consisting of exons 17, 41, 47, and 52. In a most preferred embodiment, such exons are selected from the group consisting of exons 17, 47, and 52. The compounds that that bind the exons, including the exons in the embodiments above, have a length of 20 to 60 nucleotides, such as at least 20, 25, 30, 35, 40, 45, or 50 nucleotides (but less than 60 nucleotides) or at least 20, 21, 22, 23, 24, 25, 26, 27, 28, 29, 30, 31, 32, or 33 (but less than 40 nucleotides). In certain embodiments, the compounds bind to a continuous stretch of at least 18 nucleotides within said exon. Increasing the length of consecutive nucleotides bound by the compounds is generally associated with a higher binding affinity, although other factors may be involved such as, but not limited to, the thermodynamic, kinetic, or structural characteristics of the hybrid duplex formed by the compound and the target sequence. In one embodiment, a compound described herein binds to a continuous stretch of at least 20, 25, 30, 35, 40, 45, or 50 nucleotides within the exon, but less than or equal to 60 nucleotides. Preferably, a compound described herein binds to a continuous stretch of at least 20, 21, 22, 23, 24, 25, 26, 27, 28, 29, 30, 31, 32, or 33 nucleotides within the exon, but less than or equal to 40 nucleotides.

In one embodiment, an oligonucleotide described herein comprises a sequence that is complementary or specifically hybridisable to a portion of an exon in the NF1 pre-mRNA, where the complementary or specifically hybridisable sequence is at least 50% of the length of the oligonucleotide, at least 60% of the length of the oligonucleotide, at least 70% of the length of the oligonucleotide, at least 80% of the length of the oligonucleotide, at least 90% of the length of the oligonucleotide at least 95% of the length of the oligonucleotide, or at least 98% to 100% of the length of the oligonucleotide.

In one embodiment, “A portion of an exon” as used herein preferably means a stretch of at least 18 consecutive nucleotides of that exon. In a particular embodiment, the length of the complementary or specifically hybridisable part of said oligonucleotide is at least 20 to 60 nucleotides. In a preferred embodiment, the length of the complementary or specifically hybridisable part of said oligonucleotide is at least 20, 21, 22, 23, 24, 25, 26, 27, 28, 29, 30, 31, 32, or 33 nucleotides. Suitable portions of various exons are described herein. In certain embodiments, an oligonucleotide may comprise a sequence that is complementary or specifically hybridisable to part of an exon in the NF1 pre-mRNA as defined herein and an additional flanking sequence(s). Preferably, additional flanking sequences are used to modify the binding of a cellular component, such as, but not limited to, a protein to the oligonucleotide, or to modify a thermodynamic property of the oligonucleotide, such as, but not limited to, binding affinity. In certain aspects of this embodiment, the flanking sequences are complementary to sequences within the exon of the NF1 pre-mRNA, the flanking sequences are complementary to sequences which are not present within the exon of the NF1 pre-mRNA, or a combination of the foregoing. Such flanking sequences may be complementary to sequences comprising, consisting essentially of, or consisting of sequences of an intron of the NF1 pre-mRNA which is adjacent to the exon to be skipped (for example if exon 17 is to be skipped the intron sequence may be intron 17 or 18).

A continuous stretch of nucleotides within an exon of NF1 pre-mRNA may be selected from those sequences described herein. For example, for exons 9, 12, 17, 20, 21, 25, 36, 41, 47, and 52 the continuous stretch of nucleotides may be selected from SEQ ID NOS: 55 to 64, respectively.

In one embodiment, the continuous stretch of nucleotides within exon 17 of NF1 pre-mRNA is selected from the group consisting of: SEQ ID NOS: 25-30, and 50. In another embodiment, the continuous stretch of nucleotides within exon 17 of NF1 pre-mRNA is a sequence of 20, 21, 22, 23, 24, 25, 26, 27, 28, 29, 30, 31, 32, or 33 nucleotides from SEQ ID NO: 57. In another embodiment, the continuous stretch of nucleotides within exon 17 of NF1 pre-mRNA is a sequence of 33 to 39 nucleotides centered on nucleotide 91, 94, 97, 101, 104, or 107 of SEQ ID NO: 57. In another embodiment, the continuous stretch of nucleotides within exon 17 of NF1 pre-mRNA is a sequence of 29 nucleotides centered on nucleotide 91, 94, 97, 101, 104, or 107 of SEQ ID NO: 57. In another embodiment, the continuous stretch of nucleotides within exon 17 of NF1 pre-mRNA is a sequence of 24 nucleotides centered on nucleotide 91, 94, 97, 101, 104, or 107 of SEQ ID NO: 57. In any of the foregoing, the recited nucleic acid sequence contains an ESE. In a preferred embodiment of any of the foregoing, the oligonucleotide specifically hybridisable to the recited nucleotide sequence is selected from the group consisting of SEQ ID NOS: 1-6 and 49.

In one embodiment, the continuous stretch of nucleotides within exon 47 of NF1 pre-mRNA is selected from the group consisting of: SEQ ID NOS: 31-36 and 52. In another embodiment, the continuous stretch of nucleotides within exon 47 of NF1 pre-mRNA is a sequence of 20, 21, 22, 23, 24, 25, 26, 27, 28, 29, 30, 31, 32, or 33 nucleotides from SEQ ID NO: 63. In another embodiment, the continuous stretch of nucleotides within exon 47 of NF1 pre-mRNA is a sequence of 33 to 39 nucleotides centered on nucleotide 47, 50, 53, 88, 91, or 94 of SEQ ID NO: 63. In another embodiment, the continuous stretch of nucleotides within exon 47 of NF1 pre-mRNA is a sequence of 29 nucleotides centered on nucleotide 47, 50, 53, 88, 91, or 94 of SEQ ID NO: 63. In another embodiment, the continuous stretch of nucleotides within exon 47 of NF1 pre-mRNA is a sequence of 24 nucleotides centered on nucleotide 47, 50, 53, 88, 91, or 94 of SEQ ID NO: 63. In any of the foregoing, the recited nucleic acid sequence contains an ESE. In a preferred embodiment of any of the foregoing, the oligonucleotide specifically hybridisable to the recited nucleotide sequence is selected from the group consisting of SEQ ID NOS: 7-12 and 51.

In one embodiment, the continuous stretch of nucleotides within exon 52 of NF1 pre-mRNA is selected from the group consisting of: SEQ ID NOS: 37-42 and 54. In another embodiment, the continuous stretch of nucleotides within exon 52 of NF1 pre-mRNA is a sequence of 20, 21, 22, 23, 24, 25, 26, 27, 28, 29, 30, 31, 32, or 33 nucleotides from SEQ ID NO: 64. In another embodiment, the continuous stretch of nucleotides within exon 52 of NF1 pre-mRNA is a sequence of 33 to 39 nucleotides centered on nucleotide 22, 63, 91, 94, 97, or 100 of SEQ ID NO: 64. In another embodiment, the continuous stretch of nucleotides within exon 52 of NF1 pre-mRNA is a sequence of 29 nucleotides centered on nucleotide 22, 63, 91, 94, 97, or 100 of SEQ ID NO: 64. In another embodiment, the continuous stretch of nucleotides within exon 52 of NF1 pre-mRNA is a sequence of 24 nucleotides centered on nucleotide 22, 63, 91, 94, 97, or 100 of SEQ ID NO: 64. In any of the foregoing, the recited nucleic acid sequence contains an ESE. In a preferred embodiment of any of the foregoing, the oligonucleotide specifically hybridisable to the recited nucleotide sequence is selected from the group consisting of SEQ ID NOS: 13-18 and 53.

As used herein the term “centered on” a particular nucleotide (the reference nucleotide) means that the recited nucleic acid sequence contains the designated number of nucleotides with the reference nucleotide being at the center of the recited nucleic acid sequence. For example, a nucleic acid sequence of 33 nucleotides “centered on” nucleotide 91 of exon 17 has 16 nucleotides 5′ of nucleotide 91 and 16 nucleotides 3′ of nucleotide 91.

It was found that an oligonucleotide that binds to a nucleotide sequence comprising, consisting essentially of, or consisting of a continuous stretch nucleotides as set forth above resulted in skipping of exons 17, 47, and 52.

In one embodiment, an oligonucleotide described herein is capable of interfering with the inclusion of the recited exons of the NF1 pre-mRNA by binding to the recited nucleotide sequence. Methods for screening compound compounds that bind specific nucleotide sequences are for example disclosed in U.S. Pat. No. 6,875,736. In a preferred aspect of this embodiment, the oligonucleotide is an ASO that is specifically hybridisable to the coding strand of the pre-mRNA of NF1, particularly with the nucleotide sequences recited herein. Such ASO may contain one or more nucleotide analogues as disclosed herein in addition to a RNA or DNA residue.

A preferred oligonucleotide of the disclosure, such as, but not limited to, an ASO, comprises a sequence of between 20 and 50 nucleotides or bases, preferably between 20 and 40 nucleotides, preferably between 20 and 35 nucleotides, and more preferably between 20 and 30 nucleotides, such as 20, 21, 22, 23, 24, 25, 26, 27, 28, 29, or 30 nucleotides or bases. A most preferred oligonucleotide comprises a sequence of 25 or 28 nucleotides or bases.

In one embodiment, an oligonucleotide of the disclosure specifically hybridizes to a continuous stretch of at least 20 nucleotides within NF1 pre-mRNA exons 9, 12, 17, 20, 21, 25, 36, 41, 47, 52, or 13 (SEQ ID NOS: 55-64, respectively). In one embodiment, an oligonucleotide of the disclosure specifically hybridizes to a continuous stretch of 20-40 nucleotides, such as 20, 21, 22, 23, 24, 25, 26, 27, 28, 29, or 30 nucleotides, within NF1 pre-mRNA exons 9, 12, 17, 20, 21, 25, 36, 41, 47, 52, or 13. In one embodiment, an oligonucleotide of the disclosure specifically hybridizes to a continuous stretch of at least 20 nucleotides within exons 17, 47, or 52 (SEQ ID NOS: 57, 63, or 64, respectively). In one embodiment, an oligonucleotide of the disclosure specifically hybridizes to a continuous stretch of 20-40 nucleotides, such as 20, 21, 22, 23, 24, 25, 26, 27, 28, 29, or 30 nucleotides, within exons 17, 47, or 52.

In one embodiment, an oligonucleotide of the disclosure comprises, consists essentially of, or consists of, a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 49, SEQ ID NO: 51, or SEQ ID NO: 53. In one embodiment, an oligonucleotide of the disclosure comprises, consists essentially of, or consists of, a sequence selected from SEQ ID NO: 1, SEQ ID NO: 3, SEQ ID NO: 49, SEQ ID NO: 51, or SEQ ID NO: 53.

In one embodiment, an oligonucleotide of the disclosure comprises, consists essentially of, or consists of, a sequence selected from SEQ ID NO: 1, SEQ ID NO: 2, SEQ ID NO: 3, SEQ ID NO: 4, SEQ ID NO: 5, SEQ ID NO: 6, or SEQ ID NO: 49 and specifically hybridizes to a continuous stretch of at least 20 nucleotides (the target sequence) in NF1 exon 17 pre-mRNA. In one aspect of this embodiment, the oligonucleotide is selected from SEQ ID NO: 1, SEQ ID NO: 3, or SEQ ID NO: 49. Representative target sequences from these ASOs are provided in FIG. 6C. In one aspect of this embodiment, the ASOs induce exon skipping of exon 17.

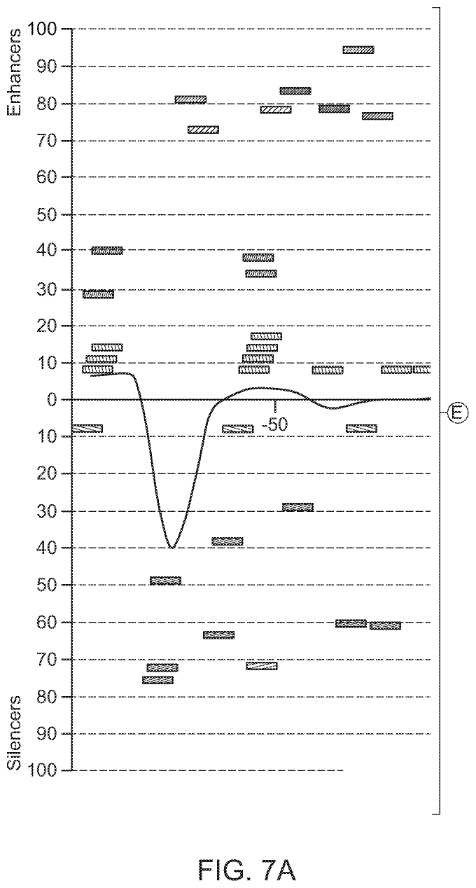

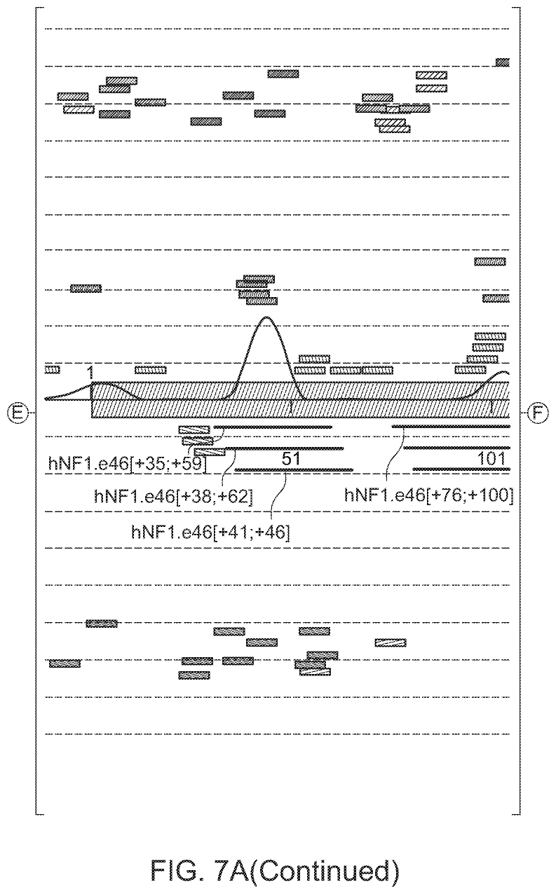

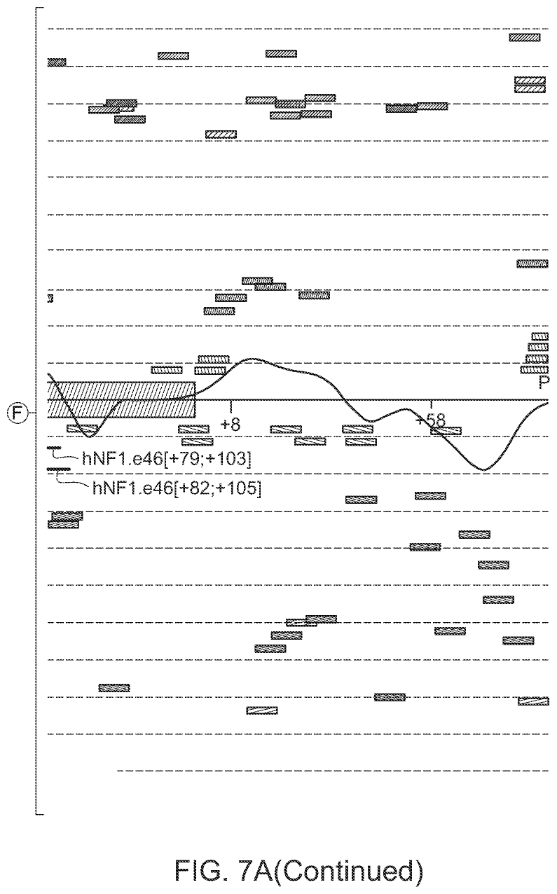

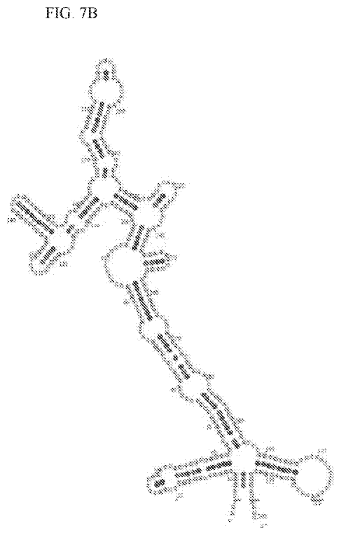

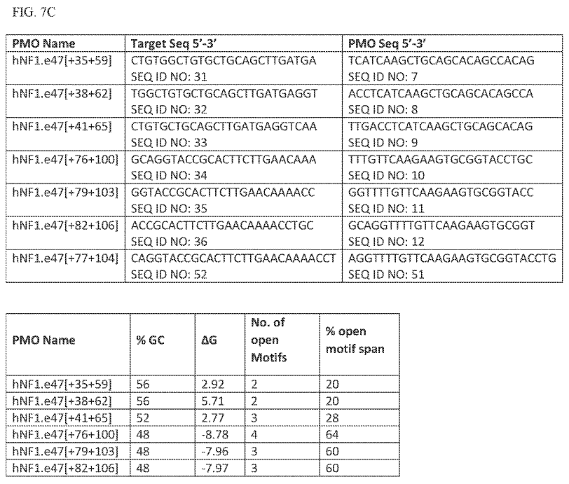

In one embodiment, an oligonucleotide of the disclosure comprises, consists essentially of, or consists of, a sequence selected from SEQ ID NO: 7, SEQ ID NO: 8, SEQ ID NO: 9, SEQ ID NO: 10, SEQ ID NO: 11, SEQ ID NO: 12, SEQ ID NO: 51 and specifically hybridizes to a continuous stretch of at least 20 nucleotides (the target sequence) in NF1 exon 47 pre-mRNA. In one aspect of this embodiment, the oligonucleotide is SEQ ID NO: 51. Representative target sequences from these ASOs are provided in FIG. 7C. In one aspect of this embodiment, the ASOs induce exon skipping of exon 47.

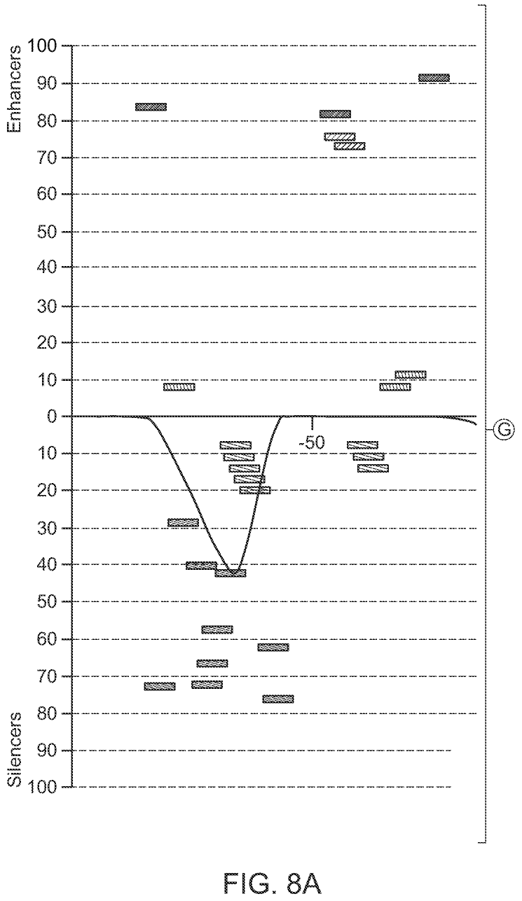

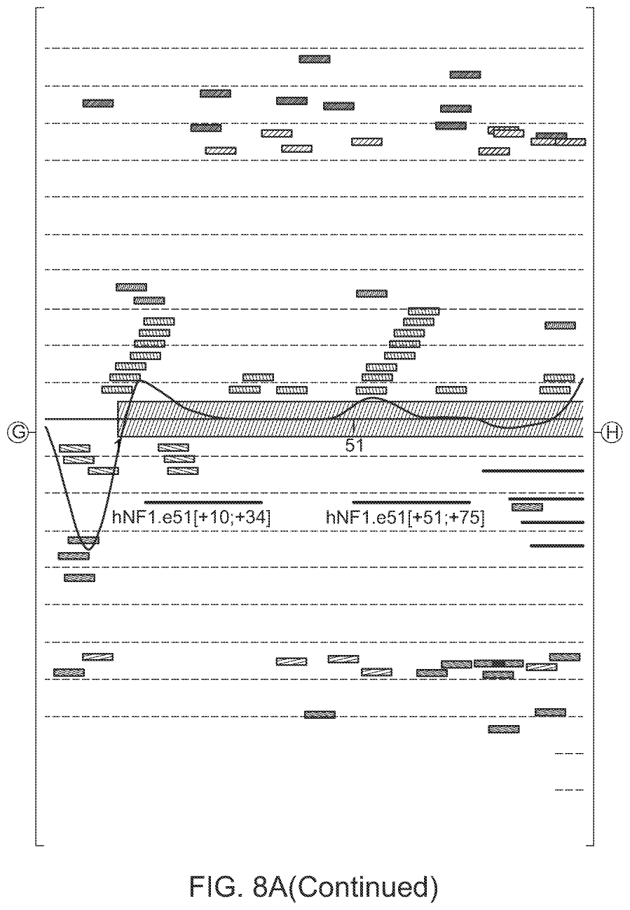

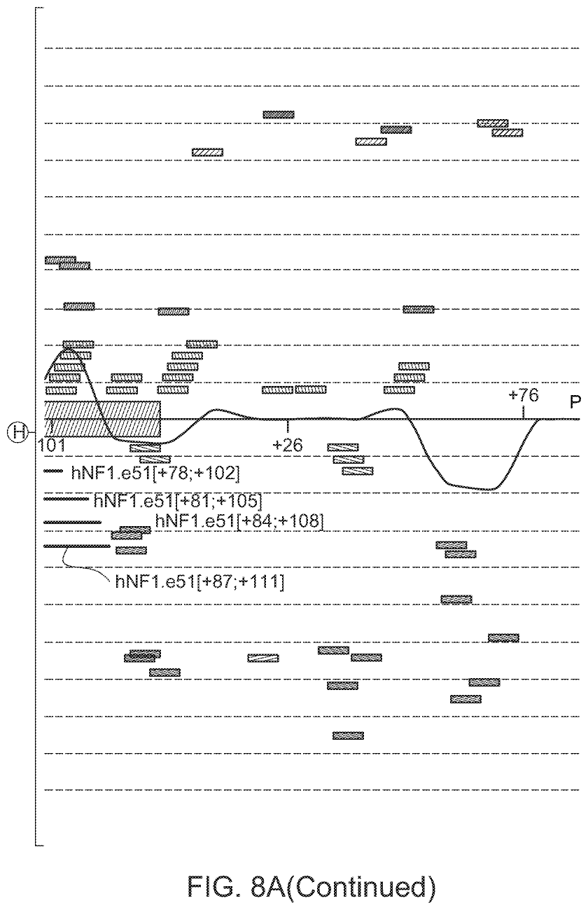

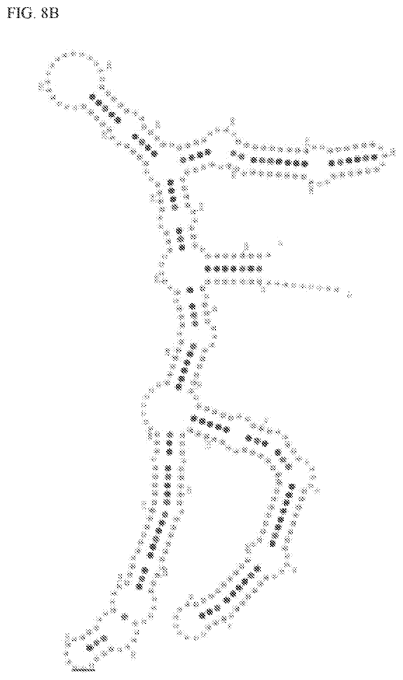

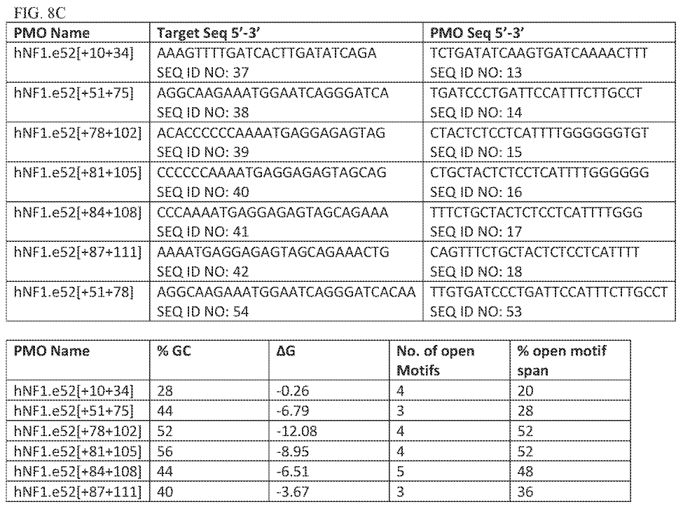

In one embodiment, an oligonucleotide of the disclosure comprises, consists essentially of, or consists of, a sequence selected from SEQ ID NO: 13, SEQ ID NO: 14, SEQ ID NO: 15, SEQ ID NO: 16, SEQ ID NO: 17, SEQ ID NO: 18, or SEQ ID NO: 53 and specifically hybridizes to a continuous stretch of at least 20 nucleotides (the target sequence) in NF1 exon 52 pre-mRNA. In one aspect of this embodiment, the oligonucleotide is SEQ ID NO: 53. Representative target sequences from these ASOs are provided in FIG. 8C. In one aspect of this embodiment, the ASOs induce exon skipping of exon 52.

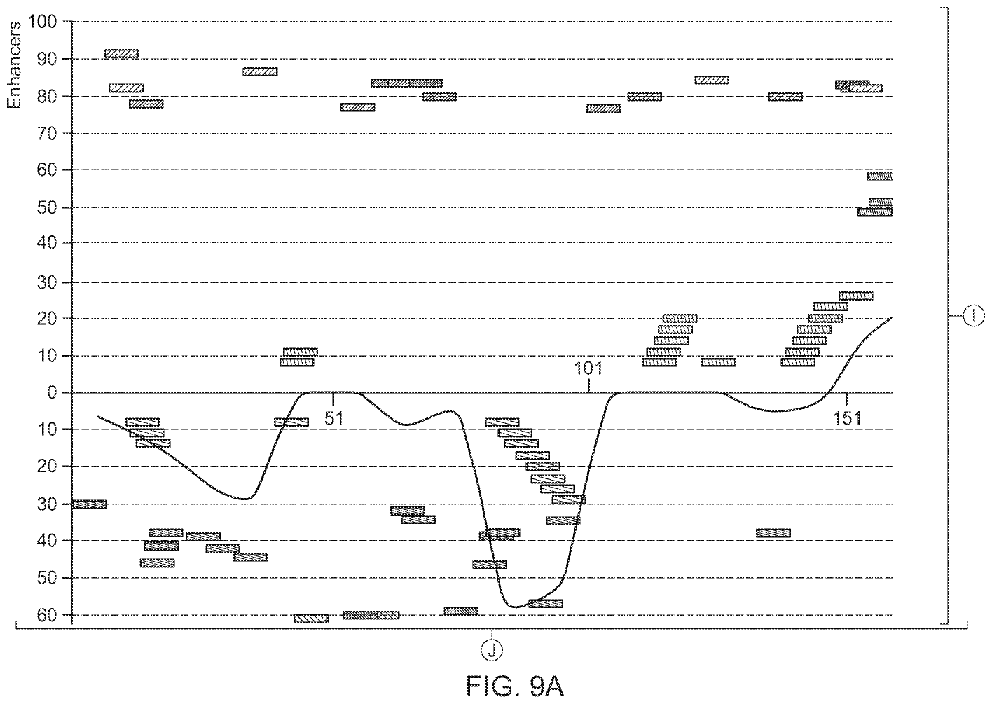

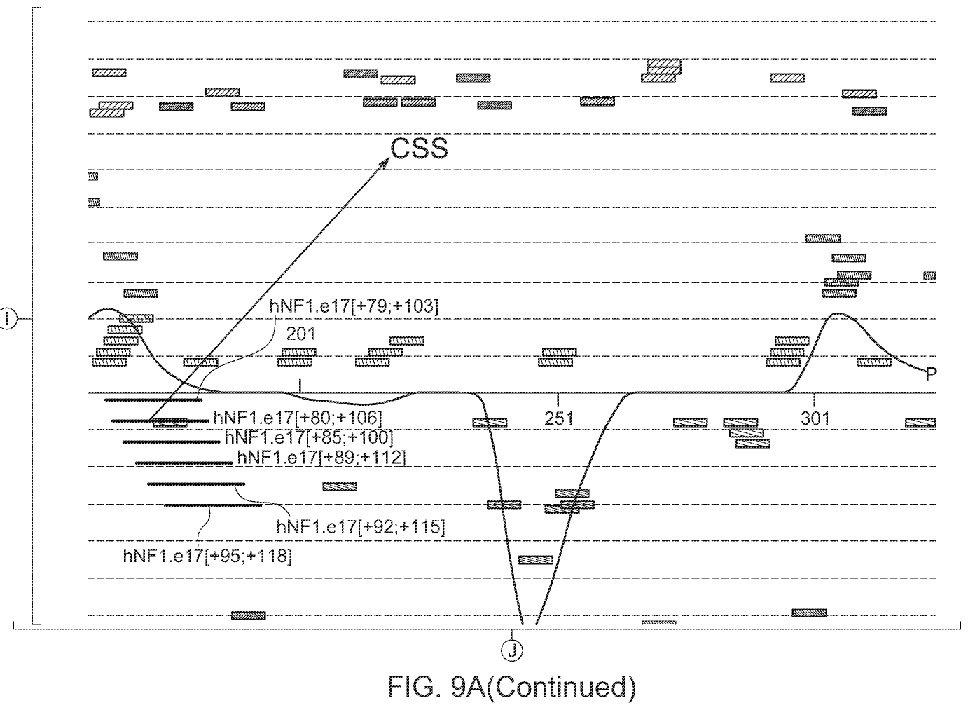

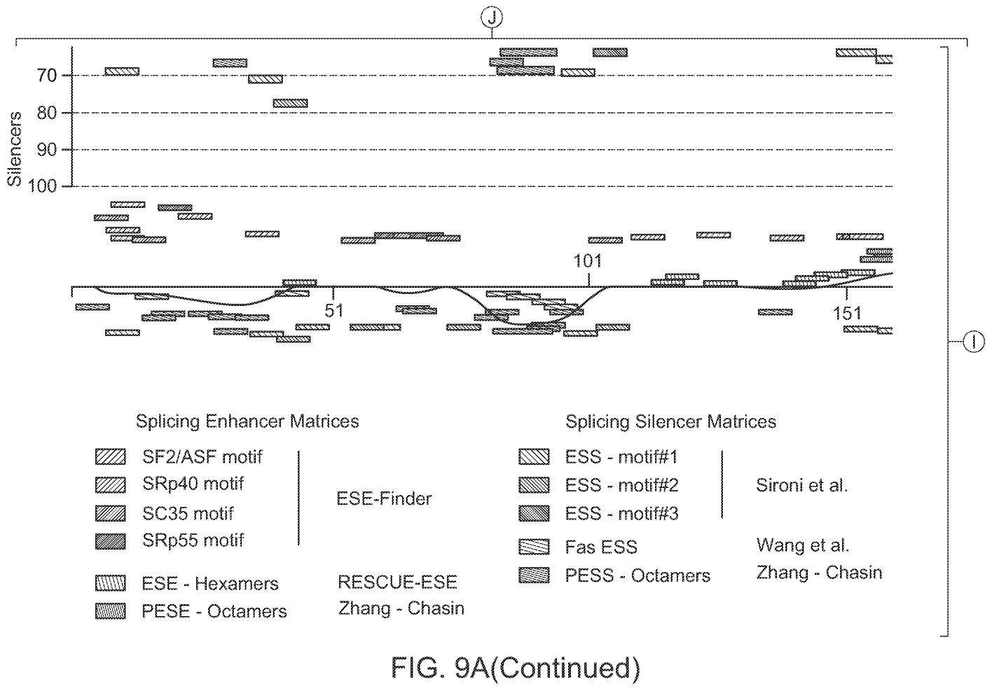

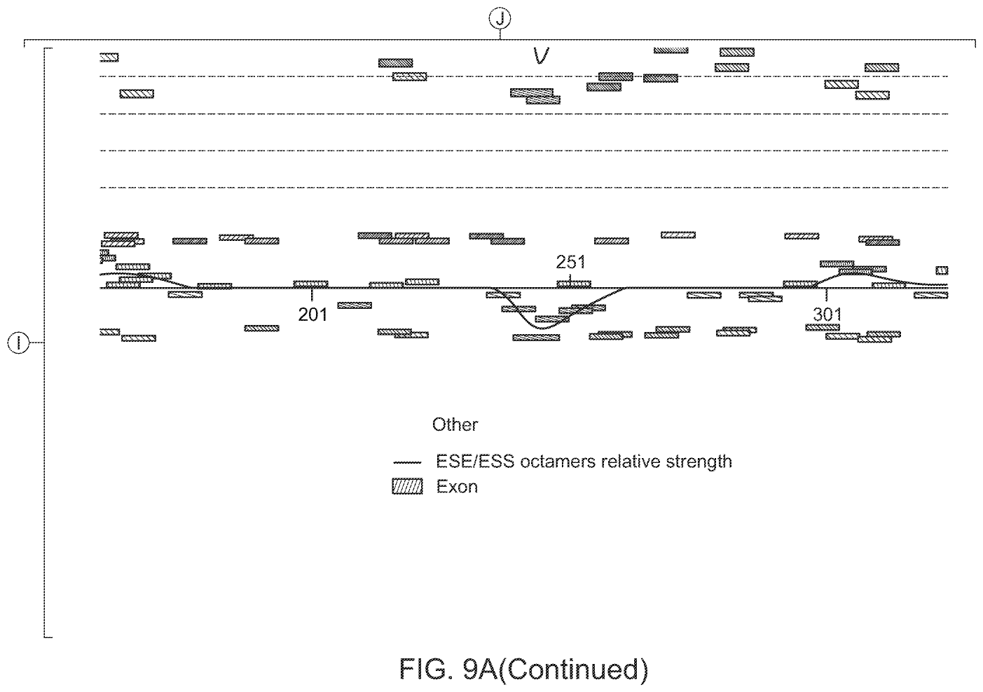

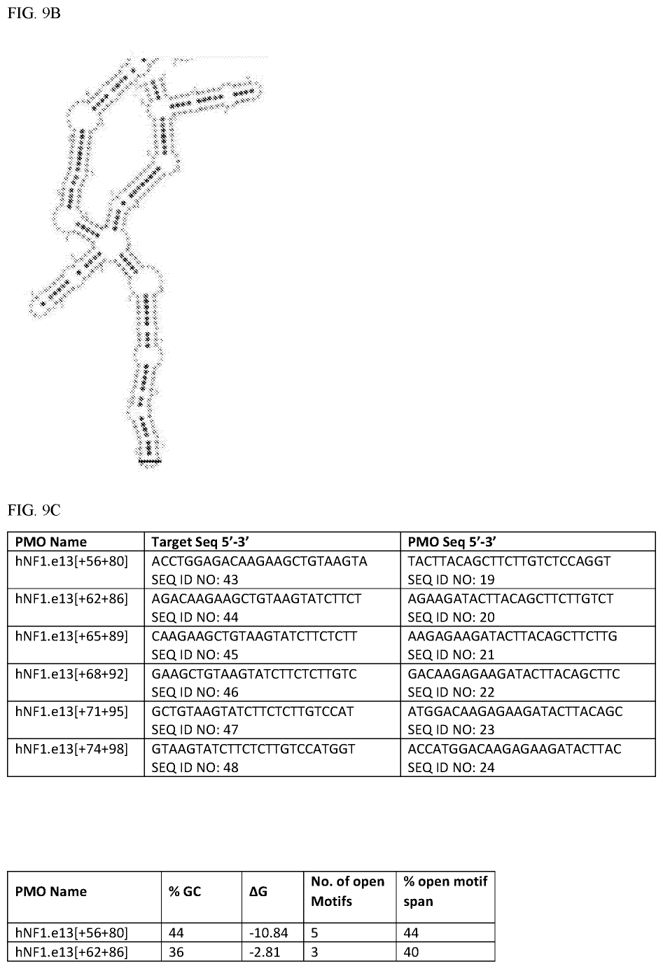

In one embodiment, an oligonucleotide of the disclosure comprises, consists essentially of, or consists of, a sequence selected from SEQ ID NO: 19, SEQ ID NO: 20, SEQ ID NO: 21, SEQ ID NO: 22, SEQ ID NO: 23, or SEQ ID NO: 24 and specifically hybridizes to a continuous stretch of at least 20 nucleotides (the target sequence) in NF1 exon 13 pre-mRNA. Representative target sequences from these ASOs are provided in FIG. 9C. In one aspect of this embodiment, the ASOs induce exon retention of exon 13.

A nucleotide sequence of a compound of the invention may contain a RNA residue, a DNA residue, a nucleotide analogue or equivalent. In preferred embodiment, an oligonucleotide of the disclosure contains at least one residue that is modified to increase nuclease resistance, to increase the affinity of the oligonucleotide for the target nucleotide sequence, or a combination of the foregoing. In a preferred embodiment, an oligonucleotide of the disclosure, such as an ASO, comprises at least one modified nucleotide analogue (i.e., a modified residue), wherein the nucleotide analogue as a modified base, a modified backbone, a non-natural inter-nucleoside linkage, or a combination of any of the foregoing.

In a preferred embodiment, a nucleotide analogue comprises a modified backbone, such as, but not limited to, a morpholino backbone, carbamate backbone, siloxane backbone, sulfide backbone, sulfoxide backbone, sulfone backbone, formacetyl backbone, thioformacetyl backbone, methyleneformacetyl backbone, riboacetyl backbone, alkene containing backbone, sulfamate backbone, sulfonate backbone, sulfonamide backbone, methyleneimino backbone, methylenehydrazino backbone, and amide backbone. Phosphorodiamidate morpholino oligomers are modified backbone oligonucleotides that have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing. Morpholino oligonucleotides have been successfully delivered to tissue culture cells by methods that physically disrupt the cell membrane.

In a preferred embodiment, the linkage between residues in a backbone does not include a phosphorus atom, such as, but not limited to, a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred backbone is a morpholino nucleotide analog, in which the sugar moiety (such as a ribose or deoxyribose sugar) is replaced by a 6-membered morpholino ring. A most preferred nucleotide analog is a morpholino moiety, in which the ribose or deoxyribose sugar is replaced by a 6-membered morpholino ring, and the anionic phosphodiester linkage between adjacent morpholino rings is replaced by a non-ionic phosphorodiamidate linkage resulting in a phosphorodiamidate morpholino oligomer (PMO).

A further preferred nucleotide analogue is a peptide nucleic acid (PNA), having a modified polyamide backbone. PNA-based compounds are true mimics of DNA molecules in is terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA compound contains no charged phosphate groups, PNA-RNA hybrids are usually more stable than RNA-RNA or RNA-DNA hybrids.

In another embodiment, a nucleotide analogue comprises a substitution of at least one of the non-bridging oxygen atoms in the phosphodiester linkage adds significant resistance to nuclease degradation at the cost of a slight destabilization of base-pairing. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue comprises one or more sugar moieties that are mono- or di-substituted at the 2′, 3′ and/or 5′ position with —OH, —F, substituted or unsubstituted, linear or branched lower (C1-C10) alkyl, alkenyl, alkynyl, alkaryl, allyl, aryl, or aralkyl, that may be interrupted by one or more heteroatoms selected from the group consisting of: O-alkyl, S-alkyl, N-alkyl, O-alkenyl, S-alkenyl, N-alkenyl, O-alkynyl, S-alkynyl, N-alkynyl, O-alkyl-O-alkyl, -methoxy, -aminopropoxy, -aminoxy, -aminomethoxyethoxy, -dimethylaminooxyethoxy, and -dimethylaminoethoxyethoxy. The sugar moiety can be a pyranose or a deoxypyranose, preferably a ribose or deoxyribose. Such preferred derivatized sugar moieties comprise locked nucleic acid (LNA), in which the 2-carbon atom is linked to the 3′ or 4′ carbon atom of the sugar ring thereby forming a bicyclic sugar moiety. A preferred LNA comprises 2′-O,4′-C-ethylene-bridged nucleic acid. These substitutions render the nucleotide analogue or equivalent RNase H and nuclease resistant and increase the affinity for the target RNA.

It is understood by a skilled person that it is not necessary for all positions of an oligonucleotide of the disclosure to be modified. In addition, more than one of the aforementioned nucleotide analogues may be incorporated in a single oligonucleotide. In certain embodiments, an oligonucleotide of the disclosure has a single type of nucleotide analogues. In certain embodiments, an oligonucleotide of the disclosure has at least two different types of nucleotide analogues.

A functional equivalent refers to an oligonucleotide that retains at least some activity of of an oligonucleotide of the disclosure. Such activity is preferably inducing exon skipping of an NF1 pre-mRNA exon disclosed herein, providing a functional NF1 polypeptide or a combination of the foregoing. The activity of a functional equivalent is therefore preferably assessed by detection of exon skipping using the nested PCR read-out disclosed herein and/or quantifying the amount of a functional NF1 polypeptide. A functional NF1 polypeptide is defined as being an NF1 polypeptide able to bind stimulate the GTPas activity of Ras polypeptide, decrease the amount of pERK polypeptide, and or decrease the activity of ELK1 polypeptide using the assays described herein. An assessment of the exon-skipping activity of a functional equivalent is preferably done by the nested PCR read-out described herein. Preferably, the functional equivalent retains at least 50%, at least 60%, at least 70%, at least 80%, at least 90%, at least 95%, at least 98%, or more of corresponding activity of the oligonucleotide of the disclosure from which it was derived. Throughout the application, when the word oligonucleotide is used it may be replaced by a functional equivalent thereof as defined herein.

In one embodiment, distinct oligonucleotides can be combined for efficiently skipping more than one exon in NF1 pre-mRNA. In one embodiment, distinct oligonucleotides can be combined for efficiently skipping a single exon of NF1 pre-mRNA. In one embodiment, a combination of at least two distinct oligonucleotides, a combination of at least three distinct oligonucleotides, a combination of at least four distinct oligonucleotides, or a combination of at least five distinct oligonucleotides are used.

In certain embodiment, an oligonucleotide can be linked to a moiety that enhances uptake of the oligonucleotide by a cell. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

A preferred oligonucleotide comprises a PMO.

The compounds of the invention encompass any pharmaceutically acceptable salts, esters, or salts of such esters, or any other compound which, upon administration to a subject, preferably a human subject, is capable of providing the biologically active metabolite or residue thereof. The disclosure therefore covers prodrugs, pharmaceutically acceptable salts of the compounds disclosed, pharmaceutically acceptable salts of such pro-drugs, functional equivalents and pharmaceutically acceptable salts of such functional equivalents.

In any of the ASOs described herein, the ASO may be substantially uncharged. In any of the ASOs described herein, the ASO is a PMO or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the ASOs described herein, the ASO is interspersed with linkages that are positively charged at physiological pH, where the total number of positively charged linkages is between 1 and no more than half of the total number of linkages.

In certain embodiments, the ASO may have ΔG value between 5 and −1 kcal mol⁻¹, −1 and −11 kcal mol⁻¹, −2 and −10 kcal mol⁻¹, −3, and −9 kcal mol⁻¹, −4 to −8 kcal mol⁻¹, or −5 to −7 kcal mol⁻¹.

A preferred oligonucleotide of the disclosure modulates pre-mRNA splicing in one or more cells of a subject upon systemic delivery. A cell can be provided with a compound of the disclosure capable of interfering with essential sequences, such as but not limited to, an ESE, that result in efficient skipping of an exon of NF1 pre-mRNA by plasmid-derived oligonucleotide expression or viral expression provided by a viral-based vector. Such a viral-based vector comprises an expression cassette that drives expression of an oligonucleotide disclosed herein. Preferred virus-based vectors include adenovirus-based vectors or adeno-associated virus (AAV)-based vectors. Expression is preferably driven by a polymerase III promoter, such as a U1, a U6, or a U7 RNA promoter. Alternatively, a plasmid can be provided by transfection using known transfection agents such as, but not limited to, Lipofectamine™ 2000 (Invitrogen) or polyethyleneimine (PEI; MBI Fermentas), or derivatives thereof.

### Methods of Manufacture

The compounds disclosed herein may be routinely made through the well-known techniques known in the art, including, but not limited to, solid phase synthesis. Any other means for such synthesis of the compounds disclosed herein, particularly ASO, known in the art may additionally or alternatively be employed. One exemplary method for synthesizing compounds disclosed herein is described in U.S. Pat. No. 4,458,066. The techniques of the prior art may be similarly used to manufacture compounds containing 1 or more modified residues, such as, but not limited to, a PMO, PNA, or LNA.

In one embodiment, the compounds disclosed herein are synthesized in vitro and do not include antisense compositions of biological origin. In another embodiment, the compounds disclosed herein are synthesized in vitro, do not include antisense compositions of biological origin, and do not contain genetic vector constructs designed to direct the in vivo synthesis of the compounds. In one embodiment, the compounds disclosed herein are mixed, encapsulated, conjugated or otherwise associated with other molecules or mixtures of compounds, providing liposomes, receptor targeted molecules, oral, rectal, topical or other formulations, for assisting in uptake, distribution, absorption, or a combination of the foregoing of the compounds disclosed herein.

### Pharmaceutical Composition

The present disclosure also describes and provides for pharmaceutical compositions comprising therapeutically effective amounts of a compound described herein, such as an oligonucleotide, including an ASO, together with pharmaceutically acceptable diluents, preservatives, solubilizers, emulsifiers, adjuvants and/or carriers. Suitable additives for a pharmaceutical composition are described in Remington's Pharmaceutical Sciences (Martin, Remington's Pharmaceutical Sciences, 18th Ed. (1990, Mack Publishing Co., Easton, Pa. 18042). Preferably, a compound described herein and included in a pharmaceutical composition described herein is able to induce skipping of an exon of the NF1 pre-mRNA, such as exons 3, 7/8, 9, 10, 11, 12, 14, 17, 18/19, 20, 21, 24, 25, 36, 41, 46, 47, 49, and 52, preferably exons 9, 12, 17, 20, 21, 25, 36, 41, 47, and 52, more preferably exons 9, 12, 17, 25, 41, 47, and 52, and more preferably, exons 17, 47, and 52.

The compounds disclosed herein may be combined with pharmaceutically acceptable diluents, preservatives, solubilizers, emulsifiers, adjuvants and/or carriers to produce a pharmaceutical composition. Suitable carriers and diluents include, for example phosphate-buffered saline. Such compositions include diluents of various buffer types, pH and ionic strength (such as, but not limited to, Tris-HCl, acetate, phosphate, isotonic saline solutions, phosphate buffered saline), and additives such as detergents and solubilizing agents (such as, but not limited to, Tween 80 and Polysorbate 80), anti-oxidants (such as, but not limited to, ascorbic acid, sodium metabisulfite), preservatives (such as, but not limited to, Thimersol, benzyl alcohol) and bulking substances (such as, but not limited to, lactose, mannitol). The compounds described herein may be incorporated into particulate preparations of polymeric compounds such as polylactic acid, polyglycolic acid, hyaluronic acid, or into liposomes. Further excipients include, but are not limited to, Suitable excipients comprise polyethylenimine and derivatives, or similar cationic polymers, including polypropyleneimine or polyethylenimine copolymers (PECs) and derivatives, synthetic amphiphiles, Lipofectin™, DOTAP and/or viral capsid proteins that are capable of self-assembly into particles.

Surfactants such as, but not limited to, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R″′R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

The pharmaceutical compositions may influence the physical state, stability, rate of in vivo release, and rate of in vivo clearance of the compounds disclosed herein. The pharmaceutical compositions may be prepared in liquid form or may be prepared in dry powder form, such as a lyophilised form. The pharmaceutical compositions described herein may be administered by any means known in the art. Preferably, the pharmaceutical compositions are administered parenterally, orally, by the pulmonary, or by the nasal route. In one embodiment, the pharmaceutical compositions described herein are administered by intravenous, intra-arterial, intraperitoneal, intramuscular, or subcutaneous routes of administration. The routes of administration described are intended only as a guide since a skilled practitioner will be able to determine readily the optimum route of administration and any dosage for any particular animal and condition.

The present disclosure also describes the use of the compounds described herein for manufacture of a medicament for modulation of a genetic disease.

In addition, a compound or pharmaceutical composition described herein may contain a targeting ligand specifically designed to facilitate the uptake of the compound or pharmaceutical composition in a cell of interest, cytoplasm and/or its nucleus. Such ligand could comprise (i) a molecule (including but not limited to peptide and peptide-like structures, an antibody, a Fab fragment of an antibody, or a single chain antigen binding domain such as a cameloid single domain antigen-binding domain) recognizing cell, tissue or organ specific elements facilitating cellular uptake and/or (ii) a chemical molecule able to facilitate the uptake of the compound or pharmaceutical composition in a cell and/or the intracellular release of an a compound from a pharmaceutical composition.

The delivery of a therapeutically effective amount of a compound described herein may be achieved by methods previously published. Intracellular delivery of the antisense molecule may be via a composition comprising an admixture of the antisense molecule and an effective amount of a block copolymer (see US Publication No. 20040248833). Other methods of delivery of the compounds described herein may also be used. An expression vector may be used for introducing a nucleic acid sequence coding for a compound described herein into a cell of a subject (see U.S. Pat. No. 6,806,084). The expression vector may be administered as naked DNA, as a part of a viral vector system, or complexed with additional components, such as but not limited to, lipids and/or polymers.

In one embodiment, a compounds described herein is administered as a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. Liposomes are artificial membrane vesicles which are useful as delivery vehicles in vitro, ex vivo, and in vivo methods. These formulations may have net cationic, anionic or neutral charge characteristics. Large unilamellar vesicles (LUV), for example from 100 nm to 500 nm in size, can encapsulate a substantial percentage of an aqueous buffer containing the compounds described herein, allowing the compounds to be encapsulated within the aqueous interior and be delivered to cells in a biologically active form.

In order for an encapsulation approach, such as by liposomes, LUVs and the like, to be an efficient system, one or more of the following characteristics should be present: (1) encapsulation of the compound at high efficiency while not compromising the biological activity of the compound; (2) preferential and substantial binding to a target cell in comparison to non-target cells; (3) delivery of the aqueous contents to the target cell cytoplasm at high efficiency; and (4) accurate and effective expression of genetic information.

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

The pharmaceutical formulations described herein may conveniently be presented in unit dosage form as is known in the art.

Methods and compositions for delivering compounds described herein to a subject are described in: Yang et al., 2020, Mol. Ther. Nucleic Acids, 19, 1357-1367; Juliano, 2016, Nucleic Acids Res., 44, 6518-6548; Roberts et al., Nat Rev Drug Disc., 2020, https://doi.org/10.1038/s41573-020-0075-7; EP Patent No. 2852415; Wang et al., Adv Drug Del Rev, 2015, 87, 68-80; Akhtar et al., 1992, Trends Cell Bio., 2:139; Delivery Strategies for Antisense Oligonucleotide Therapeutics, ed. Akhtar; and PCT WO 94/02595.

In one embodiment, a therapeutically effective amount of a compound described herein, such as an oligonucleotide, including an ASO, is from about 0.1 mg/kg to 50 mg/kg. In another embodiment, a therapeutically effective amount of a compound described herein, such as an oligonucleotide, including an ASO, is from about 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 8 mg/kg, 0.5 mg/kg to 6 mg/kg, 0.5 mg/kg to 4 mg/kg, 0.5 mg/kg to 3 mg/kg, or 0.5 mg/kg to 2 mg/kg. In certain embodiments, the therapeutically effective amount is delivered to a subject according to a course of treatment. As used herein, a “dose” refers to an effective amount of a compound disclosed herein delivered at a given time point, such as a time point specified in a course of treatment.

In certain embodiments, more than one dose of a compound of the disclosure is administered during a course of treatment. Therefore, in the methods described herein, the methods may comprise the administration of multiple doses during the course of treatment. In certain embodiments, the course of treatment may range from 1 month to years. In certain embodiments, the course of treatment is continuous throughout the life of the subject. In certain embodiments, a dose is administered at least 1 time per week during the course of treatment. In certain embodiments, a dose is administered at least 1 time every other week during the course of treatment. In certain embodiments, a dose is administered at least 1 time every three week during the course of treatment. In certain embodiments, a dose is administered at least 1 time every month during the course of treatment. Furthermore, the amount of a compound of the disclosure in each dose need not be the same as discussed above.

In one embodiment, a course of treatment may comprise administering at least one dose as a loading dose and at least one dose as a maintenance dose, wherein the loading dose contains a greater amount of a compound of the disclosure as compared to the maintenance dose (such as, but not limited to, 2 to 10 times higher). In one aspect of this embodiment, the loading dose is administered initially, followed by administration of one or more maintenance doses through the remaining course of treatment. For example, for a course of treatment that is one time per week for the life of the subject, a loading dose of 50 mg/kg may be administered as the first dose on week 1 of the course of treatment, followed by maintenance doses of 25 mg/kg for the remainder of the course of treatment. Furthermore, a loading dose may be given as a dose that is not the first dose administered during a course of treatment. For example, a loading dose may be administered as the first dose on day 1 and as a dose one additional day (for example, day 4). For example, for a course of treatment that is one time per week for the life of the subject, a loading dose of 50 mg/kg may be administered as the first dose on week 1 of the course of treatment, followed by maintenance doses of 25 mg/kg for at weeks 2 to 25, followed by a second loading dose of 40 mg/kg on week 26, followed by maintenance doses of 25 mg/kg for the remainder of the course of treatment. When more than one loading dose is administered during a course of treatment, the loading dose may be the same (i.e., 10 mg/kg) or different (i.e., 20 mg/kg for the first loading dose and 10 mg/kg for each other loading dose).

In a first embodiment, the present disclosure provides a composition comprising an antisense oligonucleotide comprising a sequence that is specifically hybridisable to a target sequence in a pre-mRNA in a NF-1 exon.

In one aspect of the first embodiment, the antisense oligonucleotide is identified by the methods described herein. In another aspect of this embodiment, the antisense oligonucleotide contains 20-60 subunits, preferably 20-35 subunits. In another aspect of the first embodiment, the antisense oligonucleotide is specifically hybridisable with one of exons 3, 7/8, 9, 10, 11, 12, 13, 14, 17, 18/19, 20, 21, 24, 25, 36, 41, 46, 47, 49, and 52, preferably exons 9, 12, 13, 17, 20, 21, 25, 36, 41, 47, and 52, more preferably exons 9, 12, 13, 17, 25, 41, 47, and 52, and more preferably, exons 17, 47, and 52. In another aspect of the first embodiment, the antisense oligonucleotide comprises a sequence selected from the group consisting of SEQ ID NOS: 1-24, 49, 51, or 53.

In a second embodiment, the present disclosure provides a composition, comprising an antisense oligonucleotide comprising a sequence that is specifically hybridisable to a target sequence in exon 17 of NF1 pre-mRNA. In another aspect of this embodiment, the antisense oligonucleotide contains 20-60 subunits, preferably 20-35 subunits. In one aspect of this embodiment, the antisense oligonucleotide is selected from SEQ ID NOS: 1-6 or 49. A suitable target sequence from exon 17 includes, but is not limited to, SEQ ID NOS: 25-30 or 50 or a continuous stretch of at least 20 nucleotides within SEQ ID NO: 57. Preferably, the target sequence comprises an ESE.

In a third embodiment, the present disclosure provides a composition, comprising an antisense oligonucleotide comprising a sequence that is specifically hybridisable to a target sequence in exon 47 of NF1 pre-mRNA. In another aspect of this embodiment, the antisense is oligonucleotide contains 20-60 subunits, preferably 20-35 subunits. In one aspect of this embodiment, the antisense oligonucleotide is selected from SEQ ID NOS: 7-12 or 51. A suitable target sequence from exon 47 includes, but is not limited to, SEQ ID NOS: 31-36 or 52 or a continuous stretch of at least 20 nucleotides within SEQ ID NO: 63. Preferably, the target sequence comprises an ESE.

In a fourth embodiment, the present disclosure provides a composition, comprising an antisense oligonucleotide comprising a sequence that is specifically hybridisable to a target sequence in exon 52 of NF1 pre-mRNA. In another aspect of this embodiment, the antisense oligonucleotide contains 20-60 subunits, preferably 20-35 subunits. In one aspect of this embodiment, the antisense oligonucleotide is selected from SEQ ID NOS: 13-18 or 53. A suitable target sequence from exon 52 includes, but is not limited to, SEQ ID NOS: 37-42 or 54 or a continuous stretch of at least 20 nucleotides within SEQ ID NO: 64. Preferably, the target sequence comprises an ESE.

In a fifth embodiment, the present disclosure provides a composition, comprising an antisense oligonucleotide comprising a sequence that is specifically hybridisable to a target sequence in exon 13 of NF1 pre-mRNA. In another aspect of this embodiment, the antisense oligonucleotide contains 20-60 subunits, preferably 20-35 subunits. In one aspect of this embodiment, the antisense oligonucleotide is selected from SEQ ID NOS: 19-24. A suitable target sequence from exon 13 includes, but is not limited to, SEQ ID NOS: 43-48 or a continuous stretch of at least 20 nucleotides within SEQ ID NO: 65. Preferably, the target sequence comprises an cryptic splice site.

Such a composition of the first to fourth embodiments may be used in the methods of treatment and methods of exon skipping as described herein.

Such a composition of the fifth embodiment may be used in the methods of treatment and methods of exon retention as described herein.

In any of the compositions described herein, particularly the first to fifth embodiments above, the ASO is substantially uncharged. In any of the compositions described herein, particularly the first to fifth embodiments above, the ASO is a PMO or contains one or more morpholino subunits. In certain aspects, the morpholino subunits are linked by phosphorus-containing inter-subunit linkages joining a morpholino nitrogen of one subunit to a 5′ exocyclic carbon of an adjacent subunit. In any of the compositions described herein, particularly the first to fifth embodiments above, the ASO is interspersed with linkages that are positively charged at physiological pH, where the total number of positively charged linkages is between 1 and no more than half of the total number of linkages.

In any of the compositions described herein, particularly the first to fifth embodiments above, the ASO is identified by the methods described herein.

In any of the compositions described herein, particularly the first to fifth embodiments above, the ASO may have ΔG value between 5 and −1 kcal mol⁻¹, −1 and −11 kcal mol⁻¹, −2 and −10 kcal mol⁻¹, −3, and −9 kcal mol⁻¹, −4 to −8 kcal mol⁻¹, or −5 to −7 kcal mol⁻¹.

### Kits

The disclosure also describes and provides for kits for treatment of a patient with a genetic disease, preferably NF1, the kit comprising at least compound described herein, preferably an oligonucleotide, including an ASO, packaged in a suitable container, together with instructions for use (such as, but not limited to, administration to a subject).

In one embodiment, the kit will contain at least one compound described herein selected from the group consisting of SEQ ID NOS: 1-18, 49, 51, or 53. The kit may also contain accessory reagents such as buffers, stabilizers, and the like as described herein.

### EXAMPLES

### Example 1—in Silico Analysis and Prioritization of Exons

FIG. 1 represents NF1 exons. Initial evaluation of the NF1 transcript identified 25 single and an additional 18 exons representing consecutive exons pairs that could be skipped while maintaining the translational reading frame (covering 43 of 58 exons). This represents a significant portion (74%) of the transcript that is potentially available for exon skipping therapeutics. FIG. 1 also denotes some of the various domains (Scheffzek, et al., (2012). In Neurofibromatosis Type 1: Molecular and Cellular Biology, M. Upadhyaya and D. N. Cooper, eds. (New York, Springer), pp 305-325). The best characterized is the GAP-related domain (GRD) encoded by exons 27-35.

Subsequently, the literature and publicly available datasets (LOVD and HGMD) were mined for reports of NF1 patients with identified mutations that demonstrably produced exon skipping or deleted exons in the mature mRNA. The results indicate that 49 out of the 58 individual exons, can be found deleted or skipped in patient transcripts. From those that can be deleted while maintaining the reading frame, only four exons did not appear in the search, namely exons 17, 25, 31, and 52. As exon 31 also lacks pathogenic mutations (none have been reported in databases), this exon was excluded from further analysis (denoted by black boxes in FIG. 1) as it would not be a therapeutic target for exon skipping. The remaining exons were prioritized for in vitro analysis. Further, of the consecutive exon pairs (6+7, 7+8, 15+16. 18+19, 29+30, 37+38, 42+43, 44+45, 50+51, 56+57, 57+58) only 6/7 skipping/deletion was reported in an NF1 patient (documented in the UAB Medical Genomic Laboratory but not in public databases). The findings for single exons are summarized in FIG. 1 (row “Patients”).

Next exon length was evaluated as, intuitively, longer exons might be proportionally at higher risk for being more relevant to function (see FIG. 1, row “Length (nts)”). For instance, the longest exon is exon 21 with 441 nucleotides and was found deleted in NF1 patients, suggesting exon 21 is essential. Additional long exons had already been discarded due to their skipping introducing a frameshift or known pathogenicity from reported NF1 patients. However, prioritized exons 17, 25, and 52 which have no patients reported with skips have similar lengths between 117 and 156 nucleotides, where 135 is the median exon length.

Since protein function is often associated with protein PTMs, information was gathered about the exon localization of experimentally verified PTMs, in particular phosphorylation, ubiquitination, and acetylation. Given phosphorylation is the most common PTM, phosphorylation was considered as likely more important for NF1 function than other PTMs. Consequently, exons containing residues that have been experimentally verified to be phosphorylated are highlighted (FIG. 1, row “PTMs”, marked dark red), while numbers refer to the number of modified residues in the respective exon. With few exceptions, most phosphorylation data were obtained through high throughput proteomic mass spectrometry, and neither their function nor the responsible kinase are known. While there are no known PTMs of residues in exon 25, exon 17 carries one potential phosphorylation site. Six phosphorylation sites have been reported for exon 52, including at positions T2554 by PKA (Feng, L. et al., (2004), FEBS Lett 557, 275-282) and Y2556 (Jorgensen, C. et al., (2009), Science 326, 1502-1509), with known functional roles. The fact that no missense mutations of the T2554 and Y2556 residues have yet been reported argues for their importance.

PredictProtein results for neurofibromin complemented the first evaluation of exons for the purpose of therapeutic exon skipping. FIG. 1 summarizes the results of predicted features including solvent accessibility, quantified in terms of number of residues predicted to be exposed (row “Accessibility”), disorder status in terms of number of residues predicted to be exposed (row “Disorder”), percentage of Non-ORdinary secondary structure contributed by each exon (row “NORS”), the average conservation score over all residues associated with an exon (row “Average Conservation”) as well as the number of maximally conserved residues (row “Maximum Conservation”). With respect to solvent accessibility, the four prioritized exon candidates (exons 17, 25, and 52) are very similar (between 9 and 13 residues are classified as exposed), while individual exon's contributions to neurofibromin's surface can be significantly higher (e.g. exon 21 is associated with 46 residues predicted to be exposed). While is most exons are fully ordered (including exons 17 and 25), a few have residues classified as disordered. Among those exons 13, 21, 50, 52, 57, and 58 generate more than 19 such residues, while the only two long (>=30 residues) intrinsically disordered regions (IDRs) are produced by exons 51-52 and 57-58. Only exons 50-53 and 57-58 contribute to a predicted non-ordinary secondary structure. Of note, IDRs allow a protein to adopt an ensemble of different conformations, which are thought to be in dynamic equilibrium under physiological conditions (Babu, M. M. (2016), Biochem Soc Trans 44, 1185-1200). Long IDRs, such as the one produced by exons 51 and 52, are also known to increase the degradation efficiency by the proteasome, and consequently regulating protein half-life (van der Lee, R., Lang, B., et al., (2014). Cell Rep 8, 1832-1844), which could potentially be beneficial in the context of therapeutic exon skipping. On the other hand, IDRs are known to contribute to a protein's functionality in various ways, including but not limited to providing a multitude of binding modes to other proteins, often allowing PTMs to modify the binding kinetics (Uversky, V. N. (2019), Frontiers in Physics 7). Lastly, the obtained conservation scores strongly suggest that exon 25 is likely not suitable target for exon skipping with high score of 8.1. In contrast, exons 17 and 52 have low conservation scores of 1.2 and 2.2 respectively, signifying potential suitability as a target for exon skipping.

Following this first round of analysis of NF1 as a full-length protein, exons were selected to model what might happen if they were deleted. All exons that, when deleted/skipped, produce a frameshift, and consequently a truncated protein, were discarded. Likewise, initially all exons reported as deleted in the mature transcript in at least one NF1 patient were deprioritized. This reduced the number of potential candidates for single exon skipping-based therapy to three: exon 17, 25, and 52. For additional in-depth in silico analysis, an additional eight single exon were chosen, namely exon 9, 1, 20, 21, 28, 36, 41 and 47, all of which retain frame when being skipped but are found in patients with an NF1 phenotype. The selection of the additional eight exon skipping scenarios was partially based on the lack of information of pathogenicity from NF1 patients that had been reported with such exons deleted/skipped in NF transcripts, as well as to provide control exon that, when skipped, are known to produce non-functional proteins (such as exon 28 that codes for part of the GRD and the critical R1276 “arginine finger” amino acid that binds Ras-GTP; Scheffzek, K., et al., (2012), In Neurofibromatosis Type 1: Molecular and Cellular Biology, M. Upadhyaya and D. N. Cooper, eds. (New York, Springer), pp 305-325). Moreover, from the set of consecutive exon pairs, exon 18/19 were included in the analysis. By further studying these protein, additional information about the impact that individual exon skipping likely has on retaining neurofibromin functionality was obtained.

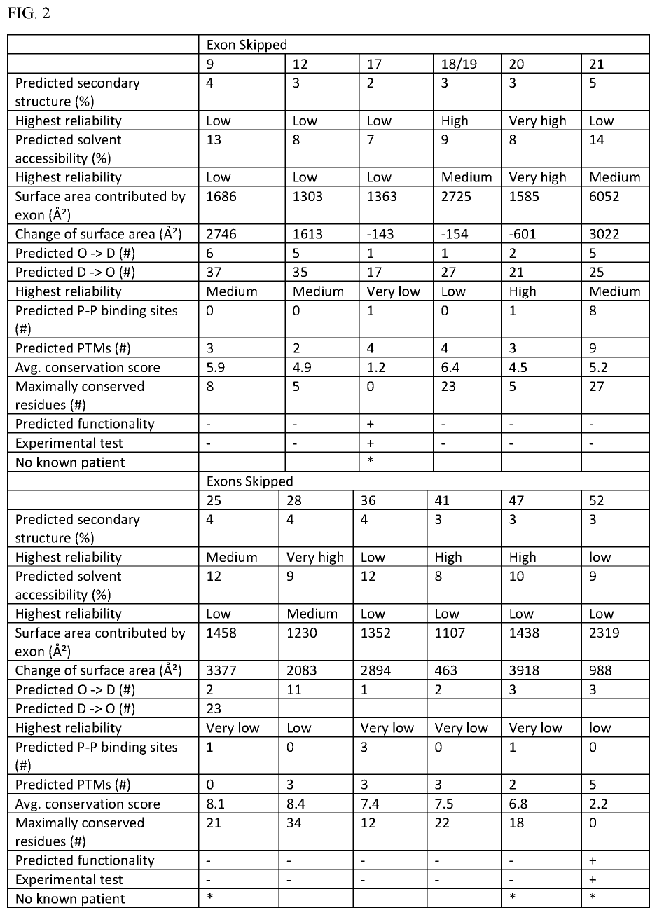

The assessment of the likelihood that skipping an individual exon or exon pair has a therapeutic effect is based on various factors. This data is summarized in FIG. 2 and detailed in Table 1 below.

TABLE 1

| Exon | Details | Conclusion |
|---|---|---|
| 9 | Predictions suggest that deleting exon 9 impacts the secondary structure of | Deletion |
| | at most 4% and the solvent accessibility of at most 13% of remaining | could |
| | residues, if any, as reliability for these predictions are very low or low. | potentially |
| | Solvent accessibility of NF1delE9 is likely reduced beyond what can be | reduce the |
| | attributed to the deleted exon. 11 out of 13 residues at positions 801-813 are | protein's |
| | predicted to be ordered (from disordered in full-length NF1). This suggests | flexibility |
| | that the exon deletion could potentially reduce the protein's flexibility in this | and function |
| | area, possibly affecting protein function. However, with the exception of | |
| | one such prediction that has medium reliability, predictions have very low | |
| | to low reliability. Average Conservation score for this exon is rather | |
| | moderate at 5.9. | |
| 12 | Predictions suggest that deleting Exon 12 impacts the secondary structure of | Deletion |
| | at most 3% and the solvent accessibility of at most 8% of remaining | could |
| | residues, if any, as reliability for these predictions are very low or low. | potentially |
| | Solvent accessibility of NF1delE12 may be somewhat reduced beyond what | reduce the |
| | can be attributed to the deleted exon. A 19 residue long sequence, starting | protein's |
| | with the residue produced by the first codon after the deleted exon, is now | flexibility, |
| | predicted to be ordered (from disordered in full-length NF1) - some of these | possibly |
| | individual predictions have medium reliability. This suggests that the exon | affecting |
| | deletion could potentially reduce the protein's flexibility in this area, | protein |
| | possibly affecting protein function. Average Conservation score for this | function. |
| | exon is rather moderate at 4.9. | |
| 17 | Predictions suggest that deleting Exon 17 impacts the secondary structure | This |
| | by at most 2% and the solvent accessibility of at most 7% of remaining | suggests that |
| | residues, if any, as reliability for these predictions are very low or low. | the deletion |
| | NF1delE17's solvent accessibility seems somewhat similar to that of | of exon 17 |
| | neurofibromin minus the solvent accessibility attributed to amino acids | seems to |
| | translated from exon 17. Differences in predicted residue order/disorder | affect the |
| | state are minimal, and all have the lowest reliability score. Average | protein's |
| | Conservation score for this exon is the lowest of all exons at 1.2. | function very |
| | | little, if at |
| | | all. |
| 18/19 | Predictions suggest that deleting Exons 18/19 impacts the secondary | Overall, it |
| | structure of at most 3% of residues. While most predictions have very low | appears that |
| | or low reliability, four predicted changes have medium (4 or 5) and one has | the CSRD |
| | high (6) reliability, out of which those with reliability 5 or higher are found | could be |
| | in the CSRD, suggesting that deleting Exons 18/19 affects the structure of | affected by |
| | the CSRD. It is further predicted that the solvent accessibility of at most 9% | E18/19 |
| | of remaining residues changes. Again, the reliabilities of most of these | deletion. |
| | predictions are very low or low, with the exception of one predicted residue, | Deletion is |
| | which changed from intermediate to buried and is located in the CSRD. | predicted to |
| | That said, the average solvent accessibility change per residue is roughly | alter |
| | zero. Out of 27 residues predicted to change from ordered to disordered, 17 | secondary |
| | are in a 20-residue long sequence within the CSRD (pos. 796-815 in NF1fl), | structure |
| | but prediction reliabilities are all very low or low. Average Conservation | |
| | scores for both exons are rather moderate at 6.4. | |
| 20 | Only 3% of residues are predicted to change their secondary structure. | In |
| | While the majority of predictions has very low or low reliability, 11 | conclusion, |
| | predicted changes of residue secondary structure have high or very high | the protein |
| | reliability. All these residues are located in a 14 residue long subsequence | structure and |
| | (pos. 804-817) of the CSRD. The same sequence has predicted changes to | conformation |
| | solvent accessibility and order/disorder status with medium to very high and | could be |
| | medium to high reliability, respectively (in total, at most 8% of residues are | significantly |
| | predicted to have an altered solvent accessibility status). Interestingly, the | altered by |
| | change of average solvent accessibility per residue is negative, when | the deletion |
| | compared to neurofibromin, implying an increase of solvent accessibility for | of exon 20. |
| | the remaining residues in NF1del20. Average Conservation score for this | |
| | exon is rather moderate at 4.5. | |
| 21 | 5% of residues have a different predicted secondary structure, albeit with | This |
| | low or very low reliability, while for 14% a changed solvent accessibility is | indicates a |
| | predicted, again with low or very low reliability - with one exception: a | potential |
| | residue in the CSRD, which is adjacent to the deleted exon sequence. | increase in |
| | NF1delE21 has likely a reduced overall solvent accessibility, beyond what | flexibility in |
| | is attributed to the loss of exon 21. A cluster of eight consecutive residues | this region of |
| | located in the CSRD at positions 796-803, adjacent to the deleted exon in | the protein, |
| | NF1fl, is predicted to be disordered (compared to ordered in full-length | possibly |
| | NF1) with medium reliability. Average Conservation score for this exon is | affecting |
| | rather moderate at 5.2. | function. |
| 25 | 4% of NF1delE25's residues have a different predicted secondary structure, | Conservation |
| | mostly with low or very low reliability (for 3 residues, predicted changes | of this exon |
| | have medium reliability). For 12% of residues a changed solvent | is quite high |
| | accessibility is predicted. The protein's solvent accessibility seems reduced | with a score |
| | as indicated by the relative high loss of average solvent accessibility per | of 8.1 |
| | residue, the second highest of all tested proteins with deleted exons. Also, | suggesting |
| | there is a cluster of 11 residues at position 801-813 (in full-length NF1) that | this exon |
| | is predicted to be ordered, indicating a loss of flexibility. However, | might be |
| | prediction reliabilities are very low, which limits the interpretability of these | essential. |
| | results. Conservation of this exon is quite high with a score of 8.1 | |
| | suggesting this exon might be essential. | |
| 28 | NF1 structure is likely significantly affected by exon 28 deletion: 7 | We fully |
| | predicted changes have medium, 3 have high, and another 8 have very high | anticipate |
| | reliability; and 7 out of those 8 are related to residues in the GRD, while the | that loss of |
| | 8th is for a residue within the Nex-GRDmin-Ces region. We hypothesize | this exon |
| | that this has a direct negative impact on Ras/Spred1 binding. Moreover, 5 | will result in |
| | residues are predicted, with medium reliability, to have changed solvent | loss of GRD |
| | accessibility status, out of which 3 are found in the GRD, one is right | activity as |
| | outside the GRD and another is in the Tubulin binding domain. | this exon |
| | NF1delE28's overall solvent accessibility seems slightly reduced, but | codes for a |
| | residues in the GRD seem to be affected the most. Conservation of this | known |
| | exon is high with a score of 8.4 suggesting this exon might be essential. | essential |
| | | portion of |
| | | the GRD. |
| 36 | All predictions, i.e. changes to secondary structure (4% of residues), solvent | Conservation |
| | accessibility (12% of residues), disorder/order status, and protein binding | of this exon |
| | have low or very low reliability, limiting the interpretability of the data. | is quite high |
| | That said, NF1delE36 has an overall solvent accessibility that is reduced | with a score |
| | beyond what can be attributed to the loss of exon 36. Also, 11 resides in the | of 7.4 |
| | CSRD (pos. 801-813) may have changed from disordered to ordered, which | suggesting |
| | would potentially reduce the protein's flexibility. Conservation of this exon | this exon |
| | is quite high with a score of 7.4 suggesting this exon might be essential. | might be |
| | | essential. |
| 41 | Only 3% of residues are predicted to have changed their secondary | Overall, |
| | structure. While the majority of predictions have very low or low reliability, | structural |
| | 3 predicted changes of residue secondary structure have medium or high | changes are |
| | reliability (all outside known domains). Also, 8% of residues have been | likely and |
| | predicted to change their solvent accessibility state, but all predictions have | conservation |
| | low or very low reliability. Total solvent accessibility of NF1delE41 seems | of this exon |
| | similar to that of neurofibromin, if we disregard the lost solvent accessibility | is high with |
| | due to the deleted exon. Predictions of changes to the order/disorder status | a score of 7.5 |
| | of residues have low or very low reliability and only occur in isolated single | suggesting |
| | or pairs or residues. Conservation of this exon is high with a score of 7.5 | this exon |
| | suggesting this exon might be essential. | might be |
| | | essential. |
| 47 | 3% of residues are predicted to have changed their secondary structure. | Overall, |
| | While most predictions have low or very low reliability, 6 predictions have | structural |
| | medium and 3 predictions have high reliability (but all these residues are | changes are |
| | outside any known domain). 10% of residues are predicted to have changed | indicated and |
| | their solvent accessibility state. All predictions except one have low or very | could in |
| | low reliability. The average loss of solvent accessibility per residue is the | principle |
| | highest of all tested proteins with deleted exon(s). 19 residues within a 30 | impact |
| | residue long sequence are predicted to have a change from disordered to | proper |
| | ordered, albeit with low reliability. Conservation of this exon is moderate | function. |
| | with a score of 6.8 | |
| 52 | Only 3% of residues are predicted to have changed their secondary | Overall, |
| | structure. All predictions has very low or low reliability. For 12% of | structural |
| | residues a changed solvent accessibility is predicted, but reliabilities are low | changes do |
| | or very low. There is only a small additional loss of solvent accessibility (on | not appear to |
| | top of the loss due to exon 52 deletion). Predictions of changes to the | be dramatic, |
| | order/disorder status of residues have low or very low reliability and mostly | but PTMs |
| | occur in isolated single or pairs or residues. Conservation of this exon is low | and NLS will |
| | with a score of 2.2. Overall, structural changes do not appear to be | be lost. |
| | dramatic. Of concern is the loss of phosphorylation sites as mentioned in | |
| | the main text: Exon 52 is phosphorylated at T2554 by PKA, which was | |
| | shown to regulate interaction with 14-3-3 beta (human) and at Y2556. Loss | |
| | of NLS is also a concern. | |

Exon 17 appears to be most promising as a therapeutic target for exon skipping as changes to secondary structure, solvent accessibility, order, protein binding sites and PTMs would be minimal. Effects of exon 17 loss on the function of the CSRD are unknown. Exon 52 may also be a good candidate due to minimal predicted changes in secondary structure, solvent accessibility, order, and protein binding sites; however, the effects of the loss of the PTMs and its encoded nuclear localization signal (NLS) (Vandenbroucke I, et al., (2004), FEBS Lett., 560(1-3):98-102) are unknown. All other exons have relatively high average conservation scores (in particular exons 25, 28, 36, and 41) and/or a large number of maximally conserved residues (such as 18/19, 21, 25, and 47) indicating crucial functionality. Further, the secondary and tertiary structure of the protein may change dramatically when skipping exons 18/19, 20, 28, 41, and 47. Deletion of exon 21 would result in loss of PTMs and predicted protein binding sites. Finally, the skipping of exons 9, 12, 20 and 21 may result in proteins with less flexibility and hence some loss of functionality.

### Example 2—Testing in cDNA Assay System

In efforts to both evaluate the in silico predictions and determine the functional effects of exon skipping on neurofibromin, Nf1 cDNAs with deletions of specified exons: 9, 12, 17, 20, 21, 25, 28, 36, 41, 47, and 52 (indicated by bolded boxes in FIG. 1) were created and tested. Deletion of both exons 18 and 19 consecutively was also evaluated. Synthetic gene fragments were used to create these deletions and cloned them into our full-length mNf1 cDNA clone. All clones were validated by full length sequencing of each plasmid. All cDNAs representing the various exon skips were evaluated in four different functional assays.

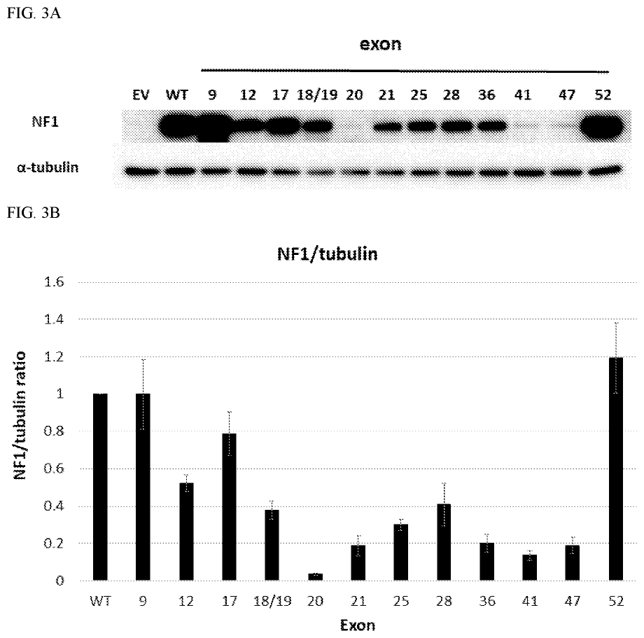

First, the level of NF1 protein expressed in NF1 null HEK293 cells when transiently transfected with a constant amount of cDNA was determined. Cells were seeded at 500,000 cells per 6-well and transfected with 1 μg of each individual cDNA. A representative Western blot probed with NF1 antibody is shown in FIG. 3A (also showing tubulin as a loading control). A minimum of 3 separate experiments were conducted and quantitated, with the results depicted in FIG. 3B as NF1/tubulin ratio. All data is shown normalized to the WT cDNA. Loss of some exons leads to decreased NF1 levels presumably due to loss of protein stability and/or increased protein degradation. This is especially apparent for deletion of exons 20, 21, 41 and 47 which show the lowest levels of NF1 protein. Loss of other exons such as 9 and 17 seems to have little effect on NF1 protein levels. Loss of exon 52 leads to increased NF1 protein levels. Based on the in silico analysis with UbiProber which computationally predicts eukaryotic ubiquitylation sites, exon 52 encodes a polyubiquitination site. NF1 is known to be regulated by proteolysis and Cul3. It is possible that loss of this site leads to less ubiquitination and less targeting of the protein to the proteasome for degradation. Furthermore, deletion of exon 52 leads to loss of IDRs. Long IDRs, such as the one produced by exons 51 and 52, are also known to increase the degradation efficiency by the proteasome, and consequently regulating protein half-life. As disordered regions target proteins for degradation, this loss may diminish that targeting and result in higher levels of protein.

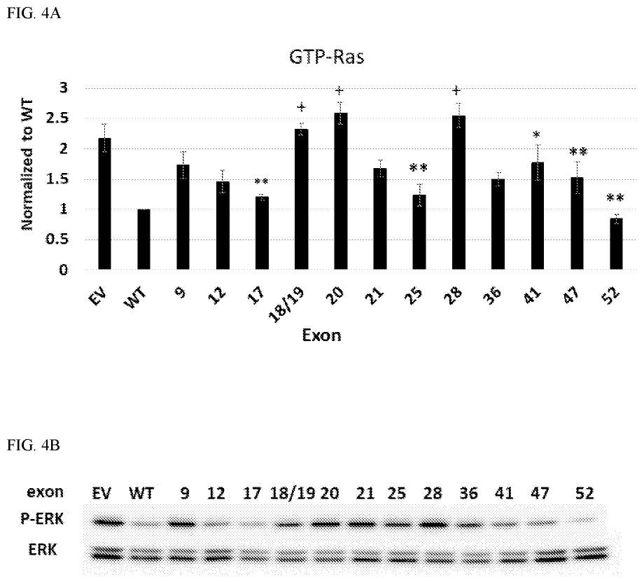

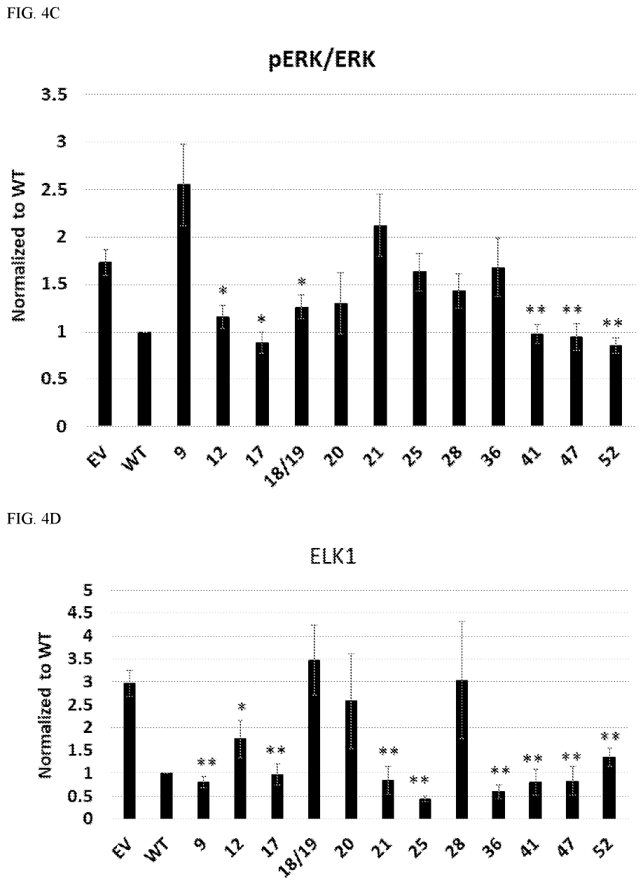

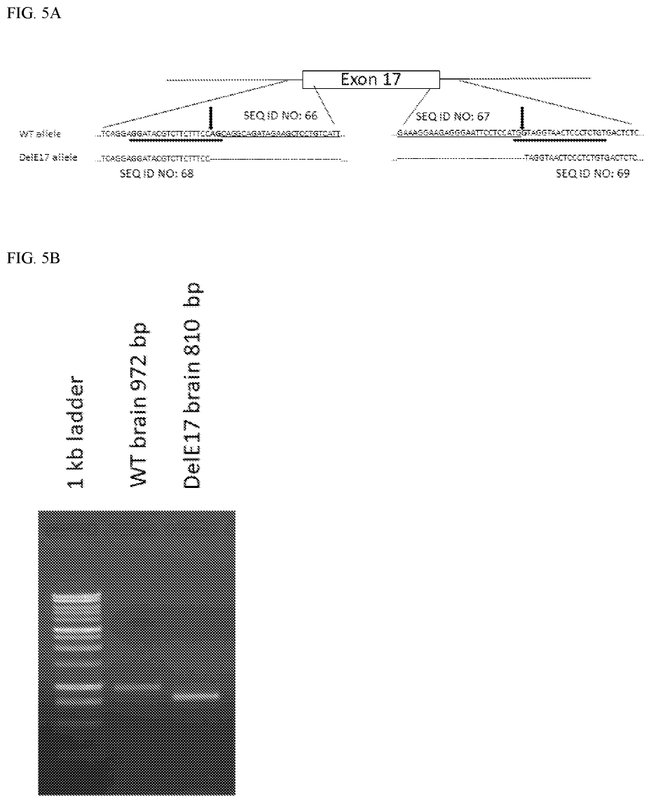

Second, the levels of GTP-Ras (FIG. 4A) were evaluated. All samples were normalized to WT control and evaluated in at least 3 experiments. GTP-Ras levels of all mutant cDNAs were statistically compared to that of EV cDNA by t-test. Exons that were significantly better (p<0.05) than empty vector cDNA (i.e., those that retain at least some ability to suppress levels of GTP-Ras) include: 17, 25, 41, 47, and 52. Conversely, those exons that are statistically worse than EV (i.e., those that do not retain ability to suppress levels of GTP-Ras) include exons 18/19, 20, and 28.

Third, p-ERK/ERK ratios were evaluated (FIG. 4B and C). All samples were normalized to WT cDNA and evaluated in at least 3 experiments. p-ERK/ERK levels of all mutant cDNAs were compared to that of EV cDNA by t-test. Exons that were significantly better than EV cDNA (i.e., those that retain ability to suppress levels of p-ERK) include: 12, 17, 18/19, 20, 41, 47, and 52.

Fourth, ELK-1 luciferase activity was evaluated (FIG. 4D). All samples were normalized to WT cDNA control and evaluated in at least 3 experiments. Luciferase levels of all mutant cDNAs were compared to that of EV cDNA by t-test. Exons that were significantly better than EV cDNA (i.e., those that retain ability to suppress levels of ELK-1) include: 9, 12, 17, 21, 25, 36, 41, 47, and 52.

In general, the in silico data strongly predicts the results of in vitro assays. For example, those exons that were predicted to have the least effects by PredictProtien (exons 17 and 52) have the highest levels of neurofibromin and ability to suppress Ras activity in vitro. The exons with the highest neurofibromin levels (9, 17, and 52) had the lowest percent of residues predicted to undergo changes in secondary structure when compared to full-length neurofibromin. Conversely, exons 20, 41, and 47 were predicted to significantly alter secondary structure and deletion of these exons lead to the lowest levels of neurofibromin expression in the in vitro assay. Those that performed worst in the GTP-Ras assay (18/19, 20, and 28) were all predicted to have significant changes in secondary structure. Notably, exon 28 deletion performed the worst in Ras assays as it retained the least function. This is likely because it encodes a portion of the GRD domain that physically interacts with Ras including the “arginine finger” residue R1276. These results show the in silico model is predictive.

While it is true that the individual cDNAs performed differentially between Ras assays in terms of whether or not they were statistically similar or different than EV cDNA, the same trends are observed across the 3 functional assays for each individual cDNA. It is also important to note that these assays tend to have relatively small dynamic ranges and a correlation between the target assayed, its location within the Ras pathway, and the dynamic range observed of the WT and EV cDNAs has been previously reported (Wallis, D., et al., (2018), Hum Mutat 39, 816-821). For example, WT cDNA is best able to repress ELK1 transcription, the target most distal from Ras. Similarly, WT cDNA shows intermediate levels of p-ERK repression and mild repression of GTP-bound Ras levels. This most likely corresponds to signal amplification of targets more distal to Ras. Hence, more of the test exons show significance as the assay moves distally from Ras. For example, five exons show significant changes in GTP-Ras levels, six show significant changes in pERK/ERK ratios, and nine show significant changes in ELK1 transcriptional activity.

It has been reported previously (Wallis, D., et al., (2018), Hum Mutat 39, 816-821) that some cDNAs may lead to hyperactivation of GTP-Ras levels above what is seen with EV (without NF1). Notably, deletion of exons 18/19, 20, and 28 lead to increased or exacerbated GTP-Ras levels (FIG. 4A). In the previous study a similar response was observed with a cDNA carrying the R681X mutation which maps within exon 18. This mutation also leads to a mouse with an exacerbated phenotype over a simple deletion of Nf1. Such data may imply that these changes could impede hydrolysis of Ras-GTP. While no such evidence was observed in the present analysis of pERK/ERK levels for these exons (FIG. 4B and C), it is interesting to note that exons 18/19, 20, and 28 are the only clones that were not able to suppress ELK1 transcriptional activity (FIG. 4D). Hence, it is likely that these exons contain regions of NF1 that are essential to its GRD function. While this is explicitly known for exon 28 the significance of exons 18/19 and 20 was previously unclear. These exons code for amino acids 670-805 within the CSRD domain and are just upstream (but not overlapping) amino acids 844-848 which have a known genotype-phenotype correlation associated with a more severe NF1 phenotypes (Koczkowska, M., et al., (2018), Am J Hum Genet 102, 69-87). Hence, this region (exons 18-21) may be critical and likely should not be targeted for exon skipping. These data may also result from the fact that neurofibromin has recently been shown to dimerize/oligomerize (Mellert, K., et al., (2018), Sci Rep 8, 6171; Carnes, R. M., et al., (2019), Genes 10(9), 650-667). While regions critical for dimerization have not yet been reported, it is possible that they overlap with these critical exons.

Even though some cDNAs result in lower levels of neurofibromin expression (presumably due to loss of stability) this doesn't equate to loss of GRD function. In fact, some of the cDNAs with lower amounts of NF1 protein have the most robust ability to inhibit Ras activity. This has been reported for exclusion of the alternatively spliced exon 31 (Andersen L B, et al., (1993), Mol Cell Biol., 13(1):487-95; Yunoue S, et al., (2003), J Biol Chem., 278(29):26958-69). If these cDNAs indeed lose stability, but retain function, treatment with small molecules to stabilize NF1, such as protein correctors, including tezacaftor or lumacaftor used to stabilize the CFTR protein in cystic fibrosis, might be a promising therapy.

The initial in silico analysis included all constitutively expressed exons. The in vitro analysis included all exons without reported exon skips in NF1 patients and also expanded to include additional exons that maintained frame but had some patients reported. The following exons were not tested in vitro even though they would maintain frame: 2, 3, 10, 11, 13, 14, 23, 24, 32, 34, 35, 46, and 49. The following exon pairs were not tested even though skips of these exons have not been reported: 7/8, 15/16, 29/30, 37/38, 42/43, 44/45, 50/51, or 56/57. Of these, exons 29/30, 32, 34, and 35 encode the GRD and are likely essential code for the GRD and are likely essential. The secondary analysis indicates that exons 2, 13, 42/43, and 50/51 code for PTMs, and that exons 15/16, 23, 37/38, 42/43, 44/45, and 56/57 are conserved with individual exon conservation score greater than 7, arguing their likely essentiality. This leaves exons 3, 7/8, 10, 11, 14, 24, 46, and 49 unevaluated in vitro, despite possible residual function. All have no or questionable PTMs, low accessibility, low disorder, no non-ordinary secondary structure, and moderate conservation. While the report of patients with these in-frame exon skips makes them less suitable as exon skipping targets, if the resultant exon skip primarily results in lowered neurofibromin levels with functional GRD activity, then this issue might be resolved with combination therapy of ASOs and corrector compounds. This data can lead to future structure-function studies and possibly help lead to a “mini-NF1” gene cassette for gene replacement therapies utilizing viral vectors.

The data in the present disclosure and cDNA system is the first demonstration that distinct differences in NF1 levels (stability) and GRD function, including increased GTP-Ras levels for various cDNAs, can be determined. This is significant as genotype phenotype correlations are beginning to be made and it is possible that mutations that result in loss of NF1 levels might have different phenotypic correlations or treatment modalities available in contrast to mutations that lead to loss of GRD function. Collectively the data suggest that exon skipping in NF1 can result in at least partial GRD function. Skipping of exons 17, 40, 46, and 51 appears most promising with exons 17 and 51 having a clear advantage to 40 and 46 due to higher levels of neurofibromin, though additional exons may be added to this list (such as, but not limited to, exons 3, 10, 11, 14, 24, 45, and 48).

### Example 3—Mouse Model DelE17

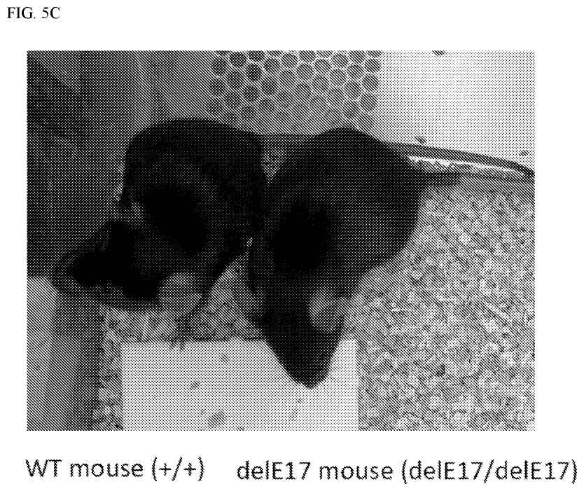

To test the essentiality of exon 17, a mouse was created that carries Nf1 alleles with exon 17 completely deleted (DelE17) (FIG. 5A). This allele removes −2 basepairs 5′ of exon 17 and +1 basepair 3′ of exon 17; c.1845-2_2007+1del; p.Q616_M669del54. DNA sequence is depicted in FIG. 5A and RT-PCR with primers flanking the deletion indicate that the mutant transcript is shorter than the WT transcript (depicted in FIG. 5B). When bred to homozygosity, this mouse is viable and fertile. This is in contrast to almost all other mouse models that indicate that Nf1 nullizygosity is embryonic lethal by E9.5. This provides proof of concept that exon 17 is not essential for neurofibromin function or that at least its loss results in a partially functional protein. There is no obvious phenotype at 4 months of age. The nullizygous mouse appears grossly similar to a wild type matched control (FIG. 5C).

### Example 4—Characterization of Exon Skipping Efficiency of 25-Mer Antisense Oligonucleotides

As discussed above, each of the exons and intronic flanking regions secondary structures were modeled using Visual OMP software in order to assess the biophysical binding properties of the ASOs to target sequences. 25-mer ASOs were designed for exons 17 (SEQ ID NOS: 1-6; target sequences are SEQ ID NOS: 25-30; see FIG. 6C), 47, (SEQ ID NOS: 7-12 target sequences are SEQ ID NOS: 31-36; see FIG. 7C), and 52 (SEQ ID NOS 13-18; target sequences are SEQ ID NOS: 37-42; see FIG. 8C) and tested in cell culture at 2 μM in HEK293T cells. A nested RT-PCR read-out was developed and optimized for detection of the exons skipping in these cell lines. Semi-quantitative analysis of the RT-PCR results was performed using ImageJ software.

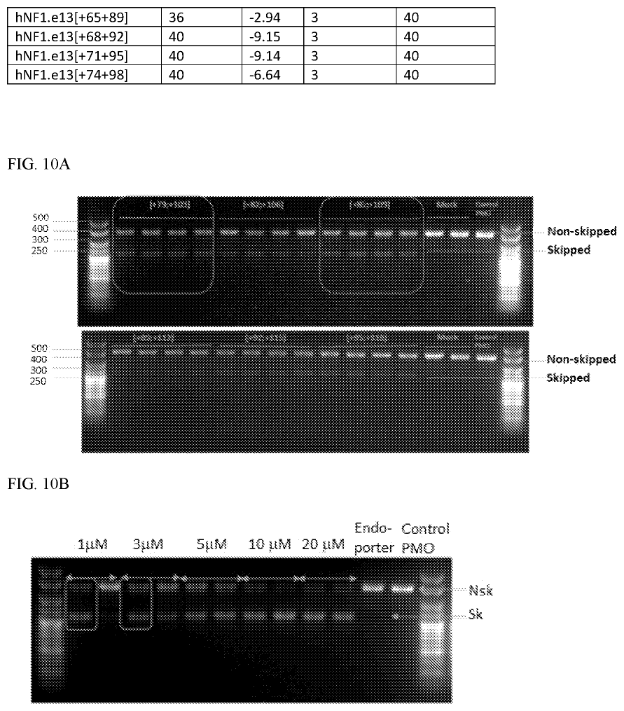

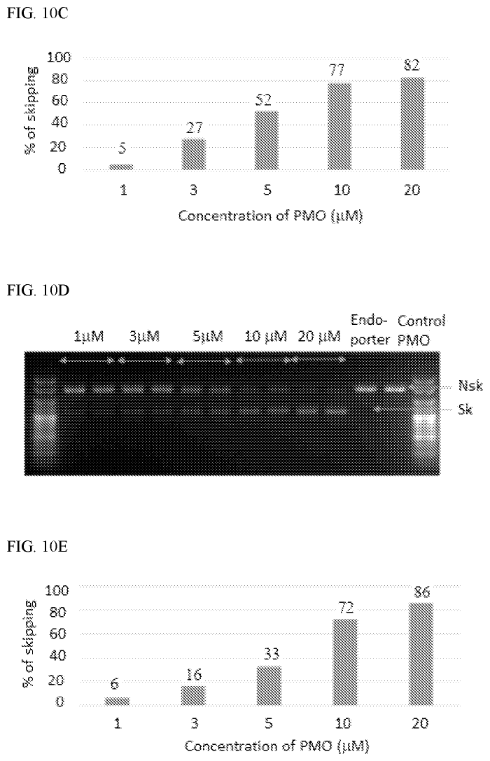

Exemplary results are shown in FIG. 10A for 25-mer ASOs designed for exon skipping of exon 17 in NF1 pre-mRNA. FIG. 10A shows the results of screening 25-mer ASOs hNF1.e17[+79+103] (SEQ ID NO: 1), hNF1.e17[+82+106] (SEQ ID NO: 2), hNF1.e17[+85+109] (SEQ ID NO: 3), hNF1.e17[+89+112] (SEQ ID NO: 4), hNF1.e17[+92+115] (SEQ ID NO: 5), and hNF1.e17[+95+118] (SEQ ID NO: 6) in HEK293 cells expressing wild-type NF1 using the nested PCR readout described in the Methods section. 25-mer ASOs hNF1.e17[+79+103] and hNF1.e17[+85+109] induced the greatest effect of exon skipping. These 25-mer ASOs were further characterized.

As the concentration of ASO hNF1.e17[+79+103] was increased from 1 μM to 20 μM, increased exon skipping was observed in HEK293 cells expressing wild-type NF1 (FIG. 10B). The results shown in FIG. 10B were quantified and shown in FIG. 10C. As shown, the exon skipping efficiency of ASO hNF1.e17[+79+103] in HEK293 cells expressing wild-type NF1 increased from 5% to 82% as the concentration of ASO hNF1.e17[+79+103] was increased from 1 μM to 20 μM.

Likewise, as the concentration of ASO hNF1.e17[+85+109] was increased from 1 μM to 20 μM, increased exon skipping was observed in HEK293 cells expressing wild-type NF1 (FIG. 10D). The results shown in FIG. 10D were quantified and shown in FIG. 10E. The exon skipping efficiency of ASO hNF1.e17[+85+109] in HEK293 cells expressing wild-type NF1 increased from 6% to 86% as the concentration of ASO hNF1.e17[+85+109] was increased from 1 μM to 20 μM.

Similar results were obtained for exons 47 and 52 (data not shown)

These results show the 25-mer ASOs designed as described herein are capable of inducing exon skipping in NF1 pre-mRNA.

### Example 5—Characterization of Exon Skipping Efficiency of 28-Mer Antisense Oligonucleotides

Following dose response examinations of the 25-mer ASOs described in Example 4 above, the most efficacious ASOs were then further optimized by increasing their size to 28-mers and preforming micro walk across their respective target sequences. The 28-mer PMOs show significantly increased skipping compared to their 25-mer counterparts. Representative results for exon skipping induced by 28-mer ASOs in HEK293 cells expressing wild-type NF1 is shown in FIG. 11.

As the concentration of 28-mer ASO hNF1.e17[+79+106] (SEQ ID NO: 49; target sequence is SEQ ID NO: 50; see FIG. 6C) was increased from 500 nM to 10 μM, increased exon skipping was observed in HEK293 cells expressing wild-type NF1 (FIG. 11A). The IC₅₀ of hNF1.e17[+79+106] was determined to be 4 μM.

Results for 28-mer ASOs targeting exons 47 and 52 were also obtained. As the concentration of 28-mer ASO hNF1.e47[+76+103] (SEQ ID NO: 51; target sequence is SEQ ID NO: 52; see FIG. 7C) was increased from 10 nM to 10 μM, increased exon skipping was observed in HEK293 cells expressing wild-type NF1 (FIG. 11B). The IC₅₀ of hNF1.e47[+76+103] was determined to be 300 nM.

These results show the 25-mer ASOs designed as described herein are capable of inducing exon skipping in NF1 pre-mRNA.

### Example 6—In Vitro Models of Exons Skipping

HEK293T cells lines were developed containing a mutation in exons 17, 47, and 52. The cell lines created showed virtually no expression of neurofibromin and increased activation of the Ras pathway.

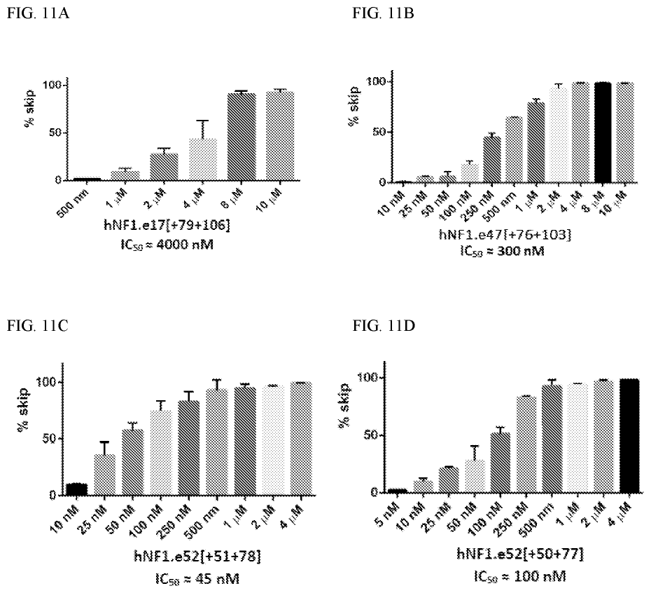

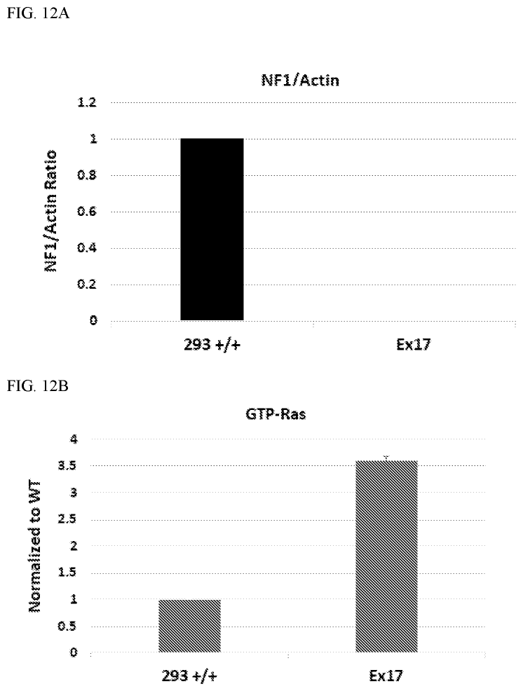

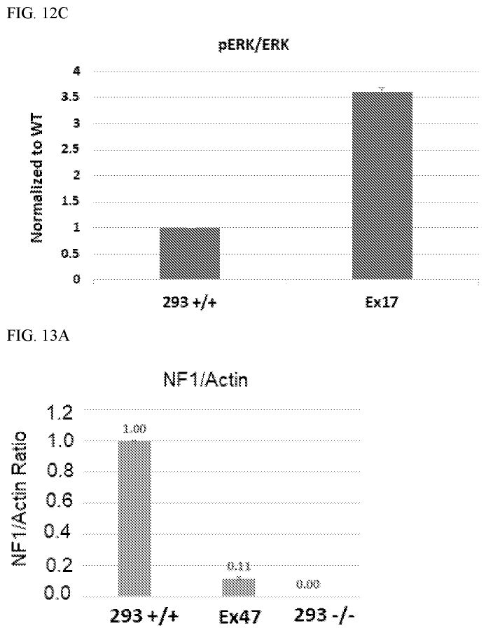

The exon 17 mutations is a patient specific mutation (c.1885G>A; p.G629R; which creates a cryptic splice that results in Gln616Glyfs*4). The cell line containing the mutation (designated Ex17) shows no virtually no expression of neurofibromin as compared to the HEK293T cell line containing with wild-type NF1 (293+/+) as shown in FIG. 12A. As expected, in the absence of neurofibromin polypeptide, the amount of GTP-Ras (FIG. 12B) and the pERK/ERK ratio (FIG. 12C) was significantly increased in the cell line containing the mutation (designated Ex17) as compared to HEK293T 293+/+ cells.

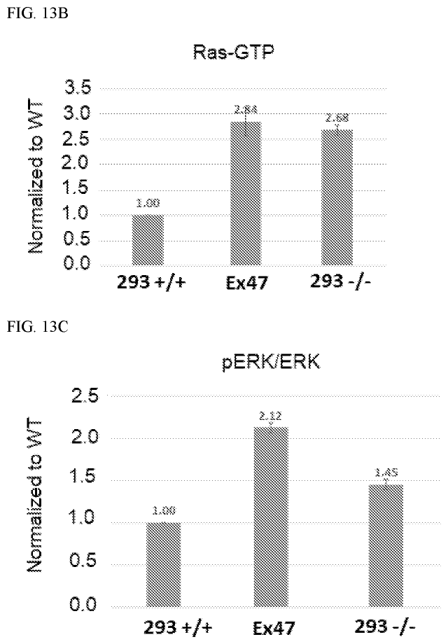

The exon 47 mutation (a homozygous c.6948insT) generates a frameshift mutation. The cell line containing the mutation (designated Ex47) shows significantly reduced expression of neurofibromin as compared to the HEK293T cell line containing wild-type NF1 (293+/+) and HEK293T cells that do not express NF1 (293^{-/-}) as shown in FIG. 13A. As expected, in the presence of reduced neurofibromin polypeptide, the amount of GTP-Ras (FIG. 13B) and the pERK/ERK ratio (FIG. 13C) was significantly increased in the cell line containing the mutation (designated Ex47) as compared to HEK293T 293+/+ and HEK293−/− cells.

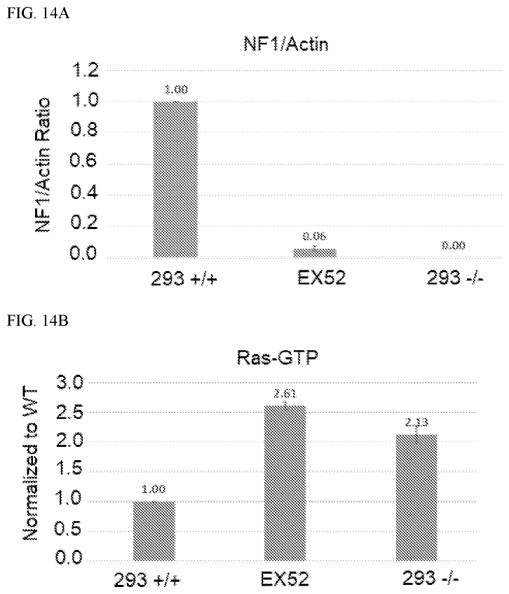

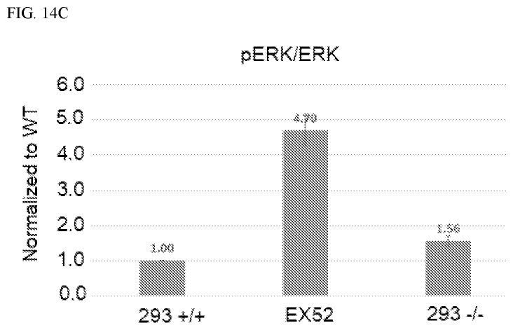

The exon 52 mutation is also contains a patient specific mutation (c.7648A>T; p.R2550X). This cell line is not homozygous for the mutation and contains at least two additional mutations. As with the cell lines expressing mutations in exons 17 and 47, HEK293T cells expressing this mutation shows significantly reduced expression of neurofibromin (FIG. 14A), increased amounts of GTP-Ras (FIG. 14B), and an increased pERK/ERK ratio (FIG. 14C).

The cells lines generated above are conveniently used to evaluate exon skipping of the ASOs described herein.

### Example 7—Exon Skipping of Exons 17 and 52 Using Antisense Oligonucleotides

The cell lines described in Example 7 were used to evaluate the effects of exon skipping of exons 17 and 52. In this example, 10 μM of ASOs specific for exon 17 ( ) and exon 52 ( ) were incubated with HEK293 cells for 48 hours. After incubation, the effect of the ASOs on neurofibromin expression and pERK/ERK ratios were examined.

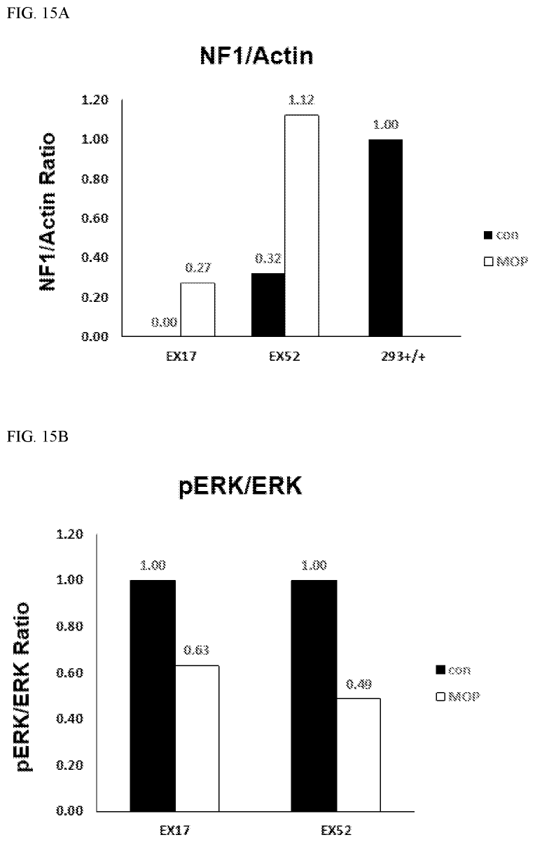

The results for neurofibromin expression are shown in FIG. 15A. Western blots of mutant cells treated with control ASOs (con) and exon 17 and 52 specific ASOs (MOP) were quantitated along with neurofibromin expression of HEK293 cells expressing wild-type NF1 (293+/+) and HEK293 NF1 null cells (293−/−). The results show that exon 17 and exon 52 specific ASOs were able to induce exon skipping and at least partially restore neurofibromin polypeptide expression. As seen in Example 4 above, exon skipping of exon 52 resulted in increased levels of neurofibromin expression (compare exon 52 MOP to 293+/+).

The results for pERK/ERK ratio are shown in FIG. 15B. Western blots of mutant cells treated with control ASOs (con) and exon 17 and 52 specific ASOs (MOP) were quantitated. As expected, in the presence of functional neurofibromin expression induced by exon skipping of exons 17 and 52, the pERK/ERK ration was decreased in exon 17 and 52 HEK293 cells as compared to control HEK293 cells.

This example shows exon skipping of exons 17 and 52 is able to restore NF1 protein expression to variable levels in the respective exon 17 and 52 mutant cell lines and also decrease the pERK/ERK ratio, indicating down regulation of the Ras signaling pathway in the mutant cell lines.

### Materials and Methods

### In Silico Analysis

Human NF1 cDNA sequence was downloaded and coding frames and exon boundaries were mapped to identify which exons could be skipped either as singletons or as two consecutive exons while still maintaining protein reading frame. Known and perspective protein domains were also overlaid onto this map. Exon lengths were determined. The literature, the Human Gene Mutation Database (HGMD), and the Leiden Open Variation Database was reviewed for reports of patients with known NF1 mutations that result in skipping of exons during splicing. In addition, experimentally verified posttranslational modifications (PTM) of NF1 as reported on UniProtKB (https://www.uniprot.org/uniprot/P21359), PhosphoNet (http://www.phosphonet.ca), and Phosphosite Plus (http://www.phosphosite.org4) were mapped. PTMs included phosphorylation, acetylation, methylation, and ubiquitination. Further, neurofibromin (P21359-2) was analyzed using PredictProtein (https://www.predictprotein.org) (Yachdav, et al., (2014), Nucleic Acids Res 42, W337-343) a web server that combines various protein sequence analysis and structure prediction tools. Specifically, PredictProtein returns predictions of protein secondary structure, solvent accessibility, disorder status, protein-protein binding, PTMs, and conservation, among others. Conservation scores were obtained via ProteinPredict using ConSurf (https://consurf.tau.ac.il) (Ashkenazy, et al., (2016), Nucleic Acids Res 44, W344-350). For PTM predictions based on signature sequences, ProteinPredict calls Prosite (https://prosite.expasy.org) (Sigrist, et al., (2002), Brief Bioinform 3, 265-274). Outputs were analyzed for all 58 exons, individually. Note that unlike other structure prediction tools, inputs to PredictProtein are not limited by their sequence length, which makes this tool particularly useful given neurofibromin's length.

For a selection of 12 exons, 11 single and one pair of consecutive exons, PredictProtein was also run on the amino acid sequences obtained by skipping the exon(s) Resulting predictions of various features were compared to those previously obtained for neurofibromin (see above) in order to assess the impact that individual exon deletions have on the protein structure and functionality. Residue-specific differences were quantified and summarized. In addition, we predicted ubiquitination sites in selected exons using the tools CKSAAP_UbSite (http://systbio.cau.edu.cn/cksaau_ubsite/) (Chen, et al., (2013), Biochim Biophys Acta 1834, 1461-1467) and UbiProber (http://bioinfo.ncu.edu.cn/UbiProber.aspx) (Chen, et al., (2013), Bioinformatics 29, 1614-1622).

cDNA Expression System

A heterologous cell culture expression system has been established using a full-length mNf1 and NF1-null human cell lines (Wallis, et al., (2018), Hum Mutat 39, 816-821). The full-length mNf1 cDNA produces a >250 kDa neurofibromin protein that is capable of modulating Ras signaling. Mutant cDNAs representing various exon skips were created and their ability to produce mature neurofibromin and restore Nf1 activity in NF1^{-/-} cells was assessed. Ras activity data for cDNAs included levels of GTP-Ras, p-ERK/ERK ratios, and ELK1 transcriptional activity normalized to wild type (WT) for each experiment and in comparison to empty vector (EV) cDNA.

### Cell Culture

HEK293 (WT or NF1+/+) cells were obtained from ATCC (CRL-1573) and cultured in DMEM+10% FBS and 1× Pen/Strep using standard culture procedures. NF1 −/− or null HEK293 cells were created through CRISPR Cas9 targeting NF1 exon 2 (Wallis, et al., (2018), Hum Mutat 39, 816-821).

Nf1 cDNA Plasmid Development

The Nf1 cDNA plasmid was developed by GeneCopoeia and is commercially available.

### Transient Transections

HEK293 WT or null cells were transfected with LipoD293 (SignaGen Lab. Cat #SL 100668) or Lipofectamie 3000 (Invitrogen cat #L30000008) and cDNA at 1 ug per 6-well dish seeded with 500,000 cells per well or 100 ng/96-well seeded with 50,000 cells. Assays were performed 48-72 hours later.

### Western Blotting

Cells were lysed with RIPA buffer and lysates were cleared by centrifugation at 20,000 RPM for 20 minutes at 4 C. Protein was quantitated with a Bradford assays and 50 ug of protein was loaded per well for NF1 blots and 10 ug of protein was loaded for other blots. 8% SDS-polyacrylamide gels were run at 100 V for 2 hours and transferred at 100 V for 2 hours onto PVDF. Blots were probed overnight at 4 C with primary antibody washed and probed 1 hour at room temperature with secondary. Primary antibodies include N-Terminal NF1 (Cell Signaling cat #D7R7D 1:1000), tubulin (Abcam cat #ab52866 1:1000), p-ERK (Cell Signaling cat #9101 1:1000), and total ERK (Cell Signaling cat #9102 1:1000). Secondary was HRP tagged from Santa Cruz. Chemiluminescent substrate from Bio-Rad was used as per manufacturer's protocols.

### RAS-G-LISA Assay

The RAS-G-LISA assay was obtained from Cytoskelton Inc. (catalog no. BK131) and was performed according to the manufacturer's instructions. Briefly, The RAS-G-LISA kit is contains a Ras GTP-binding protein linked to the wells of a 96 well plate. Active, GTP-bound Ras in a cell/tissue lysate will bind to the wells while inactive GDP-bound Ras is removed during washing steps. The bound active Ras is detected with a Ras specific antibody. The degree of Ras activation is determined by comparing readings from activated cell lysates versus non-activated cell lysates. Inactivation of Ras is generally achieved in tissue culture by a serum starvation step. For Ras activation assays, adherent cells are grown to 50-70% confluency, for example over a period of 3-5 days under normal culture conditions appropriate for the cell. Cells are serum starved (for example, from 12-24 hours) and then stimulated with an appropriate ligand for Ras activation (for example, epidermal growth factor). The cells are lysed with a lysis buffer provided containing protease inhibitors and optional phosphatase inhibitors and standardized by protein concentration and added to the 96 well plate. After washing and antibody incubation steps, detection of bound GTP-bound Ras is determined by measuring absorbance (for example at 490 nm) using a microplate spectrophotometer.

### ELK-1 Transcriptional Repression Assay

ELK1 is a major nuclear substrate for ERK, where phosphorylation of ELK1 by kinases results in the conformational change of ELK1 and triggers its DNA binding activity. Plasmids containing the ELK-1 transactivation domain fused to GAL-4 and UAS-Luciferase constructs were a kind gift from the Roger Davis laboratory. Together, they act as a reporter system to monitor ELK1-dependent transcriptional activity and MAPK signaling. In fact, ERK suppression has been measured by the ELK reporter assay in HEK293 cells to show that SPRED1 recruits NF1 to suppress Ras activation. Both NF1 and SPRED1 mutations in the GRD-EVH1 interaction domains reduce Ras-ERK suppression activity¹⁷. A strong correlation among pathogenic mutations, disruption of the GRD-EVH1 interaction, and ERK suppression activity has been reported¹⁷. Hence, NF1 −/− HEK 293 cells were transfected with 25 ng of pGAL4 and pGal4-ELK1 plasmids, 1 ng pNL1.1TK [Nluc/TK] transfection control, along with 100 ng of respective Nf1 mutation plasmids with Lipofectamine 3000 and plated in a 96 well plate such that each well received 50,000 cells. After 24 hours, the medium was replaced with normal growth medium. The experiment was terminated at 48 hours after transfection with reporter lysis buffer. After lysis nanoluc and firefly luciferase readings (Relative Light Units, RLUs) were obtained using Luciferase Assay Reagent (Promega, E1500) and a BioTek Synergy 2 plate reader. Readings were normalized to NanoLuc expression and percentage change in luciferase activity in comparison to NF1 −/− cells transfected with WT cDNA vector. We evaluated statistical deviation of each cDNA from the empty vector cDNA clone to evaluate if cDNAs retained any function. Each mutation set was done in triplicate and the entire experimental set up was repeated at least three times.

### Nested PCR Readout

RNA was harvested and DNase-treated using Qiagen RNeasy kit. 600 ng of RNA used to produce cDNA using Promega GoScript RT system (in 20 μl). 1 μl of cDNA was then used in 25 μl PCR reaction using 1^{st} round primers with 20 cycles of amplification at an appropriate annealing temperature, then 2^{nd} round primers used to amplify 1 μl of 1^{st} round product with 30 cycles at an appropriate annealing temperature. PCR was performed using Platinum HotStart PCR master mix or GoTaq polymerase. 10 μl of product was then run on an appropriate % agarose gel and the percent exon skipping quantified using Image J densitometric analysis. Primers for the nested PCR reaction for exons 17 (SEQ ID NOS: 72-75), 47 (SEQ ID NOS: 76-79) and 52 (SEQ ID NOS: 80-83) are shown in Table 2 below.

TABLE 2

| | | | | | Size | |
|---|---|---|---|---|---|---|
| | | | | Size | Not | |
| | PCR | Primer | Primer Sequence | Skipped | Skipped | TM |
| Exon | Round | Direction | (5′-3′) | (bp) | (bp) | (° C.) |
| 17 | 1 | Forward | AATGGAGGCTCTGCTGGTTC | 389 | 545 | 60.03 |
| | 1 | Reverse | ACACTTCATCCACCCCACAC | | | 59.89 |
| | 2 | Forward | TCTCAAGTGGTTGCGGGAAA | 209 | 365 | 59.82 |
| | 2 | Reverse | CTGCTTCCTCACAGAGGTGG | | | 60.04 |
| 47 | 1 | Forward | TCCATCCCTGCAACCAAGAG | 348 | 489 | 59.67 |
| | 1 | Reverse | GCAGGTGAAGGATGCCTGTA | | | 59.75 |
| | 2 | Forward | CGAGTGTCTCATGGGCAGAT | 213 | 354 | 59.54 |
| | 2 | Reverse | ATCCATTTGCTTGCAGTGCC | | | 59.75 |
| 52 | 1 | Forward | AAGGATACCTTGCAGCCACC | 323 | 446 | 60.03 |
| | 1 | Reverse | CACAACACTGGCCTCTGCTA | | | 59.96 |
| | 2 | Forward | GCCAGGAAATCCATGAGCCT | 98 | 221 | 60.11 |
| | 2 | Reverse | GGGTGCTGTTGTGATGAGGA | | | 59.96 |

### Mutant Mouse Generation

Exon 17 was deleted from mouse using CRISPR Cas9 technology. Guides were made to regions immediately flanking exon 17 in the mouse genome: 5′ guide: GGATACGTCTTCTITCCAGC (SEQ ID NO: 70) and 3′ guide: TGGTAGGTAACTCCCTCTGT (SEQ ID NO: 71). This resulted in an allele with all of exon 17 deleted including the canonical ΔG splice site at the 3′ end of intron 16 flanking exon 17 as well as the +1 site of intron 17; c.1845-2_2007+1del; p.Q616_M669del54. This mouse was bred to homozygosity.

1. An isolated antisense oligonucleotide that specifically hybridizes to a target sequence comprising a continuous stretch of at least 20 nucleotides within NF1 pre-mRNA from at least one of exons 17, 52, 47, 9, 12, 13, 20, 21, 25, 36, or 41.

2. The isolated antisense oligonucleotide of claim 1, wherein the antisense oligonucleotide contains from 20 to 30 nucleotides or bases.

3. The isolated antisense oligonucleotide of claim 1, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 49 and specifically hybridizes to a continuous stretch of at least 20 nucleotides within SEQ ID NO: 57.

4. (canceled)

5. (canceled)

6. (canceled)

7. The isolated antisense oligonucleotide of claim 1, wherein the antisense oligonucleotide is selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 51 and specifically hybridizes to a continuous stretch of at least 20 nucleotides within SEQ ID NO: 63.

8. (canceled)

9. (canceled)

10. (canceled)

11. The isolated antisense oligonucleotide of claim 1, wherein the antisense oligonucleotide is selected from the group consisting of SEQ ID NO: 13, SEQ ID NO: 14, SEQ ID NO: 15, SEQ ID NO: 16, SEQ ID NO: 17, SEQ ID NO: 18, or SEQ ID NO: 53 and specifically hybridizes to a continuous stretch of at least 20 nucleotides within SEQ ID NO: 64.

12. (canceled)

13. (canceled)

14. (canceled)

15. The isolated antisense oligonucleotide of claim 1, wherein the antisense oligonucleotide is selected from the group consisting of SEQ ID NO: 19, SEQ ID NO: 20, SEQ ID NO: 21, SEQ ID NO: 22, SEQ ID NO: 23, or SEQ ID NO: 24 and specifically hybridizes to a continuous stretch of at least 20 nucleotides within SEQ ID NO: 65.

16. (canceled)

17. The isolated antisense oligonucleotide of claim 1, wherein the antisense oligonucleotide contains at least one residue that is modified to increase nuclease resistance, to increase the affinity of the oligonucleotide for the target nucleotide sequence, or a combination of the foregoing.

18. The isolated antisense oligonucleotide of claim 1, wherein the antisense oligonucleotide comprises a non-natural backbone.

19. (canceled)

20. The isolated antisense oligonucleotide of claim 1, wherein the antisense oligonucleotide is a phosphorodiamidate morpholino oligonucleotide.

21. A composition comprising an isolated antisense oligonucleotide that specifically hybridizes to a continuous stretch of at least 20 nucleotides within NF1 pre-mRNA from at least one of exons 17, 52, 47, 9, 12, 13, 20, 21, 25, 36, or 41.

22. The composition of claim 21, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 49 and specifically hybridizes to a continuous stretch of at least 20 nucleotides within SEQ ID NO: 57.

23. (canceled)

24. (canceled)

25. (canceled)

26. The composition of claim 21, wherein the antisense oligonucleotide is selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 51 and specifically hybridizes to a continuous stretch of at least 20 nucleotides within SEQ ID NO: 63.

27. (canceled)

28. (canceled)

29. (canceled)

30. The composition of claim 21, wherein the antisense oligonucleotide is selected from the group consisting of SEQ ID NO: 13, SEQ ID NO: 14, SEQ ID NO: 15, SEQ ID NO: 16, SEQ ID NO: 17, SEQ ID NO: 18, or SEQ ID NO: 53 and specifically hybridizes to a continuous stretch of at least 20 nucleotides within SEQ ID NO: 64.

31. (canceled)

32. (canceled)

33. (canceled)

34. The composition of claim 21, wherein the antisense oligonucleotide is selected from the group consisting of SEQ ID NO: 19, SEQ ID NO: 20, SEQ ID NO: 21, SEQ ID NO: 22, SEQ ID NO: 23, or SEQ ID NO: 24 and specifically hybridizes to a continuous stretch of at least 20 nucleotides within SEQ ID NO: 65.

35. (canceled)

36. (canceled)

37. (canceled)

38. (canceled)

39. (canceled)

40. A method for treating a subject suffering from a disease or condition associated with a mutation in a NF1 gene encoding a neurofibromin polypeptide, the method comprising administering to a subject a therapeutically effective amount of an antisense oligonucleotide comprising a sequence that is specifically hybridisable to a target sequence in a NF1 pre-mRNA exon from at least one of exons 9, 12, 13, 17, 20, 21, 25, 36, 41, 47, and 52.

41. The method of claim 40, wherein the exon is exon 17, the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 49, the target sequence is SEQ ID NO: 57 and the administering step results in at least partial skipping of exon 17.

42. (canceled)

43. (canceled)

44. (canceled)

45. The method of claim 40, wherein the exon is exon 47, the antisense oligonucleotide is selected from the group consisting of SEQ ID NO: 7, SEQ ID NO: 8, SEQ ID NO: 9, SEQ ID NO: 10, SEQ ID NO: 11, SEQ ID NO: 12, SEQ ID NO: 51, the target sequence is SEQ ID NO: 63 and the administering step results in at least partial skipping of exon 47.

46. (canceled)

47. (canceled)

48. (canceled)

49. The method of claim 40, wherein the exon is exon 52, the antisense oligonucleotide is selected from the group consisting of SEQ ID NO: 13, SEQ ID NO: 14, SEQ ID NO: 15, SEQ ID NO: 16, SEQ ID NO: 17, SEQ ID NO: 18, or SEQ ID NO: 53, the target sequence is SEQ ID NO: 64 and the administering step results in at least partial skipping of exon 52.

50. (canceled)

51. (canceled)

52. (canceled)

53. The method of claim 40, wherein the exon is exon 13, the antisense oligonucleotide is selected from the group consisting of SEQ ID NO: 19, SEQ ID NO: 20, SEQ ID NO: 21, SEQ ID NO: 22, SEQ ID NO: 23, or SEQ ID NO: 24, the target sequence is SEQ ID NO: 65 and the administering step results in at least partial retention of exon 13.

54. (canceled)

55. The method of claim 40, wherein the antisense oligonucleotide contains at least one residue that is modified to increase nuclease resistance, to increase the affinity of the oligonucleotide for the target nucleotide sequence, or a combination of the foregoing.

56. (canceled)

57. (canceled)

58. (canceled)


SEQUENCE LISTING (83 sequences)

SEQ ID NO: 1 - DNA, 25 bp, Artificial Sequence
  ASO for NF1 exon 17
attcttcatgatccatggacatttg

SEQ ID NO: 2 - DNA, 25 bp, Artificial Sequence
  ASO for NF1 exon 17
gtaattcttcatgatccatggacat

SEQ ID NO: 3 - DNA, 25 bp, Artificial Sequence
  ASO for exon 17
gtagtaattcttcatgatccatgga

SEQ ID NO: 4 - DNA, 25 bp, Artificial Sequence
  ASO for exon 17
tacgtagtaattcttcatgatccat

SEQ ID NO: 5 - DNA, 25 bp, Artificial Sequence
  ASO for NF1 exon 17
caggagtacgtagtaattcttcatg

SEQ ID NO: 6 - DNA, 25 bp, Artificial Sequence
  ASO for exon 17
ctccaggagtacgtagtaattcttc

SEQ ID NO: 7 - DNA, 25 bp, Artificial Sequence
  ASO for NF1 exon 47
tcatcaagctgcagcacagccacag

SEQ ID NO: 8 - DNA, 25 bp, Artificial Sequence
  ASO for NF1 exon 47
acctcatcaagctgcagcacagcca

SEQ ID NO: 9 - DNA, 25 bp, Artificial Sequence
  ASO for NF1 exon 47
ttgacctcatcaagctgcagcacag

SEQ ID NO: 10 - DNA, 25 bp, Artificial Sequence
  ASO for NF1 exon 47
tttgttcaagaagtgcggtacctgc

SEQ ID NO: 11 - DNA, 25 bp, Artificial Sequence
  ASO for NF1 exon 47
ggttttgttcaagaagtgcggtacc

SEQ ID NO: 12 - DNA, 25 bp, Artificial Sequence
  ASO for NF1 exon 47
gcaggttttgttcaagaagtgcggt

SEQ ID NO: 13 - DNA, 25 bp, Artificial Sequence
  ASO for NF1 exon 52
tctgatatcaagtgatcaaaacttt

SEQ ID NO: 14 - DNA, 25 bp, Artificial Sequence
  ASO for NF1 exon 52
tgatccctgattccatttcttgcct

SEQ ID NO: 15 - DNA, 25 bp, Artificial Sequence
  ASO for NF1 exon 52
ctactctcctcattttggggggtgt

SEQ ID NO: 16 - DNA, 25 bp, Artificial Sequence
  ASO for NF1 exon 52
ctgctactctcctcattttgggggg

SEQ ID NO: 17 - DNA, 25 bp, Artificial Sequence
  ASO for NF1 exon 52
tttctgctactctcctcattttggg

SEQ ID NO: 18 - DNA, 25 bp, Artificial Sequence
  ASO for NF1 exon 52
cagtttctgctactctcctcatttt

SEQ ID NO: 19 - DNA, 25 bp, Artificial Sequence
  ASO for NF1 exon 13
tacttacagcttcttgtctccaggt

SEQ ID NO: 20 - DNA, 25 bp, Artificial Sequence
  ASO for NF1 exon 13
agaagatacttacagcttcttgtct

SEQ ID NO: 21 - DNA, 25 bp, Artificial Sequence
  ASO for NF1 exon 13
aagagaagatacttacagcttcttg

SEQ ID NO: 22 - DNA, 25 bp, Artificial Sequence
  ASO for NF1 exon 13
gacaagagaagatacttacagcttc

SEQ ID NO: 23 - DNA, 25 bp, Artificial Sequence
  ASO for NF1 exon 13
atggacaagagaagatacttacagc

SEQ ID NO: 24 - DNA, 25 bp, Artificial Sequence
  ASO for NF1 exon 13
accatggacaagagaagatacttac

SEQ ID NO: 25 - DNA, 25 bp, Homo sapiens
caaatgtccatggatcatgaagaat

SEQ ID NO: 26 - DNA, 25 bp, Homo sapiens
atgtccatggatcatgaagaattac

SEQ ID NO: 27 - DNA, 25 bp, Homo sapiens
tccatggatcatgaagaattactac

SEQ ID NO: 28 - DNA, 25 bp, Homo sapiens
atggatcatgaagaattactacgta

SEQ ID NO: 29 - DNA, 25 bp, Homo sapiens
catgaagaattactacgtactcctg

SEQ ID NO: 30 - DNA, 25 bp, Homo sapiens
gaagaattactacgtactcctggag

SEQ ID NO: 31 - DNA, 25 bp, Homo sapiens
ctgtggctgtgctgcagcttgatga

SEQ ID NO: 32 - DNA, 25 bp, Homo sapiens
tggctgtgctgcagcttgatgaggt

SEQ ID NO: 33 - DNA, 25 bp, Homo sapiens
ctgtgctgcagcttgatgaggtcaa

SEQ ID NO: 34 - DNA, 25 bp, Homo sapiens
gcaggtaccgcacttcttgaacaaa

SEQ ID NO: 35 - DNA, 25 bp, Homo sapiens
ggtaccgcacttcttgaacaaaacc

SEQ ID NO: 36 - DNA, 25 bp, Homo sapiens
accgcacttcttgaacaaaacctgc

SEQ ID NO: 37 - DNA, 25 bp, Homo sapiens
aaagttttgatcacttgatatcaga

SEQ ID NO: 38 - DNA, 25 bp, Homo sapiens
aggcaagaaatggaatcagggatca

SEQ ID NO: 39 - DNA, 25 bp, Homo sapiens
acaccccccaaaatgaggagagtag

SEQ ID NO: 40 - DNA, 25 bp, Homo sapiens
ccccccaaaatgaggagagtagcag

SEQ ID NO: 41 - DNA, 25 bp, Homo sapiens
cccaaaatgaggagagtagcagaaa

SEQ ID NO: 42 - DNA, 25 bp, Homo sapiens
aaaatgaggagagtagcagaaactg

SEQ ID NO: 43 - DNA, 25 bp, Homo sapiens
acctggagacaagaagctgtaagta

SEQ ID NO: 44 - DNA, 25 bp, Homo sapiens
agacaagaagctgtaagtatcttct

SEQ ID NO: 45 - DNA, 25 bp, Homo sapiens
caagaagctgtaagtatcttctctt

SEQ ID NO: 46 - DNA, 25 bp, Homo sapiens
gaagctgtaagtatcttctcttgtc

SEQ ID NO: 47 - DNA, 25 bp, Homo sapiens
gctgtaagtatcttctcttgtccat

SEQ ID NO: 48 - DNA, 25 bp, Homo sapiens
gtaagtatcttctcttgtccatggt

SEQ ID NO: 49 - DNA, 28 bp, Artificial Sequence
  ASO for NF1 exon 17
gtaaatcttcatgatccatggacatttg

SEQ ID NO: 50 - DNA, 28 bp, Homo sapiens
caaatgtccatggatcatgaagaattac

SEQ ID NO: 51 - DNA, 28 bp, Artificial Sequence
  ASO for NF1 exon 47
aggttttgttcaagaagtgcggtacctg

SEQ ID NO: 52 - DNA, 28 bp, Homo sapiens
caggtaccgcacttcttgaacaaaacct

SEQ ID NO: 53 - DNA, 28 bp, Artificial Sequence
  ASO for NF1 exon 52
ttgtgatccctgattccatttcttgcct

SEQ ID NO: 54 - DNA, 28 bp, Homo sapiens
aggcaagaaatggaatcagggatcacaa

SEQ ID NO: 55 - DNA, 174 bp, Homo sapiens
aagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaag

SEQ ID NO: 56 - DNA, 132 bp, Homo sapiens
tccgcattggattggtggcctaagattgatgctgtgtattgtcactcggttgaacttcgaaatatgtttggtgaaacacttcataaagcagtgcaaggttgtggagcacacccagcaatacgaatggcaccg

SEQ ID NO: 57 - DNA, 156 bp, Homo sapiens
caggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatg

SEQ ID NO: 58 - DNA, 84 bp, Homo sapiens
gcttgggaagatacacatgcaaaatgggaacaagcaacaaagctaatccttaactatccaaaagccaaaatggaagatggccag

SEQ ID NO: 59 - DNA, 441 bp, Homo sapiens
gctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacag

SEQ ID NO: 60 - DNA, 117 bp, Homo sapiens
agatttggaccaggcaagcatggaagcagtagtttcacttctagctggtctccctctgcagcctgaagaaggagatggtgtggaattgatggaagccaaatcacagttatttcttaa

SEQ ID NO: 61 - DNA, 111 bp, Homo sapiens
gcatcaggtacatgaaaaagaagaattcaaggctttgaaaacgttaagtattttctaccaagctgggacttccaaagctgggaatcctattttttattatgttgcacggag

SEQ ID NO: 62 - DNA, 141 bp, Homo sapiens
attacagatctgcttgatgttgtactagacagtttcatcaaaaccagtgcaacaggtggcttgggatcaataaaagctgaggtgatggcagatactgctgtagctttggcttctggaaatgtgaaattggtttcaagcaag

SEQ ID NO: 63 - DNA, 141 bp, Homo sapiens
gactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaag

SEQ ID NO: 64 - DNA, 123 bp, Homo sapiens
gaacaaggaaaagttttgatcacttgatatcagacacaaaggctcctaaaaggcaagaaatggaatcagggatcacaacaccccccaaaatgaggagagtagcagaaactgattatgaaatgg

SEQ ID NO: 65 - DNA, 135 bp, Homo sapiens
agtcttacatttaaagaaaaagtaacaagccttaaatttaaagaaaaacctacagacctggagacaagaagctataagtatcttctcttgtccatggtgaaactaattcatgcagatccaaagctcttgctttgt

SEQ ID NO: 66 - DNA, 50 bp, Mus musculus
tcaggaggatacgtcttctttccagcaggcagatagaagctcctgtcatt

SEQ ID NO: 67 - DNA, 50 bp, Mus musculus
gaaaggaagagggaattcctccatggtaggtaactccctctgtgactctc

SEQ ID NO: 68 - DNA, 23 bp, Mus musculus
tcaggaggatacgtcttctttcc

SEQ ID NO: 69 - DNA, 24 bp, Mus musculus
taggtaactccctctgtgactctc

SEQ ID NO: 70 - DNA, 20 bp, Artificial Sequence
  CRISPR guide
ggatacgtcttctttccagc

SEQ ID NO: 71 - DNA, 20 bp, Artificial Sequence
  CRISPR guide
tggtaggtaactccctctgt

SEQ ID NO: 72 - DNA, 20 bp, Artificial Sequence
  PCR primer
aatggaggctctgctggttc

SEQ ID NO: 73 - DNA, 20 bp, Artificial Sequence
  PCR primer
acacttcatccaccccacac

SEQ ID NO: 74 - DNA, 20 bp, Artificial Sequence
  PCR primer
tctcaagtggttgcgggaaa

SEQ ID NO: 75 - DNA, 20 bp, Artificial Sequence
  PCR primer
ctgcttcctcacagaggtgg

SEQ ID NO: 76 - DNA, 20 bp, Artificial Sequence
  PCR primer
tccatccctgcaaccaagag

SEQ ID NO: 77 - DNA, 20 bp, Artificial Sequence
  PCR primer
gcaggtgaaggatgcctgta

SEQ ID NO: 78 - DNA, 20 bp, Artificial Sequence
  PCR primer
cgagtgtctcatgggcagat

SEQ ID NO: 79 - DNA, 20 bp, Artificial Sequence
  PCR primer
atccatttgcttgcagtgcc

SEQ ID NO: 80 - DNA, 20 bp, Artificial Sequence
  PCR primer
aaggataccttgcagccacc

SEQ ID NO: 81 - DNA, 20 bp, Artificial Sequence
  PCR primer
cacaacactggcctctgcta

SEQ ID NO: 82 - DNA, 20 bp, Artificial Sequence
  PCR primer
gccaggaaatccatgagcct

SEQ ID NO: 83 - DNA, 20 bp, Artificial Sequence
  PCR primer
gggtgctgttgtgatgagga